(19) United States
(12) Patent Application Publication
(10) Pub. No.: US 2021/0275686 A1
(43) Pub. Date: Sep. 9, 2021

(54) BINDER/ACTIVE AGENT CONJUGATES DIRECTED AGAINST CXCR5, HAVING ENZYMATICALLY CLEAVABLE LINKERS AND IMPROVED ACTIVITY PROFILE

(71) Applicant: BAYER AKTIENGESELLSCHAFT, Leverkusen (DE)

(72) Inventors: Sarah Anna Liesa JOHANNES, Hilden (DE); Hans-Georg LERCHEN, Leverkusen (DE); Beatrix STELTE-LUDWIG, Wülfrath (DE); Pascale LEJEUNE, Berlin (DE); Hannah JÖRISSEN, Heiligenhaus (DE); Christoph MAHLERT, Wuppertal (DE); Simone GREVEN, Dormagen (DE); Stephan MÄRSCH, Köln (DE); Stefanie HAMMER, Berlin (DE)

(21) Appl. No.: 17/253,086

(22) PCT Filed: Jun. 13, 2019
(86) PCT No.: PCT/EP2019/065517

§ 371 Date: Dec. 16, 2020

(30) Foreign Application Priority Data
Jun. 18, 2018 (EP) 18178299.6

(51) Int. Cl. A61K 47/68; C07K 16/28; A61K 31/40; A61P 35/00

(52) U.S. Cl. CPC A61K 47/6889; A61K 47/6803; A61K 47/6849; C07K 2317/77; A61K 31/40; A61P 35/00; C07K 16/2866

(57) ABSTRACT

The invention relates to a new binder-drug conjugates with improved properties, to active metabolites of said ADCs and to processes for the preparation thereof. The invention particularly relates to antibody-drug conjugates (ADCs) with CXCR5 antibodies and selected KSP inhibitors. The present invention further relates to the use of said conjugates for the treatment and/ore prevention of diseases and to the use of said conjugates for the production of medicaments for the treatment and/or prevention of diseases, in particular hyperproliferative and/or angiogenic diseases such as, for example, cancer diseases.

### INTRODUCTION AND PRIOR ART

The invention relates to novel binder/active agent conjugates, for example antibody-drug-conjugates (ADCs), with improved properties, active metabolites of these binder/active agent conjugates and processes for the preparation thereof. The present invention further relates to the use of these conjugates for the treatment and/or prevention of diseases and the use of said conjugates for the production of medications, particularly of hyperproliferative and/or angiogenic diseases such as cancers. Such treatments can be done as monotherapy or in combination with other medications or additional therapeutic measures. According to the invention, the binder is preferably an antibody.

Cancers are the result of uncontrolled cell growth of a great variety of tissues. In many cases the new cells penetrate into existing tissue (invasive growth), or the metastasize into remote organs. Cancers occur in a great variety of organs and often have tissue-specific disease courses. Therefore, the term “cancer” as a generic term describes a large group of defined diseases of different organs, tissues and cell types.

Some tumors in early stages can be removed by surgical and radiotherapy measured. Metastasized tumors generally only be treated palliatively with chemotherapeutic agents. The goal in such cases is to achieve the optimal combination of improvement of the quality of life and prolonging life.

Conjugates of binder proteins with one or more active agent molecules are known, particularly in the form of so-called “antibody drug conjugates” (ADCs), in which an internalizing antibody directed against a tumor-associated antigen is covalently bonded via a binding unit (“linker”) to a cytotoxic agent. Following introduction of the ADC into the tumor cell and subsequent dissociation of the conjugate, either the cytotoxic agent itself or another cytotoxic metabolite formed therefrom is released within the tumor cell and can exert its effect there directly and selectively. In this way, damage to normal tissue can be kept within significantly narrower limits compared with conventional chemotherapy [see, for example, J. M. Lambert, Curr. Opin. Pharmacol. 5, 543-549 (2005); A. M. Wu and P. D. Senter, Nat. Biotechnol. 23, 1137-1146 (2005); P. D. Senter, Curr. Opin. Chem. Biol. 13, 235-244 (2009); L. Ducry and B. Stump, Bioconjugate Chem. 21, 5-13 (2010)]. WO2012/171020 describes ADCs in which a plurality of toxophore molecules are attached to an antibody via a polymeric linker. Possible toxophores are mentioned in WO2012/171020, including the substances SB 743921, SB 715992 (ispinesib), MK-0371, AZD8477, AZ3146 and ARRY-520.

The last-named substances are so-called kinesin spindle protein inhibitors. Kinesin spindle protein (KSP, also known as Eg5, HsEg5, KNSL1 or KIF11) is a kinesin-like motor protein which is essential for the function of the bipolar mitotic spindle. Inhibition of KSP leads to mitotic arrest and, over a relatively long term, to apoptosis (Tao et al., Cancer Cell 2005 July 8(1), 39-59). Following the discovery of the first cell-penetrating KSP inhibitor, monastrol, KSP inhibitors became established as a class of novel chemotherapeutics (Mayer et al., Science 286: 971-974, 1999) and are the subject matter of a number of patents (e.g., WO2006/044825; WO2005/051922; WO2006/060737; WO03/060064; WO03/040979 and WO03/049527). However, since KSP is only active for a brief period during the mitosis phase, KSP inhibitors must be present in sufficiently high concentrations during this phase. ADCs with certain KSP inhibitors are disclosed in WO2014/151030.

In addition, ADCs with imidazole KSP inhibitors differing structurally from the KSP inhibitors of the ADCs described here are known from WO2006/002236, WO2007/021794 and WO2008/086122.

Furthermore, imidazole and benzimidazole derivatives are known as active compounds from U.S. Pat. No. 7,662,581 B1.

Imidazole, oxazole and diazepine derivatives are also described as active compounds in WO2004/100873.

The present invention relates to ADCs with pyrrole and pyrazole KSP inhibitors.

In WO2015/096982 and in WO2016/096610, ADCs with KSP inhibitors which also comprise enzymatically cleavable linkers and have a corresponding activity profile are disclosed.

However, it is desirable to obtain a distinctly better activity profile and/or exhibit improved properties.

It is therefore the object of the invention to provide new binder/active agent conjugates, particularly ADCs with KSP inhibitors and enzymatically cleavable linkers having an improved activity profile and/or improved properties.

Legumain is a tumor-associated asparaginyl endopeptidase (S. Ishii, Methods Enzymol. 1994, 244, 604; J. M. Chen et al. J. Biol. Chem. 1997, 272, 8090) and was used for processing prodrugs of small cytotoxic molecules, for example, of doxorubicin and etoposide derivatives among others (W. Wu et al. Cancer Res. 20 2006, 66, 970; L. Stem et al. Bioconjugate Chem. 2009, 20, 500; K. M. Bajjuri et al. ChemMedChem 2011, 6, 54).

Other lysosomal enzymes are, for example, cathepsin or glycosidases for example β-glucuronidases, which have also been used for releasing active compounds by enzymatic dissociation of prodrugs. Groups enzymatically cleavable in vivo are especially 2-8-oligopeptide groups or glycosides. Peptide cleavage sites are disclosed in Bioconjugate Chem. 2002, 13, 855-869, in Bioorganic & Medicinal Chemistry Letters 8 (1998) 3341-3346 and in Bioconjugate Chem. 1998, 9, 618-626. These include, for example, valine-alanine, valine-lysine, valine-citrulline, alanine-lysine and phenylalanine-lysine (optionally with additional amide group).

### SUMMARY OF THE INVENTION

Various antibody-drug conjugates (ADCs) with enzymatically cleavable linkers have been described in the prior art, but their activity profiles are not optimal. For example, it would be desirable to have available ADCs that exhibit a broader efficacy on different cells. In addition, such ADCs should also have good activity with simultaneously lower active compound concentrations and improved properties.

Thus it is an object of the present invention to provide more effective compounds which after administration at a relatively low concentration, exhibit long-lasting apoptotic action and are thus useful in cancer therapy. On one hand, the profile of the metabolites released intracellularly from the ADCs play a decisive role. Frequently the metabolites formed from ADCs are substrates of efflux pumps and/or have high permeability through cell membranes. Both phenomena may contribute to a short residence time and thus to suboptimal apoptotic action in the tumor cell.

Therefore the subject of the present invention is binder/active agent conjugates, particularly ADCs with a specific active agent (toxophore)-linker-antibody composition, which have a particularly interesting activity profile with respect to potency and activity spectrum. To further improve the tumor selectivity of ADCs and their metabolites, the ADCs were provided with peptide linkers that can be cleaved by lysosomal tumor-associated enzymes such as legumain and thus release the metabolite (toxophore).

Suitable antibodies are, for example, antibodies selected from the group of CXCR5 antibodies.

Thus the tumor selectivity is determined not only by the selection of the antibody, but additionally by the enzymatic dissociation of the peptide derivative, e.g., by tumor-associated enzymes such as legumain. The metabolites released by the ADCs according to the invention in the tumor cells are also characterized by a particularly interesting property profile. They also exhibit low efflux from the tumor cell, leading to high exposure to the active agent in tumors.

Thus a high activity in the tumor cell is achieved, whereas because of the poor permeability, only a low systemic cytotoxic activity exists, resulting in low off-target toxicity.

The kinesin spindle protein inhibitors used om the ADCs according to the invention have an amino group that is essential to the effect. By modifying this amino group with peptide derivatives, the effect with respect to the kinesin spindle protein is blocked, and thus the development of a cytotoxic effect is also inhibited. These peptide derivatives may also be components of the linker to the antibody. However, if this peptide residue or the peptide linker can be released from the active agent by tumor-associated enzymes such as legumain, the effect can be re-established in the tumor tissue in a controlled manner. The particular property profile of the metabolites formed in the tumor is guaranteed by a further modification of the kinesin spindle protein inhibitor at a different position from the amino group in the molecule, but this does not impair the high potency at the target.

In addition, the ADCs according to the invention allow high loading of the antibody (called DAR, Drug-to-Antibody ratio), which surprisingly does not negatively affect the physicochemical and pharmacokinetic behavior of the ADCs compared with the unconjugated antibody.

Surprisingly, it has now been found that antibody-active agent conjugates of general formula (I)

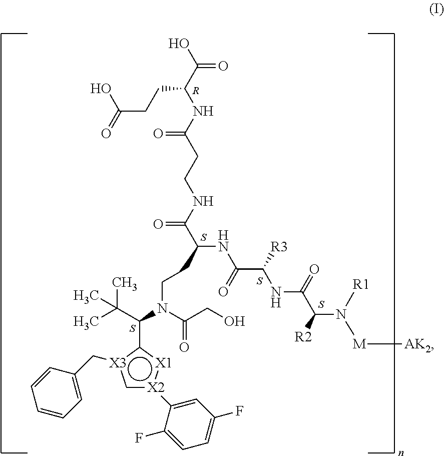

in which
X₁ represents N,
X₂ represents N and
X₃ represents C;
   or
X₁ represents CH or CF,
X₂ represents C and
X₃ represents N;
   or
X₁ represents NH,
X₂ represents C and
   X₃ represents C;
      or
   X₁ represents CH,
   X₂ represents N and
   X₃ represents C.
   R¹ represents hydrogen or methyl,
   R² represents methyl, ethyl, —CH₂—CH(CH₃)₂, —CH₂—C(═O)OH or isopropyl;
   R³ represents methyl, ethyl, —CH₂—CH(CH₃)₂ or —CH₂—C(═O)—NH₂,
   M represents the group
      #—C(═O)—CH(CH₃)—NH—C(═O)—(CH₂)₂₋₈—C(═O)-### or
      #—C(═O)— (CH₂)₃—C(═O)-###,
      Stands,
n represents a number from 1 to 50,
AK2 represents a binder or a derivative thereof, preferably an antibody or an antigen-binding fragment,
# represents the bond to the active agent and
### represents the bond to an N atom of a lysine side chain of the binder, and their salts, solvates and salts of these solvates, which have superior properties compared to the conjugates known from the prior art.

Preference is given to those binder/active agent conjugates of the formula (I)

in which
X₁ represents CH,
X₂ represents C and
X₃ represents N;
R¹ represents hydrogen or methyl,
R² represents methyl, —CH₂—CH(CH₃)₂, —CH₂—C(═O)OH or isopropyl,
R³ represents methyl, ethyl, —CH₂—CH(CH₃)₂ or —CH₂—C(═O)—NH₂,
M represents the group
   #—C(═O)—CH(CH₃)—NH—C(═O)—(CH₂)³—C(═O)-### or
   #—C(═O)— (CH₂)₃—C(═O)-###,
n represents a number from 1 to 50,
AK₂ represents a binder or a derivative thereof, preferably represents an antibody or an antigen-binding fragment,
# represents the bond to the active agent and
### represents the bond to an N-atom of a lysine side chain of the binder,
   and their salts, solvates and salts of these solvates.

Particularly preferred are those binder/active agent conjugates of formula (I),

in which
X₁ represents CH,
X₂ represents C and
X₃ represents N;
R¹ represents hydrogen or methyl,
R² represents methyl or isopropyl,
R³ represents methyl or —CH₂—C(═O)—NH₂,
M represents the group
   #—C(═O)—CH(CH₃)—NH—C(═O)—(CH₂)₃—C(═O)-###,
n represents a number from 1 to 50,
AK₂ represents a binder or a derivative thereof, preferably represents an antibody or an antigen-binding fragment,
# represents the bond to the active agent and
### represents the bond to an N-atom of a lysine side chain of the binder, and their salts, solvates and salts of these solvates.

Very particularly preferred are those binder/active agent conjugates formula (I) in which
X₁ represents CH,
X₂ represents C and
X₃ represents N;
R¹ represents methyl
R² represents methyl,
R₃ represents —CH₂—C(═O)—NH₂,
M represents the group
   #—C(═O)—CH(CH₃)—NH—C(═O)—(CH₂)₃—C(═O)-###,
n represents a number from 1 to 50,
AK₂ represents a binder or a derivative thereof, preferably represents an antibody or an antigen-binding fragment,
# represents the bond to the active agent and
### represents the bond to an N-atom of a lysine side chain of the binder,
   and their salts, solvates and salts of these solvates.

Particularly preferred are those binder/active agent conjugates of formula (I),
in which
R₁ represents methyl, represents methyl,
R₂
R₃ represents —CH₂—C(═O)—NH₂,
M represents the group
   #—C(═O)—CH(CH₃)—NH—C(═O)—(CH₂)₃—C(═O)-###,
n represents a number from 1 to 20 and
AK₂ represents an antibody or represents an antigen-binding antibody fragment thereof,
   # represents the bond to the active agent and
   ### represents the bond to a N-atom of a lysine side chain of the antibody or the antigen-binding antibody fragment thereof,
   as well as their salts, solvates and salts of these solvates.

Selected are those binder/active agent conjugates of formula (I) according to the structure

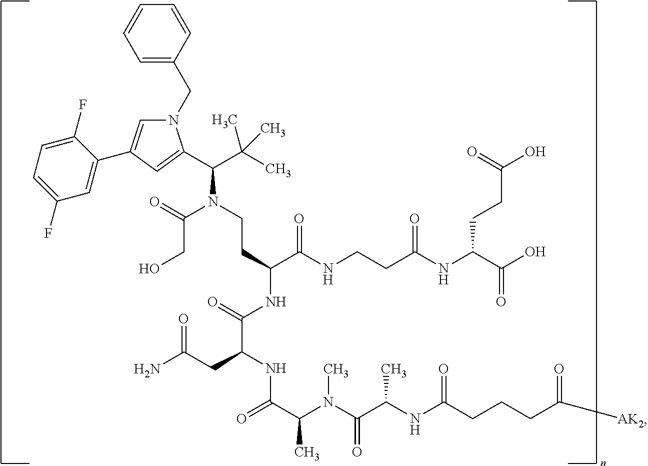

in which
AK₂ represents an antibody linked over a N-atom of a lysine side chain and
n is from 1 to 50,
   as well as their salts, solvates and salts of these solvates.

Preferred among these are those binder/active agent conjugates in which

n is 1 to 20,
as well as their salts, solvates and salts of these solvates.

Those binder/active agent conjugates are also preferred in which

n is 1 to 8,
as well as their salts, solvates and salts of these solvates.

Also preferred are those binder/active agent conjugates in which

n is 4 to 8,
as well as their salts, solvates and salts of these solvates.

Preferred are those binder/active agent conjugates of the formulas mentioned above in which AK₂ represents a binder that binds specifically to an extracellular cancer target molecule. In a preferred embodiment the binder, after binding to its extracellular target molecule on the target cell, is internalized by the target cell through the binding. Preferably the binder is an antibody or an antigen-binding fragment.

In a preferred subject of the invention the extracellular cancer target molecule is selected from the group consisting of the cancer target molecules CXCR5.

In a preferred subject of the invention the binder AK₂ is an anti-CXCR5 antibody or an antigen-binding antibody fragment thereof,

Preferred are those binder/active agent conjugates of the formulas mentioned above in which AK₂ represents an antibody selected from the group consisting of TPP-10063, TPP-14511, TPP-14509, TPP-14499, TPP-14505, TPP-14514 and TPP-14495, or an antigen-binding fragment thereof. Particularly preferred are those binder/active agent conjugates of the formulas mentioned in which AK₂ represents an antibody selected from the group consisting of TPP-14511, TPP-14509, TPP-14499, TPP-14505, TPP-14514 and TPP-14495, or for an antigen-binding fragment thereof.

### DESCRIPTION OF THE FIGURES

FIG. 1: Sequence listing of sequences of antibodies for binder/active agent conjugates and of sequences of the target proteins.

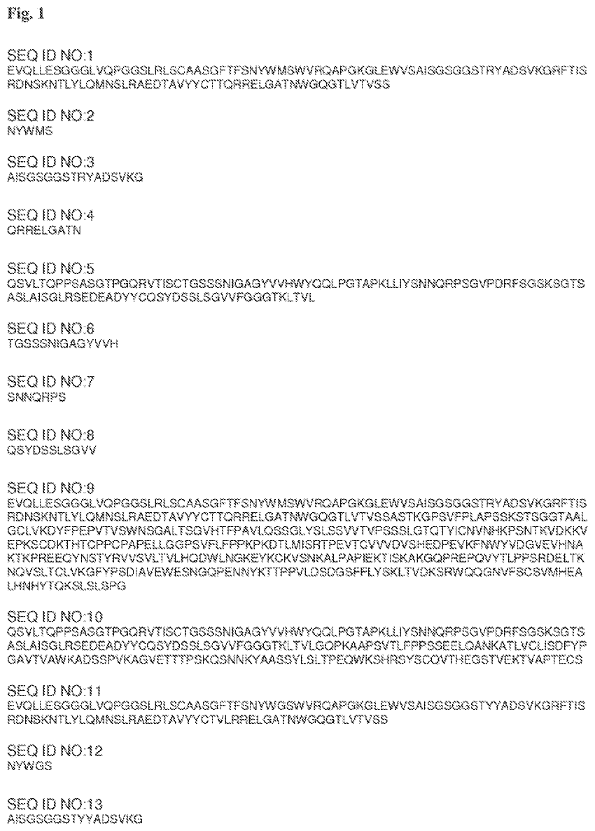

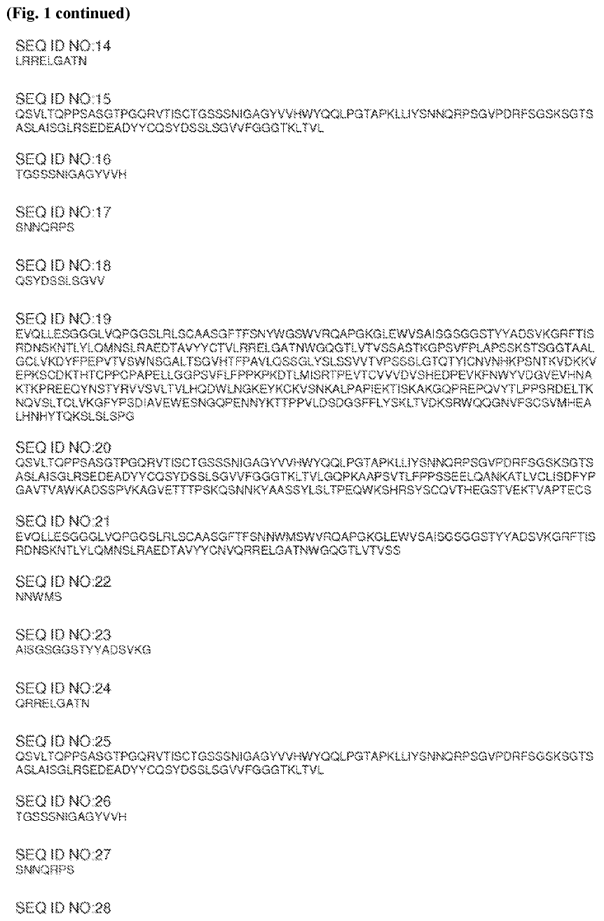

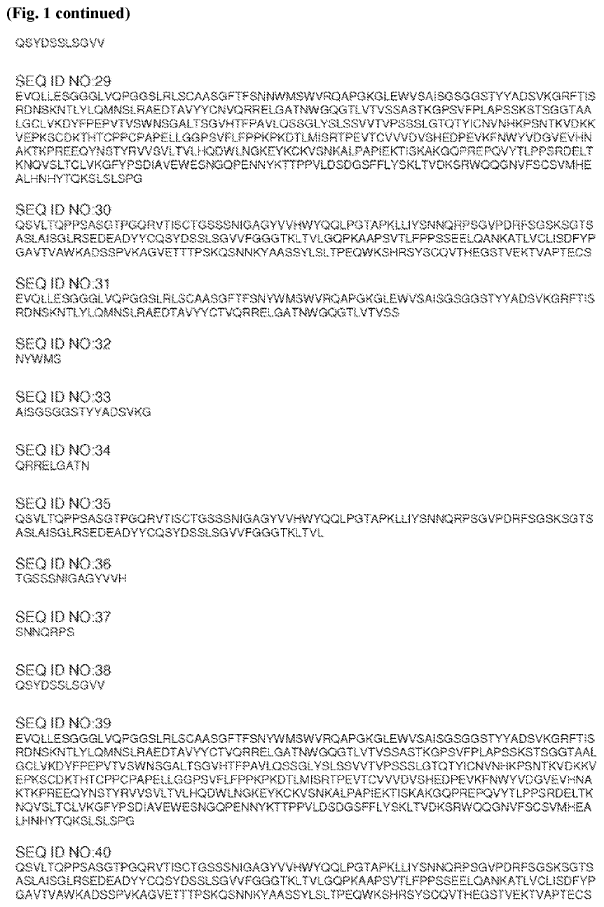

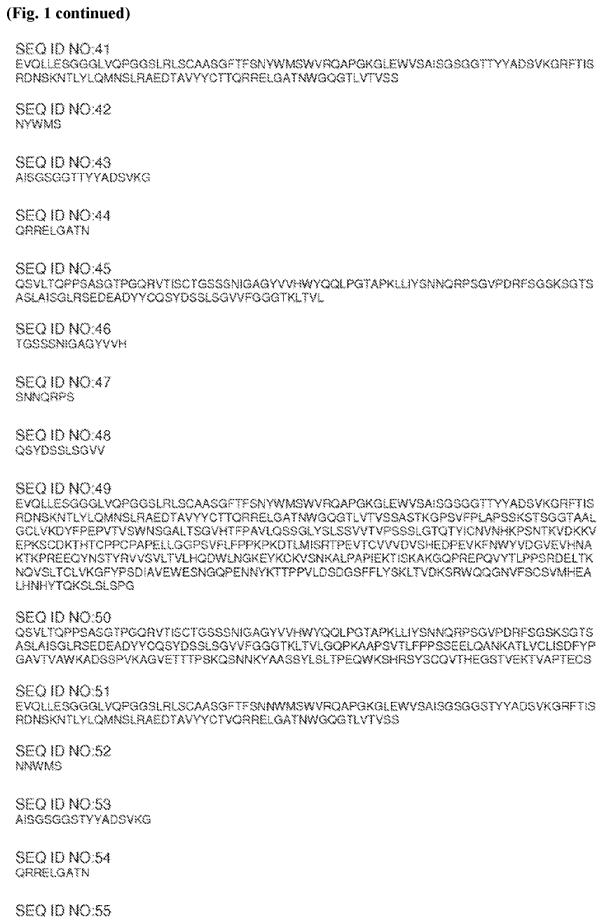

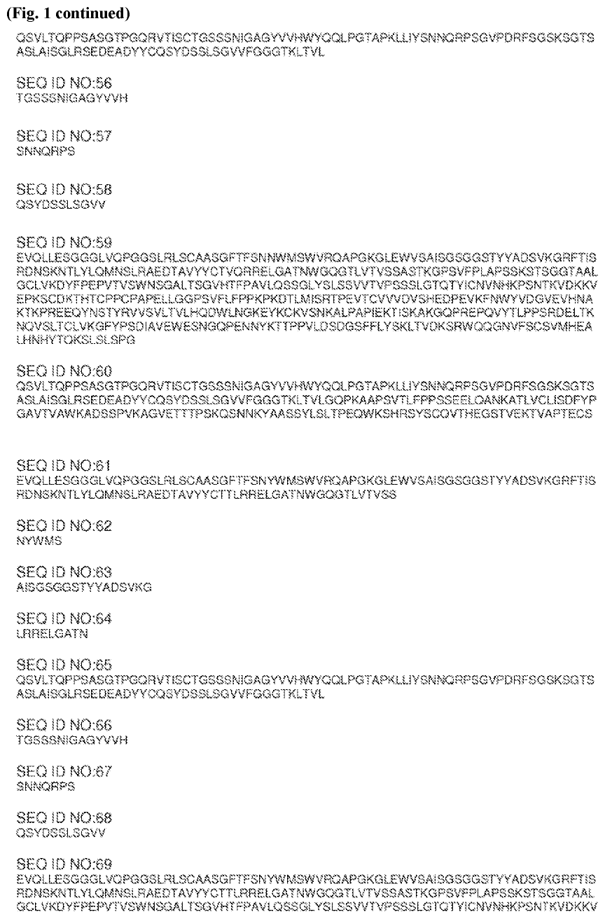

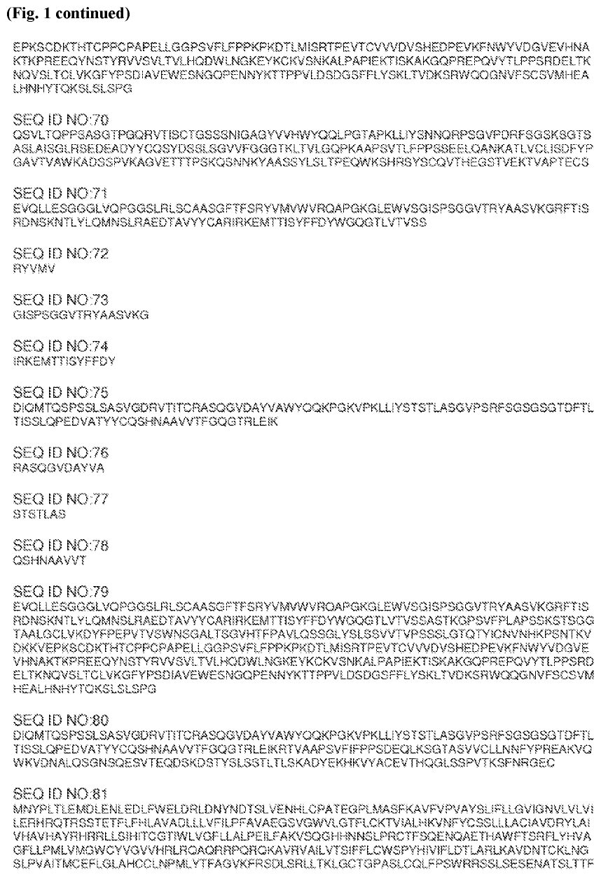

### DETAILED DESCRIPTION OF THE INVENTION

The invention provides conjugates of a binder or derivatives thereof with one or more active agent molecules, wherein the active agent molecule is a kinesin spindle protein inhibitor (KSP inhibitor).

In the following, usable binders according to the invention, usable KSP inhibitors thereof according to the invention and usable linkers according to the invention that can be used in combination without limitation will be described. In particular, the binders presented as preferred or particularly preferred can be used in combination with the KSP inhibitors presented as preferred or particularly preferred, optionally in combination with the respective linkers presented as preferred or particularly preferred.

### Particularly Preferred KSP-Inhibitor Conjugates (Binder/Active Agent Conjugates)

Particularly preferred according to the invention are the following KSP-inhibitor conjugates, wherein AK₂ represents binders or a derivative thereof (preferably an antibody), and n resents a number from 1 to 50, preferably 1 to 20, preferably 1 to 8, especially preferably 4 to 8. AK₂ preferably represents an antibody bonded via a lysine residue to the KSP inhibitor Binders or antibodies used here are preferably the binders and antibodies described as preferred in the description.

Particular preference is given to the following binder/active agent-conjugates:

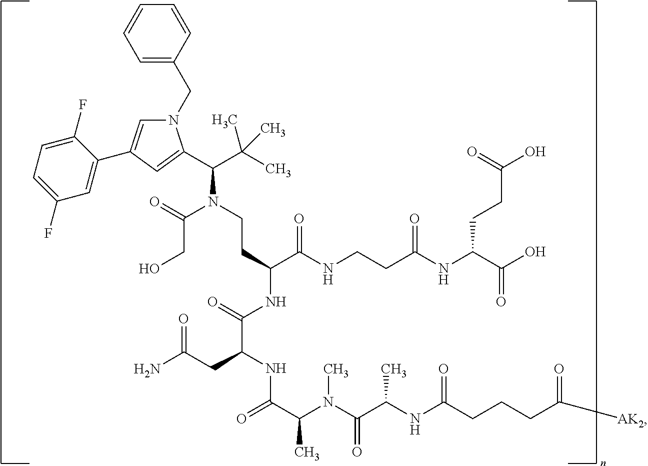

Particular preference is given to those binder/active agent conjugates of the formulas presented in which AK₂ represents a binder that binds specifically to an extracellular cancer target molecule. In a preferred embodiment, the binder, after binding to its extracellular target molecule on the target cell, is internalized by the target cell through the binding.

In a preferred subject of the invention, the extracellular cancer target molecule is selected from the group consisting of the cancer target molecules, particularly CXCR5.

In a preferred subject of the invention, the binder AK₂ is an anti-CXCR5 antibody or an antigen-binding antibody fragment thereof.

Preferred are those binder/active agent conjugates of the formulas mentioned in which AK₂ represents an antibody selected from the group consisting of TPP-10063, TPP-14511, TPP-14509, TPP-14499, TPP-14505, TPP-14514 and TPP-14495, or represents an antigen-binding fragment thereof. Particularly preferred are those binder/active agent conjugates of the formulas mentioned in which AK₂ represents an antibody selected from the group consisting of TPP-14511, TPP-14509, TPP-14499, TPP-14505, TPP-14514 and TPP-14495 or an antigen-binding fragment thereof.

Accordingly, especially preferred binder/active agent conjugates are those of formula (I), in which
R¹ represents methyl,
R² represents methyl,
R₃ represents —CH₂—C(═O)—NH₂,
M represents the group
#—C(═O)—CH(CH₃)—NH—C(═O)—(CH₂)₃—C(═O)-###,
n represents a number from 1 to 20 and represents an anti-CXCR5 antibody selected from the group consisting of TPP-14511, TPP-14509, TPP-14499, TPP-14505, TPP-14514 and TPP-14495, or
AK₂ represents an antigen-binding antibody fragment thereof,
# represents the bond to the active compound and
### represents the bond to an N-atom of a lysine side chain of the antibody or the antigen-binding antibody fragment thereof,
   as well as their salts, solvates and salts of these solvates.
      KSP inhibitor—linker intermediates and preparation of the coniugates

The conjugates according to the invention are prepared in that first the low molecular weight KSP inhibitor thereof is provided with a linker. The intermediate prepared in this way is then reacted with the binder (preferably antibody).

For an intermediate coupled with a lysine radical and the subsequent coupling with the antibody, the reaction can be illustrated as follows:

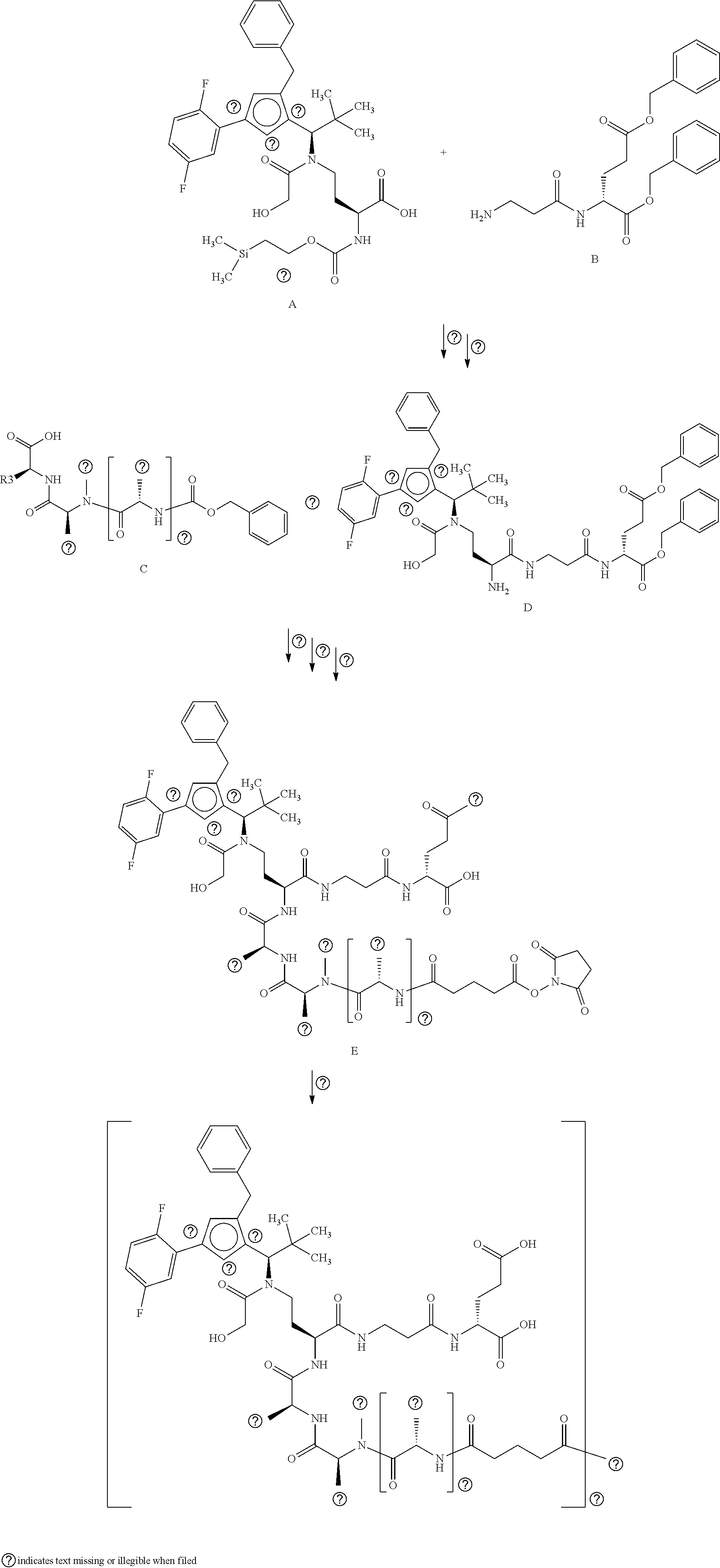

In the above reaction scheme, X₁, X₂, X₃, R¹, R², R³ and AK₂ have the meanings given in formula (I) and here R⁴ represents methyl and m is 0 or 1.

The synthesis of building block A is described in WO2015/096982. The peptide derivatives B and C were prepared by classical methods of peptide chemistry. The intermediates C and D were coupled using HATU in DMF in the presence of N, N-diisopropylethylamine at RT. Then both the benzyloxycarbonyl protective group and the benzyl were split off by hydrogenolysis over 10% palladium on active carbon. The completely deprotected intermediate was then reacted with 1,1′-[(1,5-dioxopentan-1,5-diyl)bis(oxy)]dipyrrolidine-2,5-dione in DMF in the presence of N,N-diisopropylethylamine at RT to form the ADC precursor molecule E. This active ester was then coupled with the corresponding antibodies as described in Chapter B-4.

In the above reaction scheme, X₁, X₂, X₃, R¹, R², R³ and AK₂ have the meanings given in formula (I) and here R⁴ represents methyl and n is 1.

Using an analogous procedure, compounds in which m represents 0 may also be prepared.

### Binders

The term “binder” is understood in the broadest sense to mean a molecule which binds with a target molecule present in a certain population to be addressed with the binder/active agent conjugate. The term binder is to be understood in its broadest meaning and also comprises, for example, lectins, proteins capable of binding to certain sugar chains, or phospholipid-binding proteins. Such binders include, for example, high-molecular-weight proteins (binding proteins), polypeptides or peptides (binding peptides), non-peptidic (e.g., aptamers (U.S. Pat. No. 5,270,163) review article by Keefe AD., et al., Nat. Rev. Drug Discov. 2010; 9:537-550), or vitamins) and all other cell-binding molecules or substances. Binding proteins are e.g., antibodies and antibody fragments or antibody mimetics such as affibodies, adnectins, anticalins, DARPins, avimers, nanobodies (review article by Gebauer M. et al., Curr. Opinion in Chem. Biol. 2009; 13:245-255; Nuttall S. D. et al., Curr. Opinion in Pharmacology 2008; 8:608-617). Binding peptides are, for example, ligands of a ligand-receptor pair, such as VEGF of the ligand-receptor pair VEGF/KDR, such as transferrin of the ligand-receptor pair transferrin/transferrin receptor or cytokine/cytokine receptor, such as TNFalpha of the ligand-receptor pair TNFalpha/TNFalpha receptor.

The binder may be a binding protein. Preferred embodiments of the binder are an antibody, an antigen-binding antibody fragment, a multispecific antibody or an antibody mimetic.

Various possibilities are also known from the literature for covalent coupling (conjugation) of organic molecules to binders and particularly antibodies. According to the invention, preference is given to the conjugation of the toxophore to the antibody over one or more sulfur atoms of cysteine residues of the antibody and/or over one or more NH groups of lysine residues of the antibody. However, it is also possible to bind the toxophore to the antibody via free carboxyl groups or via sugar residues of the antibody.

A “target molecule” is understood in the broadest sense to mean a molecule that is present in the target cell, and may be a protein (for example, a receptor of a growth factor) or a non-peptidic molecule (for example, a sugar or a phospholipid. Preferably it is a receptor or an antigen.

The term “extracellular” target molecule describes a target molecule, bound to a cell, which is located outside of the cell or the part of a target molecule which is located outside of a cell, i.e., a binder may bind to an intact cell to its extracellular target molecule. An extracellular target molecule may be anchored in the cell membrane or may be a component of the cell membrane.

The person skilled in the art is aware of methods for identifying extracellular target molecules. For proteins, this may take place by determining the transmembrane domain(s) and the orientation of the protein in the membrane. This information is usually deposited in protein databases (e.g., SwissProt).

The term “cancer target molecule” describes a target molecule which is present in increased quantities on one or more species of cancer cells than on non-cancer cells of the same tissue type. Preferably, the cancer target molecule is selectively present on one or more cancer cell species compared with non-cancer cells of the same tissue type, where selective describes an at least two-fold enrichment on cancer cells compared to non-cancer cells of the same tissue type (a “selective cancer target molecule”). The use of cancer target molecules permits the selective therapy of cancer cells using the conjugates to the invention.

The binder can be attached to the linker via a bond. The binder can be linked via a heteroatom of the binder. Heteroatoms of the binder according to the invention that can be used for linking are sulfur (in one embodiment via a sulfhydryl group of the binder), oxygen (according to the invention by way of a carboxyl or hydroxyl group of the binder) and nitrogen (in one embodiment via a primary or secondary amine group or amide group of the binder). These heteroatoms may be present in the natural binder or be introduced by chemical or molecular biological methods. According to the invention, the attachment of the binder to the toxophore has only a slight influence on the binding activity of the binder to the target molecule. In a preferred embodiment, the linkage has no effect on the binding activity of the binder to the target molecule.

The term “antibody” according to the present invention is to be understood in its broadest meaning and comprises immunoglobulin molecules, for example intact or modified monoclonal antibodies, polyclonal antibodies or multispecific antibodies (e.g., bispecific antibodies). An immunoglobulin molecule preferably comprises a molecule having four polypeptide chains, two heavy chains (H chains) and two light chains (L chains), which are typically linked by disulfide bridges. Each heavy chain comprises one variable domain of the heavy chain (abbreviated as VH) and a constant domain of the heavy chain. For example, the constant domain of the heavy chain may comprise three domains CH1, CH2 and CH3. Each light chain comprises one variable domain (abbreviated as VL) and a constant domain. The constant domain of the light chain comprises one domain (abbreviated as CL). The VH and VL domains can be further subdivided into regions with hypervariability, also called complementarity determining regions (abbreviated as CDR) and regions with lower sequence variability “framework region,” abbreviated as FR). Each VH and VL region is typically made of three CDRs and up to four FRs, for example, from the amino terminus to the carboxy terminus in of the following order: FR1, CDR1, FR2, CDR2, FR3, CDR3, FR4. An antibody can be obtained from each species suitable for this, e.g., rabbit, llama, came, mouse or rat. In one embodiment the antibody is of human or murine origin. For example, an antibody can be human, humanized or chimeric.

The term “monoclonal” antibody designates antibodies obtained from a population of substantially homogeneous antibodies, i.e., individual antibodies of the population are identical except for naturally occurring mutations, which may be present in small numbers. Monoclonal antibodies recognize a single antigen binding site with high specificity. The term monoclonal antibody does not refer to a particular manufacturing process.

The term “intact” antibody refers to antibodies which comprise both an antigen-binding domain and the constant domain of the light and heavy chains. The constant domain can be a naturally occurring domain or a variant thereof in which several multiple amino acid positions were modified, and may also be glycosylated.

The term “modified intact” antibody refers to intact antibodies fused via their amino terminus or carboxy terminus by means of a covalent bond (e.g., a peptide bond) with an additional polypeptide or protein not originating from an antibody. In addition, antibodies may be modified such that reactive cysteines are introduced at defined positions to facilitate coupling to a toxophore (see Junutula et al. Nat Biotechnol. 2008 August; 26(8):925-32).

“Amino acid modification” or “mutation” here designates an amino acid substitution, insertion and/or deletion in a polypeptide sequence. The preferred amino acid modification here is a substitution. “amino acid substitution” or “substitution” here means replacement of an amino acid at a given position in a protein sequence by another amino acid. For example, the substitution Y50W describes a variant of a parent polypeptide in which the tyrosine at position 50 is replaced by a tryptophan. A “variant” of a polypeptide describes a polypeptide having an amino acid sequence substantially identical to a reference polypeptide, typically a native or “parent” polypeptide. The polypeptide variant may have one or more amino acid exchanges, deletions and/or insertions at particular positions in the native amino acid sequence.

The term “human” antibody refers to antibodies that can be obtained from a human or that are synthetic human antibodies. A “synthetic” human antibody is an antibody which can be obtained partially or with difficulty entirely from synthetic sequences in silico, based on the analysis of human antibody sequences. For example, a human antibody can be encoded by a nucleic acid isolated from a library of antibody sequences of human origin. One example of such an antibody can be found is in Soderlind et al., Nature Biotech. 2000, 18:853-856. Such “human” and “synthetic” antibodies also include aglycosylated variants obtained either by deglycosylation with PNGaseF or by mutation from N297 (Kabat numbering) of the heavy chain to any other amino acid.

The term “humanized” or “chimeric” antibody describes antibodies consisting of a non-human and a human sequence portion. In these antibodies part of the sequence of the human immunoglobulin (recipient) is replaced by sequence portions of a non-human immunoglobulin (donor). In many cases the donor is a murine immunoglobulin. In humanized antibodies amino acids of the CDR of the recipient are replaced by amino acids of the donor. Sometimes the amino acids of the framework are also replaced by the corresponding amino acids of the donor.

In some cases the humanized antibody contains amino acids that were not present either in the recipient nor n the donor and that were introduced during the optimization of the antibody. In chimeric antibodies the variable domains of the donor immunoglobulin are fused with the constant regions of a human antibody. Such “humanized” and “chimeric” antibodies also include aglycosylated variants produced either by deglycosylation by PNGaseF or by mutation von N297 (Kabat numbering) of the heavy chain to any other amino acid.

The term complementarity-determining region (CDR) as used here relates to the amino acids of a variable antibody domain that are required for binding to the antigen. Each variable region typically has three CDR regions, which are designated CDR1, CDR2 und CDR3. Each CDR region can comprise amino acids according to the definition of Kabat and/or amino acids of a hypervariable loops defined according to Chotia. The definition according to Kabat, for example, comprises the region of approximately amino acid position 24-34 (CDR1), 50-56 (CDR2) und 89-97 (CDR3) of the variable light chain/domain (VL) and 31-35 (CDR1), 50-65 (CDR2) and 95-102 (CDR3) of the variable heavy chain/domain (VH) (Kabat et al., Sequences of Proteins of Immunological Interest, 5th Ed. Public Health Service, National Institutes of Health, Bethesda, Md. (1991)). For example, the definition according to Chotia comprises the region from approximately amino acid position 26-32 (CDR1), 50-52 (CDR2) und 91-96 (CDR3) of the variable light chain (VL) and 26-32 (CDR1), 53-55 (CDR2) and 96-101 (CDR3) of the variable heavy chain (VH) Chothia and Lesk; J Mol Biol 196: 901-917 (1987)). In some cases, a CDR may comprise amino acids from a CDR region defined according to Kabat and Chotia.

Antibodies may be categorized into different classes depending on the amino acid sequence of the constant domain of the heavy chain. There are five main classes of intact antibodies: IgA, IgD, IgE, IgG and IgM, wherein several of them can be subdivided into additional subclasses. (isotypes), e.g., IgG1, IgG2, IgG3, IgG4, IgA1 and IgA2. The constant domains of the heavy chain, which correspond to the different classes, are designated as [alpha/a], [delta/6], [epsilon/ε], [gamma/γ] and [my/μ]. Both the three-dimensional structure and the subunit structure of antibodies are known.

The term “functional fragment” or “antigen-binding antibody fragment” of an antibody/immunoglobulin is defined as a fragment of an antibody/immunoglobulin (e.g., the variable domains of an IgG), which still comprise the antigen-binding domains of the antibody/immunoglobulin. The “antigen-binding domain” of an antibody typically comprises one or more hypervariable regions of an antibody, e.g., the CDR, CDR2 and/or CDR3 region. However, the “framework” or the “skeleton” region of an antibody may also play a role in binding the antibody to the antigen. The framework region forms the skeleton of the CDRs. Preferably the antigen-binding domain comprises as least amino acids 4 to 103 of the variable light chain and amino acids 5 to 109 of the variable heavy chain, more preferably amino acids 3 to 107 of the variable light chain and 4 to 111 of the variable heavy chain, especially preferably the complete variable light and heavy chains, thus amino acids 1-109 of the VL and 1 to 113 of the VH (numbering according to WO97/08320).

“Functional fragments” or “antigen-binding antibody fragments” of the invention non-exclusively comprise Fab, Fab′, F(ab)₂ und Fv fragments, diabodies, single domain antibodies (DAbs), linear antibodies, single-chain antibodies (single-chain Fv, abbreviated as scFv); and multispecific, antibodies, e.g., bi- and tri-specific, antibodies, formed from antibody fragments. C. A. K. Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). Antibodies other than “multi-specific”or “multifunctional” are those with identical binding sites. Multispecific antibodies can be specific for various epitopes of an antigen or specific for epitopes of more than one antigen (see, e.g., WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; or Kostelny et al., 1992, J. Immunol. 148: 1547 1553). An F(ab′)2 or Fab molecule can be constructed such that the number of intermolecular disulfide interactions occurring between the Chl and the CL domains can be reduced or completely prevented.

“Epitopes” refer to protein determinants that can undergo specific binding with an immunoglobulin or T-cell receptors. Epitopic determinants normally consist of chemically active surface groups of molecules such as amino acids or sugar side chains or combinations thereof, and normally have specific 3-dimensional structural characteristics as well as specific charge characteristics.

“Functional fragments” or “antigen-binding antibody fragments” can be fused with an additional polypeptide or protein, not originating from an antibody, via its amino terminus or carboxy terminus through a covalent bond (e.g., a peptide bond). In addition, antibodies and antigen-binding fragments can be modified by introducing reactive cysteines at defined locations to facilitate coupling to a toxophore (see Junutula et al. Nat Biotechnol. 2008 August; 26(8):925-32).

Polyclonal antibodies can be prepared by methods known to a person with ordinary skill in the art. Monoclonal antibodies can be prepared by methods known to a person with ordinary skill in the art (Kohler und Milstein, Nature, 256, 495-497, 1975). Human or humanized monoclonal antibodies can be prepared by methods known to a person with ordinary skill in the art (Olsson et al., Meth Enzymol. 92, 3-16 or Cabilly et al U.S. Pat. No. 4,816,567 or Boss et al U.S. Pat. No. 4,816,397).

A person skilled in the art is aware of various methods for producing human antibodies and fragments, for example using transgenic mouse (N Lonberg und D Huszar, Int Rev Immunol. 1995; 13(1):65-93) or Phage Display Technologies (August 15; 352(6336):624-8). Antibodies of the invention can be obtained from recombinant antibody libraries containing, for example, amino acid sequences of a multiplicity of antibodies compiled from a large number of healthy volunteers. Antibodies can also be prepared using known recombinant DNA. The nucleic acid sequence of an antibody can be determined by routine sequencing or obtained from publicly available databases.

An “isolated” antibody or binder has been purified to remove other constituents of the cell. Contaminating constituents of a cell that can interfere with diagnostic or therapeutic use thereof are, for example, enzymes, hormones, or other peptidic or non-peptidic constituents of a cell.

A preferred antibody or binder is one that has been purified to the extent of more than 95% by weight based on the antibody or binder (determined by, e.g., the Lowry method, UV-Vis spectroscopy or by SDS capillary gel electrophoresis). Additionally an antibody that has been purified to such an extent that it is possible to determine at least 15 amino acids from the amino terminus or an internal amino acid sequence or was purified to homogeneity, wherein the homogeneity is determined by SDS-PAGE under reducing or non-reducing conditions (the detection can be carried out by Coomassie Blue staining or preferably by silver staining). However, an antibody is normally prepared by one or more purification steps.

The term “specific binding” or “binds specifically” relates to an antibody or binder that binds to a predetermined antigen/target molecule. Specific binding of an antibody or binder typically describes an antibody or binder with an affinity of at least 10⁻⁷ M (as Kd value; thus preferably those with Kd values smaller than 10⁻⁷ M), wherein the antibody or binder has an at least two-fold higher affinity for the predetermined antigen/target molecule than to a nonspecific antigen/target molecule (e.g., bovine serum albumin or casein) that is not the predetermined antigen/target molecule or a closely related antigen/target molecule. Specific binding of an antibody or binder does not rule out the possibility of the antibody or binder binding to multiple antigens/target molecules (e.g., orthologs from various species). The antibodies referred to have an affinity of at least 10⁻⁷ M (as Kd value; thus preferably those with Kd values of less than 10⁻⁷ M), preferably of at least 10⁻⁸ M, especially preferably in the range of 10⁻⁹ M to 10⁻¹¹ M. The Kd values can be determined, e.g., by surface plasmon resonance spectroscopy.

The antibody-active agent conjugates according to the invention likewise have affinities in these ranges. Preferably the affinity is not substantially affected by the conjugation of the active agents (the affinity is generally reduced by less than one order of magnitude, thus e.g., at most from 10⁻⁸ M to 10⁻⁷ M).

Furthermore, the antibodies used according to the invention are preferably characterized by high selectivity. High selectivity is present when the antibody according to the invention has a better affinity for the target protein by at least a factor of 2, preferably a factor of 5 or particularly preferably a factor of 10 than for an unrelated other antigen, e.g., human serum albumin (the affinity can be determined, e.g., by surface plasmon resonance spectroscopy).

In addition, the antibodies used according to the invention are preferably cross-reactive. To facilitate preclinical studies, for example toxicology or efficacy studies (e.g., in xenograft mice) and to interpret them more clearly, it is advantageous if the antibody used according to the invention not only binds the human target protein, but also binds the species target protein of the species used in the species used for the studies. In one embodiment the antibody used according to the invention, in addition to the human target protein, is cross-reactive with the target protein of at least one additional species. Species from the rodent, dog and non-human primate families are preferably used for toxicologic and efficacy studies. Preferred rodent species are mouse and rats. Preferred non-human primates are rhesus monkeys, chimpanzees and long-tailed macaques.

In one embodiment the antibody used according to the invention, in addition to the human target protein, is cross-reactive to the target protein of at least one additional species selected from the group of species consisting of mouse, rat and long-tailed macaque (Macaca fascicularis). Particularly preferred antibodies for use according to the invention are those which, in addition to the human target protein, are at least cross-reactive to the monkey target protein (e.g., chimpanzees). Preferred are cross-reactive antibodies, the affinity of which for the target protein of the other non-human species does not differ by more than a factor of 50, particularly not more than a factor of ten, from the affinity for the human target protein.

Antibodies Against a Cancer Target Molecule

Preferably the target molecule against which the binder, e.g., an antibody or antigen binding fragment thereof is directed, is a cancer target molecule. The term “cancer target molecule” describes a target molecule that is present on one or more types of cancer cells in larger quantities compared to non-cancer cells of the same tissue type. Preferably the cancer target molecule is selectively present on one or more cancer cell types compared to non-cancer cells of the same issue type, wherein selectively means a two-fold enrichment of cancer cells compared to non-cancer cells of the same tissue type (a “selective cancer target molecule”). The use of cancer target molecules allows the selective therapy of cancer cells with the conjugates according to the invention.

Antibodies that are specific against an antigen, e.g., a cancer cell antigen, can be prepared by a person skilled in the art using methods with which he or she is familiar (e.g., recombinant expression) or acquired commercially (e.g., from Merck KGaA, Germany). Examples of known commercially available antibodies in cancer therapy are Erbitux® (Cetuximab, Merck KGaA), Avastin® (Bevacizumab, Roche) and Herceptin® (Trastuzumab, Genentech). Trastuzumab is a recombinant humanized monoclonal antibody of the IgGlkappa type which binds the extracellular domain of the human epidermal growth receptor with high affinity in a cell-based assay (Kd=5 nM). The antibody is produced using recombinant technology in CHO cells. All of these antibodies can also be prepared as aglycosylated variants of this antibody, either by deglycosylation using PNGase F or by mutation of N297 (Kabat numbering) of the heavy chain to any amino acid.

In a preferred embodiment, the target molecule is a selective cancer target molecule.

In a particularly preferred embodiment, the target molecule is a protein.

Cancer target molecules are known to the person skilled in the art.

In a preferred subject of the invention the cancer target molecule is CXCR5 (CD185; SwissProt: P32302; NCBI-Gene ID 643, NCBI reference sequence: NP_001707.1).

In a preferred embodiment the binder, after binding to its extracellular target molecule on the target cell, is internalized by the target cell through the bond. This means that the binder/active agent conjugate, which can be an immunoconjugate or an ADC, is taken up by the target cell. Then the binder is processed, preferably intracellularly, preferably lysosomally.

In one embodiment the binder is a binder protein. In a preferred embodiment the binder is an antibody, an antigen-binding antibody fragment, a multispecific antibody or an antibody mimetic.

Preferred antibody mimetics are affibodies, adnectins, anticalins, DARPins, avimers, or nanobodies. Preferred multispecific antibodies are bispecific and trispecific antibodies.

In a preferred embodiment the binder is an antibody or an antigen-binding antibody fragment, more preferably an isolated antibody or an isolated antigen-binding antibody fragment.

Preferred antigen-binding antibody fragments are Fab, Fab′, F(ab′)2 and Fv fragments, diabodies, DAbs, linear antibodies and scFv. Particularly preferred are Fab, diabodies and scFv.

In a particularly preferred embodiment the binder is an antibody. Particularly preferred are monoclonal antibodies or antigen-binding antibody fragments thereof. Further particularly preferred are human, humanized or chimeric antibodies or antigen-binding antibody fragments thereof.

Antibodies or antigen-binding antibody fragments that bind the cancer target molecules can be prepared by a person skilled in the art using known processes, for example chemical synthesis or recombinant expression. Binders for cancer target molecules can be commercially acquired or can be prepared by a person skilled in the art using known processes, e.g., chemical synthesis or recombinant expression. Additional methods for preparing antibodies or antigen-binding antibody fragments are described in WO 2007/070538 (see page 22 “antibodies”). A person skilled in the art is aware that methods such as so-called phage display libraries (e.g., Morphosys HuCAL Gold) can be created and used for discovering antibodies or antigen-binding antibody fragments (see WO 2007/070538, page 24 ff and AK [antibody] example 1 on page 70, AK example 2 on page 72). Additional methods for preparing antibodies using DNA libraries from B cells, are described for example on page 26 (WO 2007/070538). Methods for humanizing antibodies are described on pages 30-32 of WO2007/070538 and in detail in Queen, et al., Proc. Natl. Acad. Sci. USA 86:10029-10033, 1989 or in WO 90/0786. In addition, the person skilled in the art is aware of processes for recombinant expression of proteins in general and of antibodies in particular (see e.g., in Berger and Kimmel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biolony, (F. M. Ausabel et al. [Eds.], Current Protocols, Green Publishing Associates, Inc./John Wiley & Sons, Inc.); Harlow et al., (Monoclonal Antibodies: A Laboratory Manual, Cold Spring Harbor Laboratory Press (19881, Paul [Ed.]); Fundamental Immunology, (Lippincott Williams & Wilkins (1998)); and Harlow, et al., (Using Antibodies: A Laboratory Manual, Cold Spring Harbor Laboratory Press (1998)). A person skilled in the art is aware of the corresponding vectors, promoters and signaling peptides necessary for expression of a proteins/antibody. Customary processes are also described in WO 2007/070538 on pages 41-45. Processes for preparing an IgG1 antibody are described e.g., in WO 2007/070538 in Example 6 on page 74 ff. Processes with which the internalization of an antibody after binding to its antigen can be determined are familiar to a person skilled in the art and are described, for example, in WO 2007/070538 on page 80. The person skilled in the art can use the process described in WO 2007/070538, which was used for preparing carboanhydrase IX (Mn) antibodies, analogously for preparing antibodies with other target molecule specificity.

### Bacterial Expression

The person skilled in the art is aware of the way in which antibodies, antigen-binding fragments thereof, or variants thereof can be prepared with the aid of bacterial expression.

Suitable expression vectors for bacterial expression of desired proteins are constructed by inserting a DNA sequence coding for the desired protein in the functional reading frame together with suitable translation initiation and translation termination signals and with a functional promoter. The vector comprises one or more phenotypically selectable markers and a replication origin to enable the retention of the vector and, if desired, the amplification thereof within the host. Suitable prokaryotic hosts for transformation comprise, but art not limited to, E. coli, Bacillus subtilis, Salmonella typhimurium and various species from the genera Pseudomonas, Streptomyces, and Staphylococcus. Bacterial vectors can, be based on, for example, bacteriophages, plasmids, or phagemids. These vectors can contain selectable markers and a bacterial replication origin derived from commercially available plasmids. Many commercially available plasmids contain typical elements of the well-known cloning vector pBR322 (ATCC 37017). In bacterial systems, a number of advantageous expression vectors may be selected based on the intended use of the protein to be expressed.

After transformation of a suitable host strain and growth of the host strain to an appropriate cell density, the selected promoter is de-repressed/induced by suitable means (e.g., temperature change or chemical induction), and the cells are cultured for an additional period. The cells are usually harvested by centrifugation, if necessary digested by physical means or with chemical agents, and the resulting crude extract is retained for further purification. Therefore a further embodiment of the present invention is an expression vector comprising a nucleic acid that encodes a novel antibody of the present invention.

Naturally, antibodies of the present invention or antigen-binding fragments thereof include naturally purified products, products originating from chemical synthesis, and products produced by recombinant technologies in prokaryotic hosts, for example E. coli, Bacillus subtilis, Salmonella typhimurium and various species from the genera Pseudomonas, Streptomyces, and Staphylococcus, preferably E. coli.

### Mammalian Cell Expression

The person skilled in the art is aware of the way in which antibodies, antigen-binding fragments thereof, or variants thereof can be produced with the aid of mammalian cell expression.

Preferred regulatory sequences for expression in mammalian cell hosts comprise viral elements that lead to high expression in mammalian cells, such as promoters and/or expression amplifiers derived from cytomegalovirus (CMV) (such as the CMV promoter/enhancer), simian virus 40 (SV40) (such as the SV40 promoter/enhancer), from adenovirus (e.g., the adenovirus major late promoter (AdMLP)) and from polyoma. The expression of the antibodies can take place in a constitutive or regulated manner (e.g., induced by addition or removal of small molecule inducers such as tetracycline in combination with the Tet system).

For further description of viral regulatory elements and sequences thereof, reference is made, for example, to U.S. Pat. No. 5,168,062 by Stinski, U.S. Pat. No. 4,510,245 by Bell et al. and U.S. Pat. No. 4,968,615 by Schaffner et al. The recombinant expression vectors can likewise include a replication origin and selectable markers (see, for example, U.S. Pat. Nos. 4,399,216, 4,634,665 and 5,179,017). Suitable selectable markers include genes that confer resistance to substances such as G418, puromycin, hygromycin, blasticidin, zeocin/bleomycin, or methotrexate, or selectable markers that lead to auxotrophy of a host cell, such as glutamine synthetase (Bebbington et al., Biotechnology (N Y). 1992 February; 10(2):169-75), when the vector was inserted into the cell. For example, the dihydrofolate reductase (DHFR) gene imparts resistance to methotrexate, the neo gene imparts resistance to G418, the bsd gene from Aspergillus terreus imparts resistance to blasticidin, puromycin N-acetyl-transferase imparts resistance to puromycin, the Sh ble gene product imparts resistance to zeocin, and resistance to hygromycin is imparted by the E. coli hygromycin resistance gene (hyg or hph). Selectable markers such as DHFR or glutamine synthetase are also helpful for amplification techniques in connection with MTX and MSX. The transfection of an expression vector into a host cell can be done with the aid of standard techniques, using among others electroporation, nucleofection, calcium-phosphate-precipitation, lipofection, polycation-based transfection such as polyethyleneimine (PED-based transfection and DEAE-dextran transfection.

Suitable mammalian host cells for the expression of antibodies, antigen-binding fragments thereof, or variants thereof comprise Chinese hamster ovary (CHO) cells, such as CHO-K1, CHO—S, CHO-KISV [including DHFR-CHO cells, described in Urlaub and Chasin, (1980) Proc. Natl. Acad. Sci. USA 77:4216-4220 and Urlaub et al., Cell. 1983 June; 33(2):405-12, used with a DHFR-selectable marker, as described in R. J. Kaufman and P. A. Sharp (1982) Mol. Biol. 159:601-621, as well as other knockout cells, as listed in Fan et al., Biotechnol Bioeng. 2012 April; 109(4):1007-15), NSO myeloma cells, COS cells, HEK293 cells, HKB11 cells, BHK21 cells, CAP cells, EB66 cells, and SP2 cells.

The expression of antibodies, antigen-binding fragments thereof, or variants thereof can also take place in a transient or semi-stable manner in expression systems such as HEK293, HEK293T, HEK293-EBNA, HEK293E, HEK293-6E, HEK293-Freestyle, HKB11, Expi293F, 293EBNALT75, CHO Freestyle, CHO-S, CHO-K1, CHO-KISV, CHOEBNALT85, CHOS-XE, CHO-3E7 or CAP-T cells (for example as in Durocher et al., Nucleic Acids Res. 2002 Jan. 15; 30(2):E9)

In some embodiments the expression vector is constructed in that the protein to be expressed is secreted into the cell culture medium in which the host cells are growing. The antibody, the antigen-binding fragments thereof, or the variants thereof can be obtained from the cell culture medium with the aid of protein purification methods known to the person skilled in the art.

### Purification

The antibody, the antigen-binding fragments thereof, or the variants thereof can be obtained and purified from recombinant cell cultures using well known methods, comprising for example ammonium sulfate or ethanol precipitation, acid extraction, protein A chromatography, protein G chromatography, anion or cation exchange chromatography, phosphocellulose chromatography, hydrophobic interaction chromatography (HIC), affinity chromatography, hydroxyapatite chromatography and lectin chromatography. High performance liquid chromatography (HPLC) can also be used for purification. See, for example, Colligan, Current Protocols in Immunology, or Current Protocols in Protein Science, John Wiley & Sons, NY, N.Y., (1997-2001), e.g., Chapters 1, 4, 6, 8, 9, 10.

Antibodies of the present invention or antigen-binding fragments thereof, or the variants thereof comprise naturally purified products, products from chemical synthesis methods and products prepared using recombinant techniques in prokaryotic or eukaryotic host cells. Eukaryotic hosts comprise, for example, yeast cells, higher plant cells, insect cells and mammalian cells. Depending on the host cell selected for the recombinant expression, the protein expressed may exist in glycosylated or non-glycosylated form.

In a preferred embodiment the antibody is purified (1) to the extent of more than 95% by weight, measured for example with the Lowry method, with UV-Vis spectroscopy or with SDS capillary gel electrophoresis (for example with a Caliper LabChip GXII, GX 90 or Biorad Bioanalyzer instrument), and in more preferred embodiments more than 99% by weight, (2) to a degree suitable for determination of at least 15 residues of the N-terminal or internal amino acid sequence, or (3) to homogeneity determined by SDS-PAGE under reducing or non-reducing conditions using Coomassie blue or preferably silver staining.

Usually, an isolated antibody with is obtained with the aid of at least one protein purification step.

### Anti-CXCR5 Antibodies

According to the invention, anti-CXCR5 antibodies can be used.

The term “anti-CXCR5 antibody” or “an antibody that binds specifically to CXCR5” relates to an antibody that binds the cancer target molecule CXCR5 (NCBI reference sequence: NP_001707.1; SEQ ID NO 81), preferably with an affinity sufficient for a diagnostic and/or therapeutic application. In certain embodiments, the antibody CXCR5 binds with a dissociation constant (K_{D}) of ≤100 nM, ≤10 nM, ≤1 nM,

An example of an antibody- and antigen-binding fragment binding to human CXCR5 are known to the person skilled in the art as, for example, the rat antibody clone RF8B2 (ACC2153) or the human antibody 40C01 as described in WO2014/177652.

Particularly preferred in the context of this invention are the anti-CXCR5 antibodies TPP-14511, TPP-14509, TPP-14499, TPP-14505, TPP-14514 and TPP-14495. Precursors (e.g., TPP-10063) of the antibodies mentioned were selected by selection on peptides and cells using phage display technology and their properties subsequently optimized using protein engineering.

### Preferred Antibodies and Antigen-Binding Antibody Fragments for Binder/Active Agent Conjugates According to the Invention

In this application, the following preferred antibodies are used in the binder/active agent conjugates, as shown in the following table: TPP-14511, TPP-14509, TPP-14499, TPP-14505, TPP-14514 and TPP-14495.

TABLE
Protein sequences of the antibodies:

| | SEQ | SEQ ID | SEQ ID | SEQ ID | SEQ ID | SEQ ID | SEQ ID | SEQ ID | SEQ ID | SEQ ID |
|---|---|---|---|---|---|---|---|---|---|---|
| Antibodies | ID NO.: | NO.: | NO.: | NO. | NO.: | NO.: | NO.: | NO.: | NO.: IgG | NO.: IgG |
| TPP-XXX | VH | H-CDR1 | H-CDR2 | H-CDR3 | VL | l-CDR1 | L-CDR2 | L-CDR3 | heavy Chain | Light Chain |
| TPP-14495 | 1 | 2 | 3 | 4 | 5 | 6 | 7 | 8 | 9 | 10 |
| TPP-14499 | 11 | 12 | 13 | 14 | 15 | 16 | 17 | 18 | 19 | 20 |
| TPP-14505 | 21 | 22 | 23 | 24 | 25 | 26 | 27 | 28 | 29 | 30 |
| TPP-14509 | 31 | 32 | 33 | 34 | 35 | 36 | 37 | 38 | 39 | 40 |
| TPP-14511 | 41 | 42 | 43 | 44 | 45 | 46 | 47 | 48 | 49 | 50 |
| TPP-14514 | 51 | 52 | 53 | 54 | 55 | 56 | 57 | 58 | 59 | 60 |
| TPP-10063 | 61 | 62 | 63 | 64 | 65 | 66 | 67 | 68 | 69 | 70 |
| 40C01 | 71 | 72 | 73 | 74 | 75 | 76 | 77 | 78 | 79 | 80 |

TPP-14511, TPP-14509, TPP-14499, TPP-14505, TPP-14514, TPP-14495, TPP-10063 and 40C01 are antibodies comprising one or more of the CDR sequences shown in the above table (H-CDR1, H-CDR2, H-CDR3, L-CDR1, L-CDR2, L-CDR3) of the variable region of the heavy chain (VH) or of the variable region of the light chain (VL). Preferably the antibodies comprise the specified variable region of the heavy chain (VH) and/or the variable region of the light chain (VL). Preferably the antibodies comprise the specified region of the heavy chain (IgG heavy chain) and/or the specified region of the light chain (IgG light chain).

TPP-14495 is an anti-CXCR5 antibody comprising a variable region of the heavy chain (VH) comprising the variable CDR1 sequence of the heavy chain (H-CDR1), as shown by SEQ ID NO: 2, the variable CDR2 sequence of the heavy chain (H-CDR2), as shown by SEQ ID NO: 3 and the variable CDR3 sequence of the heavy chain (H-CDR3), as shown by SEQ ID NO: 4, as well as a variable region of the light chain (VL) comprising the variable CDR1 sequence of the light chain (L-CDR1), as shown by SEQ ID NO: 6, the variable CDR2 sequence of the light chain (L-CDR2), as shown by SEQ ID NO: 7 and the variable CDR3 sequence of the light chain (L-CDR3), as shown by SEQ ID NO: 8.

TPP-14499 is an anti-CXCR5 antibody comprising a variable region of the heavy chain (VH) comprising the variable CDR1 sequence of the heavy chain (H-CDR1), as shown by SEQ ID NO: 12, the variable CDR2 sequence of the heavy chain (H-CDR2), as shown by SEQ ID NO: 13 and the variable CDR3 sequence of the heavy chain (H-CDR3), as shown by SEQ ID NO: 14, as well as a variable region of the light chain (VL) comprising the variable CDR1 sequence of the light chain (L-CDR1), as shown by SEQ ID NO: 16, the variable CDR2 sequence of the light chain (L-CDR2), as shown by SEQ ID NO: 17 and the variable CDR3 sequence of the light chain (L-CDR3), as shown by SEQ ID NO: 18.

TPP-14505 is an anti-CXCR5 antibody comprising a variable region of the heavy chain (VH) comprising the variable CDR1 sequence of the heavy chain (H-CDR1), as shown by SEQ ID NO: 22, the variable CDR2 sequence of the heavy chain (H-CDR2), as shown by SEQ ID NO: 23 and the variable CDR3 sequence of the heavy chain (H-CDR3), as shown by SEQ ID NO: 24, as well as a variable region of the light chain (VL) comprising the variable CDR1 sequence of the light chain (L-CDR1), as shown by SEQ ID NO: 26, the variable CDR2 sequence of the light chain (L-CDR2), as shown by SEQ ID NO: 27 and the variable CDR3 sequence of the light chain (L-CDR3), as shown by SEQ ID NO: 28.

TPP-14509 is an anti-CXCR5 antibody comprising a variable region of the heavy chain (VH) comprising the variable CDR1 sequence of the heavy chain (H-CDR1), as shown by SEQ ID NO: 32, the variable CDR2 sequence of the heavy chain (H-CDR2), as shown by SEQ ID NO: 33 and the variable CDR3 sequence of the heavy chain (H-CDR3), as shown by SEQ ID NO: 34, as well as a variable region of the light chain (VL) comprising the variable CDR1 sequence of the light chain (L-CDR1), as shown by SEQ ID NO: 36, the variable CDR2 sequence of the light chain (L-CDR2), as shown by SEQ ID NO: 37 and the variable CDR3 sequence of the light chain (L-CDR3), as shown by SEQ ID NO: 38.

TPP-14511 is an anti-CXCR5 antibody comprising a variable region of the heavy chain (VH) comprising the variable CDR1 sequence of the heavy chain (H-CDR1), as shown by SEQ ID NO: 42, the variable CDR2 sequence of the heavy chain (H-CDR2), as shown by SEQ ID NO: 43 and the variable CDR3 sequence of the heavy chain (H-CDR3), as shown by SEQ ID NO: 44, as well as a variable region of the light chain (VL) comprising the variable CDR1 sequence of the light chain (L-CDR1), as shown by SEQ ID NO: 46, the variable CDR2 sequence of the light chain (L-CDR2), as shown by SEQ ID NO:47 and the variable CDR3 sequence of the light chain (L-CDR3), as shown by SEQ ID NO: 48.

TPP-14514 is an anti-CXCR5 antibody comprising a variable region of the heavy chain (VH) comprising the variable CDR1 sequence of the heavy chain (H-CDR1), as shown by SEQ ID NO: 52, the variable CDR2 sequence of the heavy chain (H-CDR2), as shown by SEQ ID NO: 53 and the variable CDR3 sequence of the heavy chain (H-CDR3), as shown by SEQ ID NO: 54, as well as a variable region of the light chain (VL) comprising the variable CDR1 sequence of the light chain (L-CDR1), as shown by SEQ ID NO: 56, the variable CDR2 sequence of the light chain (L-CDR2), as shown by SEQ ID NO: 57 and the variable CDR3 sequence of the light chain (L-CDR3), as shown by SEQ ID NO: 58.

TPP-10063 is an anti-CXCR5 antibody comprising a variable region of the heavy chain (VH) comprising the variable CDR1 sequence of the heavy chain (H-CDR1), as shown by SEQ ID NO: 62, the variable CDR2 sequence of the heavy chain (H-CDR2), as shown by SEQ ID NO: 63 and the variable CDR3 sequence of the heavy chain (H-CDR3), as shown by SEQ ID NO: 64, as well as a variable region of the light chain (VL) comprising the variable CDR1 sequence of the light chain (L-CDR1), as shown by SEQ ID NO: 66, the variable CDR2 sequence of the light chain (L-CDR2), as shown by SEQ ID NO: 67 and the variable CDR3 sequence of the light chain (L-CDR3), as shown by SEQ ID NO: 68.

TPP-14495 is an anti-CXCR5 antibody preferably comprising a variable region of the heavy chain (VH) corresponding to SEQ ID NO: 1 as well as a variable region of the light chain (VL) corresponding to SEQ ID NO: 5.

TPP-14499 is an anti-CXCR5 antibody preferably comprising a variable region of the heavy chain (VH) corresponding to SEQ ID NO: 11 as well as a variable region of the light chain (VL) corresponding to SEQ ID NO: 15.

TPP-14505 is an anti-CXCR5 antibody preferably comprising a variable region of the heavy chain (VH) corresponding to SEQ ID NO: 21 as well as a variable region of the light chain (VL) corresponding to SEQ ID NO: 25.

TPP-14509 is an anti-CXCR5 antibody preferably comprising a variable region of the heavy chain (VH) corresponding to SEQ ID NO: 31 as well as a variable region of the light chain (VL) corresponding to SEQ ID NO: 35.

TPP-14511 is an anti-CXCR5 antibody preferably comprising a variable region of the heavy chain (VH) corresponding to SEQ ID NO: 41 as well as a variable region of the light chain (VL) corresponding to SEQ ID NO: 45.

TPP-14514 is an anti-CXCR5 antibody preferably comprising a variable region of the heavy chain (VH) corresponding to SEQ ID NO: 51 as well as a variable region of the light chain (VL) corresponding to SEQ ID NO: 55.

TPP-10063 is an anti-CXCR5 antibody preferably comprising a variable region of the heavy chain (VH) corresponding to SEQ ID NO: 61 as well as a variable region of the light chain (VL) corresponding to SEQ ID NO: 65.

TPP-14495 is an anti-CXCR5 antibody preferably comprising a region of the heavy chain corresponding to SEQ ID NO: 9 as well as a region of the light chain corresponding to SEQ ID NO: 10.

TPP-14499 is an anti-CXCR5 antibody preferably comprising a region of the heavy chain corresponding to SEQ ID NO: 19 as well as a region of the light chain corresponding to SEQ ID NO: 20.

TPP-14505 is an anti-CXCR5 antibody preferably comprising a region of the heavy chain corresponding to SEQ ID NO: 29 as well as a region of the light chain corresponding to SEQ ID NO: 30.

TPP-14509 is an anti-CXCR5 antibody preferably comprising a region of the heavy chain corresponding to SEQ ID NO: 39 as well as a region of the light chain corresponding to SEQ ID NO: 40.

TPP-14511 is an anti-CXCR5 antibody preferably comprising a region of the heavy chain corresponding to SEQ ID NO: 49 as well as a region of the light chain corresponding to SEQ ID NO: 50.

TPP-14514 is an anti-CXCR5 antibody preferably comprising a region of the heavy chain corresponding to SEQ ID NO: 59 as well as a region of the light chain corresponding to SEQ ID NO: 60.

TPP-10063 is an anti-CXCR5 antibody preferably comprising a region of the heavy chain corresponding to SEQ ID NO: 69 as well as a region of the light chain corresponding to SEQ ID NO: 70.

40C01 is an anti-CXCR5 antibody as described in WO2014/177652 and represented here by sequences specified in the above table (SEQ ID NO: 71-80).

### Isotopes, Salts, Solvates, Isotopic Variants

The present invention also comprises all suitable isotopic variants of the compounds according to the invention. Here, an isotopic variant of a compound according to the invention is defined as a compound in which at least one atom in the compound according to the invention has been exchanged for another atom of the same atomic number, but with a different atomic mass from the atomic mass usually or predominantly occurring in nature. Examples of isotopes that can be incorporated in a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷0, ¹⁸0, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Certain isotopic variants of a compound according to the invention, particularly those in which one or more radioactive isotopes are incorporated, can be beneficial, for example for investigating the mechanism of action of the active agent or the distribution of the active agent in the body because of the relatively easy preparation and detection, are especially compound labeled with ³H or ¹⁴C isotopes. In addition, the incorporation of isotopes, for example of deuterium, may give rise to certain therapeutic benefits as a result of greater metabolic stability of the compound, for example prolongation of the half-life in the body or reduction of the required effective dose; such modifications of the compounds according to the invention can therefore optionally also represent a preferred embodiment of the present invention. Isotopic variants of the compounds according to the invention can be prepared according to the methods known to the person skilled in the art and the descriptions in the exemplary embodiments by using corresponding isotopic modifications of the respective reagents and/or starting compounds.

Preferred salts in the context of the present invention are physiologically acceptable salts of the compounds according to the invention. Also included are salts which themselves are unsuitable for pharmaceutical applications, but which can be used, for example, for isolation or purification of the compounds according to the invention.

Physiologically acceptable salts of the compounds according to the invention comprise acid addition salts of mineral acids, carboxylic acids and sulfonic acids, e.g., salts of hydrochloric acid, hydrobromic acid, sulfuric acid, phosphoric acid, methanesulfonic acid, ethanesulfonic acid, benzenesulfonic acid, toluenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also comprise salts of common bases, for example and preferably alkali metal salts (e.g., sodium and potassium salts), alkaline earth salts (e.g., calcium and magnesium salts), alkali metal salts (e.g., sodium and potassium salts), alkaline earth salts (e.g., calcium and magnesium salts) and ammonium salts derived from ammonia or organic amines with 1 to 16 C atoms, for example preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylpiperidine, N-methylmorpholine, arginine, lysine and 1,2-ethylenediamine.

Solvates used in the context of the invention are those forms of the compounds according to the invention that form a complex in the solid or liquid state by coordination with solvent molecules. Hydrates are a special form of solvates in which the coordination takes place with water. Preferred solvates in the context of the present invention are hydrates.

### Therapeutic Use

The hyperproliferative diseases in the treatment of which the compounds according to the invention can be used include in particular the group of cancers and tumor diseases. In the context of the present invention, these are understood to mean particularly the following diseases, but without being limited to them: breast carcinomas and breast tumors (mammary carcinomas including ductal and lobular forms, also in situ), tumors of the respiratory tract (small-cell and non-small cell carcinoma, bronchial carcinoma), brain tumors (e.g., of the brain stem and the hypothalamus, astrocytoma, ependymoma, glioblastoma, glioma, medulloblastoma, meningioma as well as neuroectodermal and pineal tumors), tumors of the digestive organs (carcinomas of the esophagus, stomach, gall bladder, small intestine, large intestine, rectal and anal carcinomas), liver tumors (including hepatocellular carcinoma, cholangiocarcinoma and mixed hepatocellular cholangiocarcinoma), head and neck tumors (carcinomas of the larynx, hypopharynx, nasopharynx, oropharynx, lips and oral cavities, oral melanomas), skin tumors (basaliomas, spinaliomas, squamous cell carcinomas, Kaposi sarcoma, malignant melanoma, non-melanomatous skin cancer, Merkel cell skin cancer, mast cell tumors), tumors of the supporting and connective tissue (among others soft tissue sarcomas, osteosarcomas, malignant fibrous histiocytomas, chondrosarcomas, fibrosarcomas, hemangiosarcomas, leiomyosarcomas, liposarcomas, lymphosarcomas and rhabdomyosarcomas), tumors of the eyes (including intraocular melanoma and retinoblastoma), tumors of the endocrine and exocrine glands (e.g., of the thyroid and parathyroid glands, pancreatic and salivary gland carcinomas, adenocarcinomas), urinary trac tumors (tumors of the bladder, penis, renal pelvis and ureter) and tumors of the reproductive organs (carcinomas of the endometrium, cervix, ovaries, vagina, vulva and uterus in women as well as carcinomas of the prostate and testicles in men). Also included are proliferative diseases of the blood, the lymphatic system and the bone marrow, in solid form and as circulating cells, such as leukemias, lymphomas and myeloproliferative diseases, e.g., acute myeloid, acute lymphoblastic, chronic-lymphocytic, chronic-myelogenous and hairy cell leukemia, as well as AIDS-related lymphomas, Hodgkin's lymphomas, non-Hodgkin's-lymphomas, cutaneous T-cell lymphomas, Burkitt lymphomas and central nervous system lymphomas.

These diseases, well characterized in humans, can also occur with comparable etiology in other mammals, and in these also can be treated with the compounds of the present invention. The binder- or antibody-drug conjugates (ADCs) directed against CXCR5 described here can preferably be used to treat CXCR5-expressing disorders, such as CXCR5-expressing cancers. Typically, such cancer cells exhibit measurable amounts of CXCR5 measured at the protein level (e.g., by immunoassay) or RNA level. Some of these cancer tissues exhibit an elevated level of CXCR5 compared with noncancerous tissue of the same type, preferably measured in the same patient. Optionally the content of CXCR5 is measured before the cancer treatment with an antibody-drug conjugate (ADC) is initiated (patient stratification). The CXCR5-directed binder-drug conjugates (ADCs) can preferably be used to treat CXCR5-expressing disorders, such as CXCR5-expressing cancers such as tumors of the hematopoietic and lymphatic tissue or hematopoietic and lymphatic malignant tumors. Examples of cancers associated with CXCR5 expression include lymphatic diseases such as Burkitt lymphoma, follicular lymphoma, chronic lymphatic leukemia (CLL), mantle cell lymphoma (MCL), diffuse large B-cell lymphoma (DLBCL) and Hodgkin's lymphoma. In addition, increased expression of CXCR5 can also be found in solid tumors such as tumors of the breast, prostate, stomach and colon.

Methods of the invention described comprise the treatment of patients with a CXCR5 expressing cancer, wherein the method comprises the administration of an antibody-drug conjugate (ADC) according to the invention.

The treatment of the aforementioned cancers with the compounds according to the invention comprises both treatment of the solid tumor and treatment of metastatic or circulating forms thereof.

The term “treatment” or “treating” is used in the conventional sense in this invention and means attending to, nursing and caring for a patient with the goal of combating, reducing, ameliorating or alleviating a disease or health abnormality and improving the living conditions impaired by this disease, for example in the case of cancer.

Thus an additional subject of the present invention is the use of the compounds according to the invention for the treatment and/or prevention of diseases, particularly the aforementioned diseases.

An additional subject of the present invention is the use of the for preparing a medication for the treatment and/or prevention of diseases, particularly the aforementioned diseases.

An additional subject of the present invention is the use of the compounds according to the invention in a method for treatment and/or prevention of diseases, particularly the aforementioned diseases.

An additional subject of the present invention is a method for treatment and/or prevention of diseases, particularly the aforementioned diseases, using an effective quantity of at least one of the compounds invention.

The compounds according to the invention can be used alone or if necessary in combination with one or more other pharmacologically active agents, as long as this combination does not lead to undesirable and unacceptable side effects. An additional subject of the present invention is therefore the provision of medications containing at least one of the compounds according to the invention and one or more additional active agents, particularly for the treatment and/or prevention of the aforementioned diseases.

For example, the compounds of the present invention can be combined with known antihyperproliferative, cytostatic, cytotoxic or immunotherapeutic substances for treatment of cancers. Examples of suitable combination active agents include: 131 I-chTNT, abarelix, abemaciclib, abiraterone, acalabrutinib, aclarubicin, adalimumab, ado-trastuzumab emtansin, afatinib, aflibercept, aldesleukin, alectinib, alemtuzumab, alendronic acid, alitretinoin, altretamine, amifostine, aminoglutethimide, hexyl-5-aminolevulinate, amrubicin, amsacrin, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagen ciloleucel, axitinib, azacitidine, basiliximab, belotecan, bendamustine, besilesomab, belinostat, bevacizumab, bexaroten, bicalutamide, bisantren, bleomycin, blinatumomab, bortezomib, buserelin, bosutinib, brentuximab vedotin, brigatinib, busulfan, cabazitaxel, cabozantinib, calcitonin, calcium folinate, calcium levofolinate, capecitabine, capromab, carbamazepine, carboplatin, carboquon, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabin, cobimetinib, copanlisib, crisantaspase, crizotinib, cyclophosphamide, ciproterone, cytarabine, dacarbazine, dactinomycin, daratumumab, darbepoetin alpha, dabrafenib, darolutamide, dasatinib, daunorubicin, decitabine, degarelix, denileukin-diftitox, denosumab, depreotide, deslorelin, dexrazoxane, dibrospidium chloride, dianhydrogalactitol, dinutuximab, diclofenac, docetaxel, dolasetron, doxifluridine, doxorubicin, doxorubicin+estrone, dronabinol, durvalumab, eculizumab, edrecolomab, elliptinium acetate, endostatin, enocitabine, enzalutamide, epacadostat, epirubicin, epitiostanol, epoetin-alfa, epoetin-beta, epoetin-zeta, eptaplatin, eribulin, erlotinib, esomeprazole, estradiol, estramustin, etoposide, ethinyl estradiol, everolimus, exemestane, fadrozole, fentanyl, filgrastim, fluoxymesterone, floxuridine, fludarabine, fluoruracil, flutamide, folinic acid, formestan, fosaprepitant, fotemustine, fulvestrant, gadobutrol, gadoteridol, gadoteric acid-meglumine salt, gadoversetamide, gadoxetic acid disodium salt (gd-eob-dtpa disodium salt), gallium nitrate, ganirelix, gefitinib, gemcitabine, gemtuzumab, glucarpidase, glutoxime, goserelin, granisetron, granulocyte colony stimulating factor (g-csf), granulocyte-macrophage colony stimulating factor (gm-csf), histamine dihydrochloride, histrelin, hydroxycarbamide, I-125-seeds, lansoprazole, ibandronic acid, ibritumomab-tiuxetan, ibrutinib, idarubicin, ifosfamide, imatinib, imiquimod, improsulfan, indisetron, incadronic acid, ingenolmebutate, inotuzumab ozogamicin, interferon-alfa, interferon-beta, interferon-gamma, iobitridol, iobenguan (1231), iomeprol, ipilimumab, irinotecan, itraconazole, ixabepilone, ixazomib, lanreotide, lansoprazole, lansoprazol, lapatinib, lasocholine, lenalidomide, lenvatinib, lenograstim, lentinan, letrozole, leuprorelin, levamisole, levonorgestrel, levothyroxine sodium, lipegfilgrastim, lisuride, lobaplatin, lomustine, lonidamine, lutetium Lu 177 dotatate, masoprocol, medroxyprogesterone, megestrol, melarsoprol, melphalan, mepitiostan, mercaptopurine, mesna, methadone, methotrexate, methoxsalen, methylaminolevulinate, methylprednisolone, methyltestosterone, metirosin, midostaurin, mifamurtide, miltefosin, miriplatin, mitobronitol, mitoguazone, mitolactol, mitomycin, mitotan, mitoxantrone, mogamulizumab, molgramostim, mopidamol, morphine hydrochloride, morphine sulfaet, mvasi, nabilone, nabiximols, nafarelin, naloxone+pentazocine, naltrexone, nartograstim, necitumumab, nedaplatin, nelarabin, neratinib, neridronic acid, netupitant/palonosetron, nivolumab, nivolumab pentetreotid, nilotinib, nilutamide, nimorazol, nimotuzumab, nimustine, nintedanib, niraparib, nitracrin, nivolumab, obinutuzumab, octreotide, ofatumumab, olaparib, olaratumab, omacetaxin-mepesuccinate, omeprazole, ondansetron, oprelvekin, orgotein, orilotimod, osimertinib, oxaliplatin, oxycodone, oxymetholone, ozogamicin, p53-gentherapie, paclitaxel, palbociclib, palifermin, palladium-103 seeds, palonosetron, pamidronic acid, panitumumab, panobinostat, pantoprazole, pazopanib, pegaspargase, peg-epoetin beta (methoxy peg-epoetin beta), pembrolizumab, pegfilgrastim, peg-interferon-alfa-2b, pemetrexed, pentazocine, pentostatin, peplomycin, perflubutane, perfosfamide, pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-k, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifen, raltitrexed, ramosetron, ramucirumab, ranimustine, rasburicase, razoxan, refametinib, regorafenib, ribociclib, risedronic acid, rhenium-186 etidronat, rituximab, rogaratinib, rolapitant, romidepsin, romiplostim, romurtid, roniciclib, rucaparib, samarium (153sm) lexidronam, sargramostim, sarilumab, satumomab, secretin, siltuximab, sipuleucel-t, sodium glycididazole, sonidegib, sorafenib, stanozolol, sizofiran, obuzoxan, streptozocin, sunitinib, talaporfin, talimogen laherparepvec, tamibaroten, tamoxifen, tapentadole, tasonermin, teceleukin, technetium (99mtc) nofetumomab merpentan, 99mtc-HYNIC-[tyr3]-octreotide, tegafur, tegafur+gimeracil+oteracil, temoporfin, temozolomide, temsirolimus, teniposid, testosterone, tetrofosmin, thalidomide, thiotepa, thymalfasin, thyrotropin alfa, tioguanine, tisagenlecleucel, tocilizumab, topotecan, toremifen, tositumomab, trabectedin, trametinib, tramadol, trastuzumab, trastuzumab emtansin, treosulfan, trofosfamide, thrombopoietin, tryptophan, ubenimex, valrubicin, vandetanib, vapreotidw, valatinib, vemurafenib, vinblastine, vincristine, vindesine, vinflunin, vinorelbinw, vismodegib, vorinostat, vorozolw, yttrium-90-glass microbeads, zinostatin, zinostatin-stimalamer, zoledronic acid, zorubicin.

In addition, the compounds of the present invention can be combined, for example, with binders (e.g., antibodies) which may, for example, bind to the following targets can: OX-40, CD137/4-1BB, DR3, IDO1/IDO2, LAG-3, CD40.

Since a non-cell-permeable toxophore metabolite of a binder-drug conjugate (ADC) should not have any damaging effect on the cells of the adaptive immune system, the combination of a binder-drug conjugate (ADC) according to the invention with a cancer immunotherapy for use in the treatment of cancer or tumors is an additional subject of this invention. The intrinsic mechanism of action of cytotoxic binder/active agent conjugates comprises the direct triggering of cell death of the tumor cells and thus the release of tumor antigens that can stimulate an immune response. There are also indications that the KSP inhibitor-toxophore class induces markers of so-called immunogenic cell death [ICD] in vitro. Thus the combination of the binder-drug conjugates (ADCs) of the present invention with one or more therapeutic approaches of cancer immunotherapy or with one or more active agents, preferably antibodies, directed against a molecular target from cancer immunotherapy, represents a preferred method for treating cancer or tumors. i) Examples of therapeutic approaches for cancer immunotherapy comprise immuno-modulatory monoclonal antibodies and low-molecular-weight substances directed against targets from cancer immunotherapy, vaccines, CAR T cells, bispecific T cell-recruiting antibodies, oncolytic viruses, cell-based vaccination approaches. ii) Examples of selected targets from cancer immunotherapy suitable for immunomodulatory monoclonal antibodies comprise CTLA-4, PD-1/PDL-1, OX-40, CD137, DR3, IDOI, IDO2, TDO2, LAG-3, TIM-3, CD40, ICOS/ICOSL, TIGIT, GITR/GITRL, VISTA, CD70, CD27, HVEM/BTLA, CEACAMI, CEACAM6, ILDR2, CD73, CD47, B7H3 and TLRs. Therefore the combination of a binder-drug conjugate (ADC) according to the invention with a cancer immunotherapy could, on one hand, make tumors with weak immunogenic properties more immunogenic and enhance the effectiveness of cancer immunotherapy, and furthermore unfold long-acting therapeutic action.

In addition, the compounds according to the invention can also be used in combination with radiation therapy and/or a surgical procedure.

In general, the following goals can be pursued with the combination of compounds of the present invention with other agents of cytostatic, cytotoxic or immunotherapeutic activity:
improved efficacy in slowing the growth of a tumor, by reducing its size or even eliminating it completely in contrast to treatment with a single active agent,
the possibility of using the selected chemotherapeutic agents at a lower dosage than in the case of monotherapy;
the possibility of better tolerated therapy with fewer adverse effects compared with monotherapy;
the possibility of treating a broader spectrum of tumors;
attainment of a higher rate of response to therapy
longer survival time for patients compared with current standard therapy.

In addition, the compounds according to the invention can also be used in combination with radiation therapy and/or surgery.

Additional subjects of the present invention are medications containing at least one compound according to the invention together with one or more inert, nontoxic, pharmaceutically acceptable excipients and the use thereof for the aforementioned purposes.

The compounds according to the invention can act systemically and/or locally. They can be applied appropriately for this purpose, for example parenterally, possibly by inhalation or as an implant or stent.

Compounds according to the invention in suitable administration forms can be administered for these routes of administration.

Parenteral administration can be conducted while circumventing an absorption step (e.g., intravenous, intra-arterial, intracardiac, intraspinal or intralumbar) or including resorption (e.g., intramuscular, subcutaneous, intracutaneous, percutaneous or intraperitoneal). Suitable administration forms for parenteral administration include injections and infusion preparations in the form of solutions, suspensions, emulsions or lyophilizates. Parenteral administration, particularly intravenous administration, is preferred.

In general, it has proven advantageous in parenteral administration to apply quantities from about 0.1 to 20 mg/kg, preferably about 0.3 to 10 mg/kg body weight to achieve more effective results.

Nevertheless, it may sometimes be necessary to deviate from the quantities mentioned, specifically depending on body weight, route of administration, individual response to the active agent, nature of the preparation and time or interval at which the administration is given. For example, in some cases it may be sufficient to manage with less than the aforementioned minimum quantity, whereas in other cases the upper limit mention must be exceeded. When larger amounts are to be administered, it may be advantageous to distribute them in several individual doses over the day.

### EXAMPLES

The following examples will explain the invention. The invention is not limited to these examples.

Unless otherwise specified, the percentages given in the following tests and examples are percent by weight. All solvent ratios, dilution ratios and concentration data for liquid-liquid solutions are by volume.

### Synthesis Pathways:

The diagrams that follow represent examples for the exemplary embodiments.

Diagram 1: Synthesis of Lysine-Linked ADCs with Legumain-Cleavable Linkers

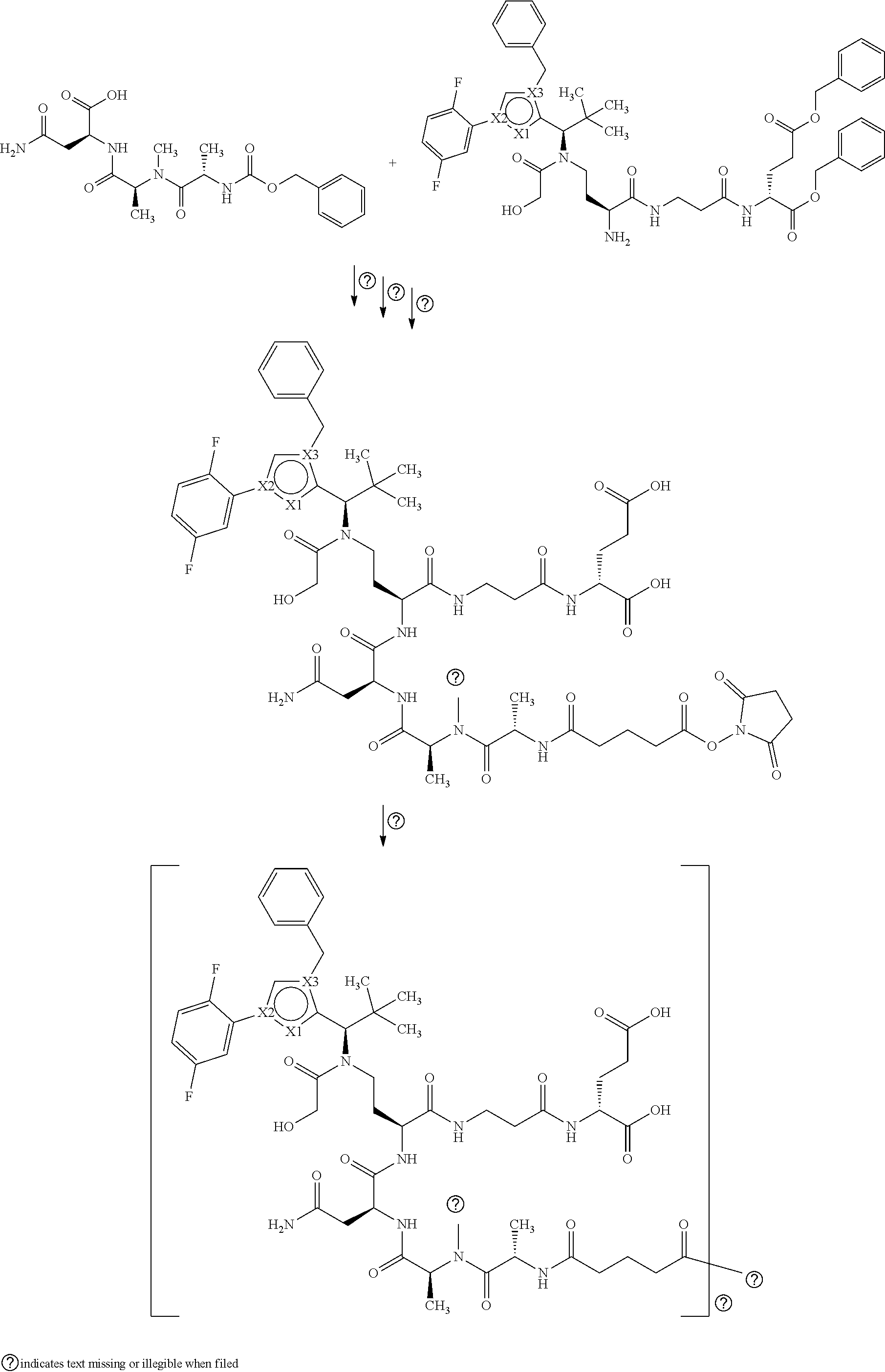

In the above reaction scheme, X₁, X₂, X₃, n and AK₂ have the meanings specified in formula (I).

a) HATU, DMF, N,N-diisopropylethylamine, RT; b) H₂, 10% Pd—C, methanol 1.5 h, RT; c) 1,1′-[(1,5-dioxopentane-1,5-diyl)bis(oxy)]dipyrrolidine-2,5-dione, N,N-diisopropyl-ethylamine, DMF, stir overnight at RT; d) AK₂ in PBS, under argon add 3-5 equiv. active ester dissolved in in DMSO, stir 60 min at RT under argon, again add 3-5 equiv. active ester dissolved in in DMSO, stir 60 min at RT under argon, then clean up over PD 10 columns (Sephadex® G-25, GE Healthcare) equilibrated with PBS buffer (pH 7.2) and followed by concentrating by ultracentrifugation, adjusting to the desired concentration with PBS buffer (pH 7.2)]. For in vivo batches, a sterile filtration may follow.

### A. Examples

### Abbreviations and Acronyms

ABCBl ATP-binding cassette sub-family B member 1 (synonymous for P-gp and MDRI) Absolute
abs. Acetyl
Ac ACN Acetonitrile
aq. Aqueous, aqueous solution
ATP Adenosine triphosphate
BCRP Breast cancer resistance protein, an efflux transporter
BEP 2-bromo-1-ethylpyridinium tetrafluoroborate
Boc tert.-Butoxycarbonyl
br. Broad (in NMR)
Bsp. Example
C Concentration
approx. circa, approximately
CI Chemical ionization (in MS)
DAR Drug-to-antibody ratio
D Doublet (in NMR)
D Day(s)
DC Thin-layer chromatography
DCI DCM Direct chemical ionization (in MS)
Dd Dichloromethane Doublet of doublets (in NMR)
DMAP 4-N,N-Dimethylaminopyridine
DME 1,2-Dimethoxyethane
DMEM Dulbecco's Modified Eagle Medium (standardized nutrient medium for cell culture)
DMF N,N-Dimethylformamide
DMSO Dimethyl sulfoxide
D/P Dye (fluorescent dye)/protein ratio
DPBS, D-PBS Dulbecco's phosphate-buffered saline-solution
DSMZ Deutsche Sammlung von Mikroorganismen and Zellkulturen [German Collection of Microorganisms and Cell Cultures]
PBS PBS=DPBS=D-PBS, pH 7.4, Sigma, No. D8537
   Composition:
   0.2 g KCl
   0.2 g KH₂PO₄ (anhyd.) 8.0 g NaCl
   1.15 g Na₂HPO₄ (anhyd.) fill with H₂O to make 1 L
Dt DTT Doublet of triplets (in NMR)
d. Th. DL-Dithiothreitol
EDC of theoretical (chemical yield) N-(3-Dimethylaminopropyl)-N-ethylcarbodiimide hydrochloride
EGFR Epidermal growth factor receptor
EI Electron impact ionization (in MS) Enzyme-linked
ELISA immunosorbent assay
eq. Equivalent(s)
ESI Electrospray Ionization (in MS)
ESI-Micro-ER MicroTofq (name of mass spectrometer with Tof=Time Of Flight and
Tofq q=Quadrupole)
FCS Fetal calf serum
Fmoc (9H-Fluoren-9-ylmethoxy)carbonyl
ges. Saturated
GTP Guanosine-5′-triphosphate
H Hour(s)
HATU O-(7-Azabenzotriazol-1-yl)-N,N,N′,N′-tetramethyluronium hexafluorophosphate
HBL-1 Human tumor cell line
HEPES 4-(2-Hydroxyethyl)piperazine-1-ethanesulfonic acid
HOAc Acetic acid
HOAt 1-Hydroxy-7-azabenzotriazole
HOBt 1-Hydroxy-IH-benzotriazole hydrate
HOSu N-Hydroxysuccinimide
HPLC High-pressure, high-performance liquid chromatography
IC₅₀ Half-maximal inhibition concentration
i.m. Intramuscular, administration into the muscle
i.v. Intravenous, administration into the vein
conc. Concentrated
LC-MS Liquid chromatography-coupled mass spectrometry
LLC-PKI cells Lewis lung carcinoma pork kidney cell line
L-MDR Human MDRI transfected LLC-PKI cells
M Multiplet (in NMR)
Me Methyl
MDR1 Multidrug resistance protein 1
MeCN Acetonitrile
Min Minute(s)
MS Mass spectrometry
MTT 3-(4,5-Dimethylthiazol-2-yl)-2,5-diphenyl-2H-tetrazolium bromide
NCI-H292 Human tumor cell line
NMM N-methylmorpholine
NMP N-methyl-2-pyrrolidinone
NMR Nuclear magnetic resonance spectrometry
NMRI Mouse strain from Naval Medical Research Institute (NMRI)
Nude mouse Nude mouse (experimental animal)
NSCLC Non-small-cell lung cancer (non-small-cell bronchial carcinoma)
Oci-Ly-1 PBS Human tumor cell line
Pd/C Phosphate-buffered salt solution
P-gp Palladium on active carbon
PNGaseF P-Glycoprotein, a transporter protein
quant. Enzyme for splitting off sugar
Quart Quantitative (yield)
Quint Quartet (in NMR)
Rec-1 Quintet (in NMR)
Rf Human tumor cell line
RT Retention index (in TLC)
Rt Room temperature
s Retention time (in HPLC)
s.c. Singlet (in NMR)
SCID Mouse Subcutaneous, administration under the skin
SU-DHL6 Experimental mouse with severe combined immunodeficiency
T Human tumor cell line
TBAF Triplet (in NMR)
TCEP TEMPO Tetra-n-butylammonium fluoride
Teoc Tris(2-carboxyethyl)phosphine (2,2,6,6-Tetramethyl-piperidin-1-yl)oxyl Trimethylsilylethoxycarbonyl
tert. Tertiary
TFA Trifluoroacetic acid
THF Tetrahydrofuran
T3P® 2,4,6-Tripropyl-1,3,5,2,4,6-trioxatriphosphinane-2,4,6-trioxide
UV Ultraviolet spectrometry
v/v Volume to volume ratio (of a solution)
Z Benzyloxycarbonyl

### HPLC and LC-MS Methods:

### Method 1 (LC-MS):

Instrument: Waters ACQUITY SQD UPLC System; column: Waters Acquity UPLC HSS T3 1.8μ 50×1 mm; Eluent A: 1 l water+0.25 mL 99% formic acid, Eluent B: 1 L acetonitrile+0.25 mL 99% formic acid; gradient: 0.0 min 90% A→1.2 min 5% A→2.0 min 5% A Oven: 50° C.; flow rate: 0.40 mL/min; UV detection: 208-400 nm.

### Method 6 (LC-MS):

Instrument: Micromass Quattro Premier with Waters UPLC Acquity; column: Thermo Hypersil GOLD 1.9μ 50×1 mm; Eluent A: 1 l water+0.5 mL 50% formic acid, Eluent B: 1 l acetonitrile+0.5 mL 50% formic acid; gradient: 0.0 min 97% A→0.5 min 97% A→3.2 min 5% A→4.0 min 5% A Oven: 50° C.; flow rate: 0.3 mL/min; UV detection: 210 nm.

### Method 7 (LC-MS):

Instrument: Agilent MS Quad 6150; HPLC: Agilent 1290; column: Waters Acquity UPLC HSS T3 1.8μ 50×2.1 mm; Eluent A: 1 l water+0.25 mL 99% formic acid, Eluent B: 1 l acetonitrile+0.25 mL 99% formic acid; gradient: 0.0 min 90% A→0.3 min 90% A→1.7 min 5% A→3.0 min 5% A Oven: 50° C.; flow rate: 1.20 mL/min; UV detection: 205-305 nm.

### Method 12 (LC-MS):

Instrument MS: Thermo Scientific FT-MS; instrument UHPLC+: Thermo Scientific UltiMate 3000; column: Waters, HSST3, 2.1×75 mm, C18 1.8 μm; Eluent A: 1 l water+0.01% formic acid; Eluent B: 1 l acetonitrile+0.01% formic acid; gradient: 0.0 min 10% B→2.5 min 95% B→3.5 min 95% B; Oven: 50° C.; flow rate: 0.90 ml/min; UV detection: 210 nm/Optimum Integration Path 210-300 nm.

All reactants or reagents whose preparation is not explicitly described in the following were purchased commercially from generally available sources. For all other reactants or reagents whose preparation is likewise not explicitly described in the following and which were not commercially available or were purchased from sources that are not generally accessible, references are given to the published literature in which their preparation is described.

### Starting Compounds and Intermediates

### Intermediate C52

### (1R)-1-[1-Benzyl-4-(2,5-difluorophenyl)-1H-pyrrol-2-yl]-2,2-dimethylpropan-1-amine

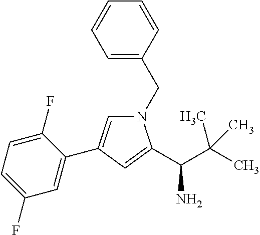 [SMILES: CC(C)(C)[C@@H](N)c1cc(-c2cc(F)ccc2F)cn1Cc1ccccc1]

10.00 g (49.01 mmol) of methyl-4-bromo-1H-pyrrole-2-carboxylate in 100.0 mL DMF were placed in a receptacle and 20.76 g (63.72 mmol) cesium carbonate and 9.22 g (53.91 mmol) benzyl bromide were added. The reaction mixture was stirred overnight at RT. The reaction mixture was partitioned between water and ethyl acetate and the aqueous phase extracted with ethyl acetate. The combined organic phases were dried over magnesium sulfate and the solvent evaporated to dryness under vacuum. The reaction was repeated with 90.0 g methyl-4-bromo-1H-pyrrole-2-carboxylate.

The combined two batches were cleaned up by preparative RP-HPLC (column: Daiso 300×100; 10μ, flow rate: 250 mL/min, MeCN/water). The solvents were evaporated under vacuum and the residue dried under high vacuum. The product was 125.15 g (87% of theoretical) of the compound methyl-1-benzyl-4-bromo-1H-pyrrole-2-carboxylate.

LC-MS (Method 1): Rₜ=1.18 min; MS (ESipos): m/z=295 [M+H)⁺.

Under argon, 4.80 g (16.32 mmol) methyl-1-benzyl-4-bromo-1H-pyrrole-2-carboxylate was placed in DMF, and 3.61 g (22.85 mmol) (2,5-difluorophenyl)boronic acid and 19.20 mL saturated sodium carbonate solution and 1.33 g (1.63 mmol) [1,1′-Bis-(diphenylphosphino)-ferrocene]-dichloropalladium(II):dichloromethane were added. The reaction mixture was stirred overnight at 85° C. The reaction mixture was filtered over Celite and the filter cake was washed with ethyl acetate. The organic phase was extracted with water and then washed with saturated NaCl solution. The organic phase was dried over magnesium sulfate and the solvent evaporated under vacuum. The residue was purified on silica gel (mobile phase: cyclohexane/ethyl acetate 100:3). The solvents were evaporated under vacuum and the residue dried under high vacuum. This gave 3.60 g (67% of theoretical) of the compound methyl-1-benzyl-4-(2,5-difluorophenyl)-1H-pyrrole-2-carboxylate.

LC-MS (Method 7): Rₜ=1.59 min; MS (ESipos): m/z=328 [M+H]⁺.

3.60 g (11.00 mmol) methyl-1-benzyl-4-(2,5-difluorophenyl)-1H-pyrrole-2-carboxylate were placed in 90.0 mL THF and treated at 0° C. with 1.04 g (27.50 mmol) lithium aluminum hydride (2.4 M in THF). The reaction mixture was stirred for 30 minutes at 0° C. Saturated potassium sodium tartrate solution was added at 0° C. and ethyl acetate was added to the reaction mixture. The organic phase was extracted three times with saturated potassium sodium tartrate solution. The organic phase was washed once with saturated NaCl solution and dried over magnesium sulfate. The was evaporated under vacuum and the residue dissolved in 30.0 mL dichloromethane. Then 3.38 g (32.99 mmol) manganese(IV)oxide were added and the mixture stirred for 48 h at RT. An additional 2.20 g (21.47 mmol) manganese(IV)oxide were added and stirred overnight at RT. The reaction mixture was filtered over Celite and the filter cake was washed with dichloromethane. The solvent was evaporated under vacuum and the residue, 2.80 g (1-benzyl-4-(2,5-difluorophenyl)-1H-pyrrole-2-carbaldehyde), was used in the next synthesis step without further purification.

LC-MS (Method 7): Rₜ=1.48 min; MS (ESipos): m/z=298 [M+H]⁺.

28.21 g (94.88 mmol) 1-benzyl-4-(2,5-difluorophenyl)-1H-pyrrole-2-carbaldehyde together with 23.00 g (189.77 mmol) (R)-2-methylpropane-2-sulfinamide were placed in 403.0 mL absolute THF, mixed with 67.42 g (237.21 mmol) titanium(IV)isopropylate and stirred overnight at RT. 500.0 mL saturated NaCl solution and 1000.0 mL ethyl acetate were added and stirred for 1 h at RT. The solution was filtered through kieselguhr and the filtrate was washed twice with saturated NaCl solution. The organic phase was dried over magnesium sulfate, the solvent was evaporated under vacuum and the residue was purified using Biotage Isolera (silica gel, column 1500+340 g SNAP, flow rate 200 mL/min, ethyl acetate/cyclohexane 1:10).

LC-MS (Method 7): Rₜ=1.63 min; MS (ESipos): m/z=401 [M+H]⁺.

25.00 g (62.42 mmol) (R)—N-{(E/Z)[1-benzyl-4-(2,5-difluorophenyl)-1H-pyrrol-2-yl]-methylene}-2-methylpropane-2-sulfinamide were placed in absolute THF under argon and cooled to −78° C. Then 12.00 g (187.27 mmol) tert.-butyllithium (1.7 M solution in pentane) were added −78° C. and stirred at this temperature for 3 h. Then 71.4 mL Methanol and 214.3 mL saturated ammonium chloride solution were added in succession at −78° C. and the reaction mixture was allowed to warm to RT and stirred for 1 h at RT. It was diluted with ethyl acetate and washed with water. The organic phase was dried over magnesium sulfate and the solvent was evaporated under vacuum. The residue (R)—N-{(1R)-1-[1-Benzyl-4-(2,5-difluorophenyl)-1H-pyrrol-2-yl]-2,2-dimethylpropyl}-2-methylpropane-2-sulfinamide was used in the next synthesis step without further purification.

LC-MS (Method 6): Rₜ=2.97 min; MS (ESipos): m/z=459 [M+H]⁺.

28.00 g (61.05 mmol) (R)—N-{(1R)-1-[1-benzyl-4-(2,5-difluorophenyl)-1H-pyrrol-2-yl]-2,2-dimethyl propyl}-2-methylpropane-2-sulfinamide were placed in 186.7 mL 1,4-dioxane and then 45.8 mL HCl in 1,4-dioxane solution (4.0 M) were added. The reaction mixture was stirred for 2 h at RT and the solvent was evaporated under vacuum. The residue was purified by preparative RP-HPLC (column: Kinetix 100×30; flow rate: 60 mL/min, MeCN/water). The acetonitrile was evaporated under vacuum and dichloromethane was added to the aqueous residue. The organic phase was washed with sodium hydrogen carbonate solution and dried over magnesium sulfate. The solvent was evaporated under vacuum and the residue was dried under high vacuum. Yield: 16.2 g (75% of theoretical) of the title compound.

LC-MS (Method 6): Rₜ=2.10 min; MS (ESipos): m/z=338 [M-NH2]+, 709 [2M+H]⁺.

¹H-NMR (400 MHz, DMSO-d6): δ [ppm]=0.87 (s, 9H), 1.53 (s, 2H), 3.59 (s, 1H), 5.24 (d, 2H), 6.56 (s, 1H), 6.94 (m, 1H), 7.10 (d, 2H), 7.20 (m, 1H), 7.26 (m, 2H), 7.34 (m, 2H), 7.46 (m, 1H).

### Intermediate C58

### (2S)-4-[{(1R)-1-[1-Benzyl-4-(2,5-difluorophenyl)-1H-pyrrol-2-yl]-2,2 dimethyl propyl} (glycoloyl) amino]-2-({[2 (trimethylsilyl)ethoxy]carbonyl}amino)butanoic acid

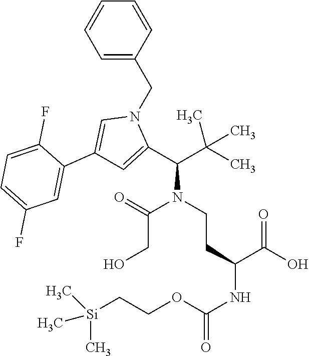 [SMILES: CC(C)(C)[C@H](c1cc(-c2cc(F)ccc2F)cn1Cc1ccccc1)N(CC[C@H](NC(=O)OCC[Si](C)(C)C)C(=O)O)C(=O)CO]

4.3 g (12.2 mmol) of intermediate C52 were dissolved in 525 mL DCM and 3.63 g (17.12 mmol) sodium triacetoxy borohydride plus 8.4 mL acetic acid were added. After stirring for 5 min at RT, 8.99 g (24.5 mmol) of intermediate L57 dissolved in 175 mL DCM was added and the reaction mixture was stirred for another 45 min at RT. The reaction mixture was then diluted with 300 mL DCM and washed twice with 100 mL sodium hydrogen carbonate solution and once with saturated NaCl solution. The organic phase was dried over magnesium sulfate, the solvent evaporated under vacuum and the residue dried under high vacuum. The residue was then purified by preparative RP-HPLC (column: Chromatorex C18). After purification of the corresponding fractions, the solvent was evaporated under vacuum and the residue dried under high vacuum. In this way, 4.6 g (61% of theoretical) methyl-(2S)-4-({(1R)-1-[1-benzyl-4-(2,5-difluorophenyl)-1H-pyrrol-2-yl]-2,2-dimethylpropyl}amino)-2-({[2-(trimethylsilyl)-ethoxy]carbonyl}amino)butanoate were obtained.

LC-MS (Method 12): Rₜ=1.97 min; MS (ESipos): m/z=614 (M+H)⁺.

2.06 g (3.36 mmol) of this intermediate were placed in 76 mL DCM and acylated with 0.81 mL (7.17 mmol) 2-chloro-2-oxoethylacetate in the presence of 2.1 mL triethylamine. After stirring for 20 h at RT, an additional 0.36 mL 2-chloro-2-oxoethyl acetate and 0.94 mL triethylamine were added and the reaction mixture stirred for an additional 15 min at RT. Then the reaction mixture was diluted with 500 mL ethyl acetate and washed twice in succession with 300 mL 5% citric acid, twice with 300 mL saturated sodium hydrogen carbonate solution and once with 100 mL saturated sodium chloride solution, then dried over magnesium sulfate and concentrated by evaporation. After drying under high vacuum, 2.17 g (79% of theoretical) of the protected intermediate were obtained.

LC-MS (Method 1): Rₜ=1.48 min; MS (ESipos): m/z=714 (M+H)⁺.

2.17 g (2.64 mmol) of this intermediate were dissolved in 54 mL THF and 27 mL water and 26 mL of a 2-molar lithium hydroxide solution were added. The reaction mixture was stirred for 30 min at RT and then adjusted to a pH between 3 and 4 with 1.4 mL TFA. The reaction mixture was concentrated under vacuum. After most of the THF was distilled off, the aqueous solution was extracted twice with DCM and then evaporated to dryness under vacuum. The residue was purified by preparative HPLC (column: Chromatorex C18). After combination of fractions, the solvent was evaporated under vacuum and the residue was lyophilized from acetonitrile/water. In this way, 1.1 g (63% of theoretical) of the title compound was obtained.

LC-MS (Method 1): Rₜ=1.34 min; MS (ESipos): m/z=656 (M−H)⁻.

¹H-NMR (400 MHz, DMSO-d6): δ [ppm]=0.03 (s, 9H), 0.58 (m, 1H), 0.74-0.92 (m, 1H), 1.40 (m, 1H), 3.3 (m, 2H), 3.7 (m, 1H), 3.8-4.0 (m, 2H), 4.15 (q, 2H), 4.9 and 5.2 (2d, 2H), 5.61 (s, 1H), 6.94 (m, 2H), 7.13-7.38 (m, 7H), 7.48 (s, 1H), 7.60 (m, 1H), 12.35 (s, 1H).

### Intermediate C61

### N-[(2S)-4-[{(1R)-1-[1-Benzyl-4-(2,5-difluorophenyl)-1H-pyrrol-2-yl]-2,2-dimethylpropyl}(glycoloyl)amino]-2-({[2-(trimethylsilyl)ethoxy]carbonyl}amino)butanoyl]-beta-alanine

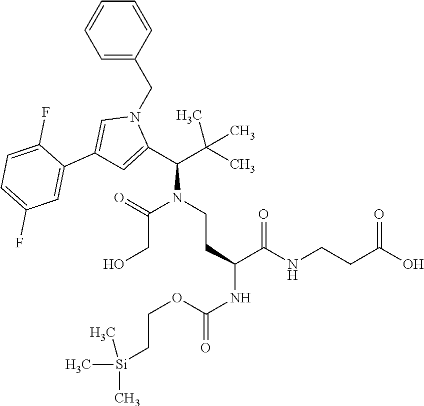 [SMILES: CC(C)(C)[C@H](c1cc(-c2cc(F)ccc2F)cn1Cc1ccccc1)N(CC[C@H](NC(=O)OCC[Si](C)(C)C)C(=O)NCCC(=O)O)C(=O)CO]

The title compound was prepared by coupling of 60 mg (0.091 mmol) intermediate C58 with methyl β-alaninate followed by ester cleavage with 2 M lithium hydroxide solution. This gave was 67 mg (61% of theoretical) of the title compound over 2 steps.

LC-MS (Method 1): Rₜ=1.29 min; MS (ESipos): m/z=729 (M−H)⁺.

### Intermediate C110(D)

### Dibenzyl-N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl-D-glutamate

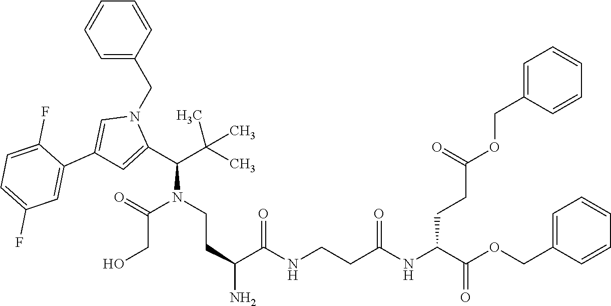 [SMILES: CC(C)(C)[C@H](c1cc(-c2cc(F)ccc2F)cn1Cc1ccccc1)N(CC[C@H](N)C(=O)NCCC(=O)N[C@H](CCC(=O)OCc1ccccc1)C(=O)OCc1ccccc1)C(=O)CO]

The title compound was by coupling of dibenzyl-D-glutamate, previously released from its p-toluenesulfonic acid salt by partitioning between ethyl acetate and 5% sodium hydrogen carbonate solution, with intermediate C61 in the presence of HATU and N,N-dipropyl-ethylamine and then splitting off the Teoc protective group with zin chloride in trifluoroethanol.

LC-MS (Method 1): Rₜ=1.08 min; MS (ESipos): m/z=894 [M+H]⁺.

### Intermediate L57

### Methyl-(2S)-4-oxo-2-({[2-(trimethylsilyl)ethoxy]carbonyl}amino)butanoate

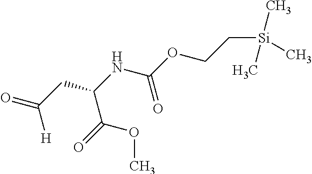 [SMILES: COC(=O)[C@H](CC=O)NC(=O)OCC[Si](C)(C)C]

500.0 mg (2.72 mmol) L-aspartic acid methyl ester hydrochloride and 706.3 mg (2.72 mmol) 2-(trimethylsilyl)ethyl-2,5-dioxopyrrolidin-1-carboxylate were placed in 5.0 mL 1,4-dioxane, and 826.8 mg (8.17 mmol) triethylamine were added. The reaction mixture was stirred overnight at RT. The reaction mixture was purified directly by preparative. RP-HPLC (column: Reprosil 250×40; 10μ, flow rate: 50 mL/min, MeCN/water, 0.1% TFA). The solvents were then evaporated under vacuum and the residue dried under high vacuum. This gave 583.9 mg (74% of theoretical) of the compound (3S)-4-methoxy-4-oxo-3-({[2-(trimethylsilyl)ethoxy]-carbonyl}-amino) butanoic acid.

LC-MS (Method 1): Rₜ=0.89 min; MS (ESlneg): m/z=290 (M−H)⁻.

592.9 mg (3 S)-4-Methoxy-4-oxo-3-({[2-(trimethylsilyl)ethoxy]carbonyl}amino)butanoic acid were placed in 10.0 mL 1,2-dimethoxyethane, cooled to −15° C., and 205.8 mg (2.04 mmol) 4-methylmorpholine and 277.9 mg (2.04 mmol) isobutyl chloroformate were added. The precipitate was filtered off by suction after 15 min and washed twice, each time with 10.0 mL 1,2-dimethoxyethan. The filtrate was cooled to −10° C., and 115.5 mg (3.05 mmol) sodium borohydride dissolved in 10 mL water were added with vigorous stirring. The phases were separated and the organic phase washed once with saturated sodium hydrogen carbonate solution and once with saturated NaCl solution. The organic phase was dried over magnesium sulfate, the solvent evaporated under vacuum and the residue dried under high vacuum. This gave 515.9 mg (91% of theoretical) of the compound methyl-N-{[2-(trimethylsilyl)ethoxy]carbonyl}-L-homoserinate.

LC-MS (Method 1): Rₜ=0.87 min; MS (ESipos): m/z=278 (M+H)⁺.

554.9 mg (2.00 mmol) methyl-N-{[2-(trimethylsilyl)ethoxy]carbonyl}-L-homoserinate were placed in 30.0 mL dichloromethane and 1.27 g (3.0 mmol) Dess-Martin periodinane and 474.7 mg (6.00 mmol) pyridine were added. The reaction mixture was stirred overnight at RT. After 4 h the reaction mixture was diluted with dichloromethane and the organic phase washed three times each with 10% Na₂S₂O₃ solution, 10% citric acid-solution and saturated sodium hydrogen carbonate solution. The organic phase was dried over magnesium sulfate and the solvent evaporated under vacuum. This gave 565.7 mg (97% of theoretical) of the title compound.

¹H-NMR (400 MHz, DMSO-d6): δ [ppm]=0.03 (s, 9H), 0.91 (m, 2H), 2.70-2.79 (m, 1H), 2.88 (dd, 1H), 3.63 (s, 3H), 4.04 (m, 2H), 4.55 (m, 1H), 7.54 (d, 1H), 9.60 (t, 1H).

### Intermediate L111

### N-(Pyridin-4-ylacetyl)-L-alanyl-N-methyl-L-alanyl-L-asparagine

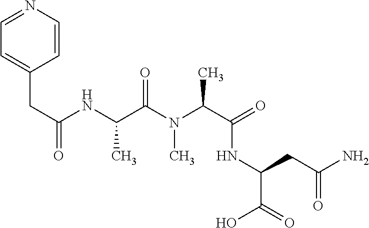 [SMILES: C[C@H](NC(=O)Cc1ccncc1)C(=O)N(C)[C@@H](C)C(=O)N[C@@H](CC(N)=O)C(=O)O]

The synthesis of the title compound was performed according to standard methods of peptide chemistry beginning with the HATU coupling of N-[(benzyloxy)carbonyl]-L-alanine with tert-butyl-N-methyl-L-alaninate hydrochloride salt in the presence of N,N-diisopropylethylamine and the deprotection of the carboxyl group with trifluoroacetic acid in DCM. This was followed by coupling with tert-butyl-L-asparaginate in the presence of HATU and N,N-diisopropylethylamine and then the hydrogenolytic splitting off of the Z protective group in DCM/methanol 1:1 over 10% palladium on active carbon at RT under hydrogen-normal pressure. Finally the intermediate was converted to the title compound by coupling with 4-pyridine-acetic acid in the presence of HATU and N,N-diisopropylethylamine and the deprotection of the carboxyl group with trifluoroacetic acid in DCM.

LC-MS (Method 1): Rₜ=0.16 min; MS (ESipos): m/z=408 (M+H)⁺.

### Intermediate L116

### N-[(Benzyloxy)carbonyl]-L-alanyl-N-methyl-L-alanine

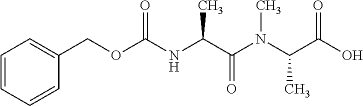 [SMILES: C[C@H](NC(=O)OCc1ccccc1)C(=O)N(C)[C@@H](C)C(=O)O]

The title compound was prepared starting from commercially available N-[(benzyloxy)carbonyl]-L-alanine using standard methods of peptide chemistry by coupling with tert-butyl-N-methyl-L-alaninate hydrochloride salt in the presence of HATU, and finally by splitting off the tert.-butyl ester protective group with TFA.

LC-MS (Method 1): Rₜ=0.68 min; MS (ESipos): m/z=309 [M+H]⁺

### Intermediate L117

### N-[(Benzyloxy)carbonyl]-L-alanyl-N-methyl-L-alanyl-L-asparagine-trifluoroacetic acid salt

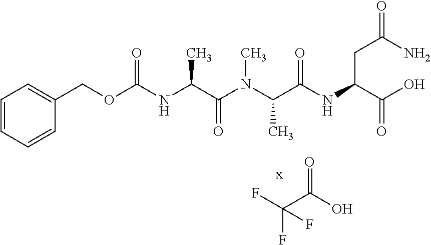 [SMILES: C[C@H](NC(=O)OCc1ccccc1)C(=O)N(C)[C@@H](C)C(=O)N[C@@H](CC(N)=O)C(=O)O ; O=C(O)C(F)(F)F]

The title compound was prepared starting from commercially available 4 tert-butyl-L-asparaginate using standard methods of peptide chemistry by coupling with N-[(benzyloxy)carbonyl]-L-alanyl-N-methyl-L-alanine (intermediate L116) in the presence of HATU, and finally by splitting off the tert.-butyl ester protective group with TFA.

LC-MS (Method 1): Rₜ=0.57 min; MS (ESineg): m/z=421 [M−H]⁻

### Intermediate O2

### N-{5-[(2,5-Dioxopyrrolidin-1-yl)oxy]-5-oxopentanoyl}-L-alanyl-N-methyl-L-alanyl-N¹-{(2S)-4-[{(1R)-1-[1-benzyl-4-(2,5-difluorophenyl)-1H-pyrrol-2-yl]-2,2-dimethylpropyl}(glycoloyl)amino]-1-[(3-{[(1R)-1,3-dicarboxypropyl]amino}-3-oxopropyl)amino]-1-oxobutan-2-yl}-L-aspartamide

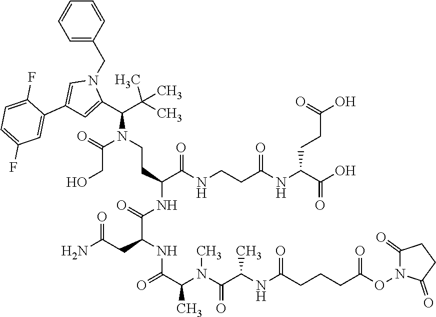 [SMILES: C[C@H](NC(=O)CCCC(=O)ON1C(=O)CCC1=O)C(=O)N(C)[C@@H](C)C(=O)N[C@@H](CC(N)=O)C(=O)N[C@@H](CCN(C(=O)CO)[C@@H](c1cc(-c2cc(F)ccc2F)cn1Cc1ccccc1)C(C)(C)C)C(=O)NCCC(=O)N[C@H](CCC(=O)O)C(=O)O]

The title compound was prepared starting from compound C110D first by coupling with intermediate L117 in the presence of HATU and N,N-diisopropylethylamine. In the next step all protective groups were removed by 1-hour hydrogenation over 10% palladium on active carbon in DCM-Methanol 1:1 under normal pressure hydrogen at RT and the deprotected intermediate then converted to the title compound by reacting with 1,1′-[(1,5-Dioxopentan-1,5-diyl)bis(oxy)]dipyrrolidin-2,5-dione in the presence of N,N-diisopropylethylamine. LC-MS (Method 1): Rₜ=0.93 min; MS (ESipos): m/z=1195 [M+H]⁺.

### B: Preparation Of Antibody/-Drug Conjugates (ADC)

### B-1. General Method for Generating Antibodies

The protein sequence (amino acid sequence) of the antibody used, for example TPP-14511, TPP-14509, TPP-14499, TPP-14505, TPP-14514, TPP-14495, TPP-10063 and 40C01 was converted to a DNA sequence encoding for the corresponding protein by a method known for the person skilled in the art and inserted into an expression vector suitable for the transient mammalian cell culture (as described by Tom et al., Chapter 12 in Methods Express: Expression Systems, edited by Micheal R. Dyson and Yves Durocher, Scion Publishing Ltd, 2007).

### B-2. General Method for Expression of Antibodies in Mammalian Cells

The antibodies, for example TPP-14511, TPP-14509, TPP-14499, TPP-14505, TPP-14514, TPP-14495 and TPP-10063, were produced in transient mammalian cell cultures, as described by Tom et al., Chapter 12 in Methods Express: Expression Systems, edited by Micheal R. Dyson and Yves Durocher, Scion Publishing Ltd, 2007.

### B-3. General Method for Purification of Antibodies from Cell Supernatants

The antibodies, for example TPP-14511, TPP-14509, TPP-14499, TPP-14505, TPP-14514, TPP-14495 and TPP10063, were obtained from the cell culture supernatants. The cell supernatants were cleared of cells by centrifugation. Then the cell supernatants were purified by affinity chromatography on a MabSelect Sure (GE Healthcare) chromatography column. For this purpose the column was equilibrated in DPBS pH 7.4 (Sigma/Aldrich), the cell supernatant applied, and the column was washed with approx. 10 column volumes of DPBS pH 7.4+500 mM sodium chloride. The antibodies were eluted in 50 mM sodium acetate pH 3.5+500 mM sodium chloride and then further purified by gel filtration chromatography on a Superdex 200 column (GE Healthcare) in DPBS pH 7.4.

Commercially available antibodies were purified using standard chromatography methods from the commercial products (Protein A chromatography, preparative gel filtration chromatography (SEC—size exclusion chromatography)).

### B-4 General Method for Coupling to Lysine Side Chains

The following antibodies were used in the coupling reactions:

### Examples x: TPP-14495

### TPP-14499

### TPP-14505

### TPP-14509

### TPP-14511

TPP-14514

### TPP-10063

### 40C01

The coupling reactions were usually performed under argon.

To a solution of the appropriate antibody in PBS buffer in the concentration range between 1 mg/mL and 20 mg/mL, preferably about 10 mg/mL, depending on the desired loading, between 2 and 10 equivalents of the precursor compound to be coupled as a solution in DMSO were added. After stirring for 30 min to 6 h at RT the same quantity of precursor compound in DMSO was added again. In this process the volume of DMSO should not exceed 10% of the total volume. After an additional 30 min to 6 h of stirring at RT, the reaction mixture was applied to PD 10-columns (Sephadex® G-25, GE Healthcare) equilibrated in PBS and eluted with PBS buffer. After purification over the PD10 column, in each case solutions of the appropriate ADC in PBS buffer were obtained. Then concentration was performed by ultracentrifugation and the sample optionally rediluted with PBS buffer. If necessary for better removal of low-molecular-weight components, concentration by ultrafiltration was repeated after redilution with PBS buffer. For biological tests, as needed, concentrations in the range of 0.5-15 mg/mL were established in the final ADC samples by redilution.

The respectively specified protein concentration for the ADC solution in the exemplary embodiment was determined. In addition, the loading of the antibody (active agent/mAb ratio) was detected using the methods described under B-6.

AK₂ has the following significance in the structural formulas shown

Examples x: TPP-14495—NH§ ²

### TPP-14499—NH§ ²

### TPP-14505—NH§ ²

### TPP-14509—NH§ ²

### TPP-14511—NH§ ²

TPP-14514—NH§ ²

### TPP-10063—NH§ ²

### 40C01—NH§ ²

where
§ ² represents the bond with the carbonyl group.
and
   NH represents the side-chain amino group of a lysine residue of the antibody.

### Further Purification and Characterization of the Conjugates According to the Invention

After reaction took place, in some cases the reaction mixture was concentrated, for example by ultrafiltration, and then desalinated and purified by chromatography, for example on a Sephadex® G-25. The elution was performed, for example, with phosphate-buffered saline solution (PBS). Then the solution is sterile-filtered and frozen. Alternatively, the conjugate can be lyophilized.

### B-5. Checking the Antigen Binding of the ADC

The binding capacity of the binder to the target molecule was checked after coupling was performed. Many methods for this are known to the person skilled in the art. For example, the affinity of the conjugate can be checked using ELISA technology surface plasmon resonance analysis (BIAcore™ measurements). The person skilled in the art can measure the conjugate concentration using conventional methods, for example for antibody conjugates by protein determination (see also Doronina et al.; Nature Biotechnol. 2003; 21:778-784 and Polson et al., Blood 2007; 1102:616-623).

### B-6. Determination of Antibody and Toxophore Loading

The toxophore loading (designated as DAR, drug-to-antibody ratio in the tables) of the conjugates in the PBS buffer solutions obtained as described in the exemplary embodiments was determined as follows:

The toxophore loading of the antibody (DAR) was determined, independent of the binding site, by UV absorption during size exclusion chromatography (SEC), abbreviated in the following as SEC-UV. For this purpose, 50 μL of the ADC was analyzed by SEC. The analysis was performed on an Agilent 1260 HPLC system with detection at 280 nm and detection at 260 nm. A Superdex 200 10/300 GL column from GE Healthcare (Lot No: 10194037) (10×310 mm, 1 μm particle size) with a flow rate of 1 ml/min under isocratic conditions was used. The mobile phase consisted of PBS buffer (pH 7.2). For determining the active agent load from the HPLC chromatogram, the ratio R of the peak areas of the monomer peaks at 260 nm and at 280 nm was determined. The drug load (DAR) was determined from this as follows:

$$DAR = \frac{\text{?} - R \cdot \text{?}}{R \cdot \text{?} - \text{?}}$$

$$\text{?} \text{indicates text missing or illegible when filed}$$

Here, ε represents the molar extinction coefficients of the antibody (Ab) and the drug (D). λ_{drug} represents the wavelength at 260 nm, whereas 280 represents 280 nm. The extinction coefficients of the antibodies at 280 nm and at 260 nm were determined experimentally. The mean value of these determinations for various antibodies was used for the DAR calculation. The molar extinction coefficients at 280 nm and at 260 nm were also determined experimentally for the KSP toxophore. The following wavelengths and extinction coefficients were used for the DAR calculations:

| | | ε(280 nm) | ε(260 nm) |
|---|---|---|---|
| | (λ_{drug}) (nm) | [1/μM] | [1/μM] |
| Antibody | | 0.2284 | 0.1163 |
| KSP | 260 | 0.010 | 0.014 |

The concentration of the ADCs was determined by measuring the U V absorption at 280 nm. The concentration was determined via the molar absorption coefficient of the respective antibody. In order to also consider the absorption of the toxophore at 280 nm, the concentration measured at 280 nm was corrected using the following equation:

concentration=preliminary concentration/(1+DARuv*(□Toxophore 280 nm/□Antibody 280 nm))

Here, “preliminary concentration” represents the concentration calculated using only the absorption coefficients of the antibody, DARuv is the DAR of the respective ADC determined by SEC-UV, and □ Toxophore 280 nm and □ Antibody 280 nm are the respective extinction coefficients of the toxophore and the antibody at 280 nm.

In some cases, the DAR determination of lysine-linked ADCs was also performed by mass spectrometric determination of the molecular weights of the individual conjugate species. This also allowed confirmation of the antibody and the coupled linker-toxophore species. For this, first the antibody conjugates were deglycosylated with PNGaseF, the sample acidified and after HPLC separation/desalination, were analyzed mass spectrometrically by ESI-MicroTofQ (Bruker Daltonik). All spectra over the signal in the TIC (Total Ion Chromatogram) were added together and the Molecular weight of the various conjugate species were calculated based on MaxEnt deconvolution. After signal integration of the various species the DAR(=Drug/Antibody ratio) was then calculated. For this purpose the sum of the toxophore number-weighted integration result of all species was divided by the sum of the simply weighted integration results for all species.

The protein identification was performed prior to coupling. In addition to the molecular weights determination following deglycosylation and/or denaturation, for this purpose tryptic digestion was performed, and after denaturation, reduction and derivatization, the identity of the protein was confirmed on the basis of the tryptic peptide demonstrated.

### Exemplary Embodiments of Metabolites

### Example M1

### N-{(2S)-2-Amino-4-[{(1R)-1-[1-benzyl-4-(2,5-difluorophenyl)-1H-pyrrol-2-yl]-2,2-dimethyl propyl}(glycoloyl)amino]butanoyl}-beta-alanyl-D-glutamic acid

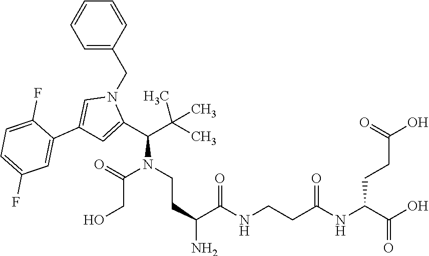 [SMILES: CC(C)(C)[C@H](c1cc(-c2cc(F)ccc2F)cn1Cc1ccccc1)N(CC[C@H](N)C(=O)NCCC(=O)N[C@H](CCC(=O)O)C(=O)O)C(=O)CO]

Intermediate C110D was converted into the title compound by 1-hour hydrogenation over 10% palladium on active carbon in ethanol under normal pressure hydrogen at RT.

LC-MS (Method 1): Rₜ=1.78 min; MS (ESipos): m/z=714 [M+H]⁺.

The ADCs shown below as examples can release the preferred metabolites M1, which has preferred pharmacologic properties.

### Exemplary Embodiments—ADCs

### Example 1

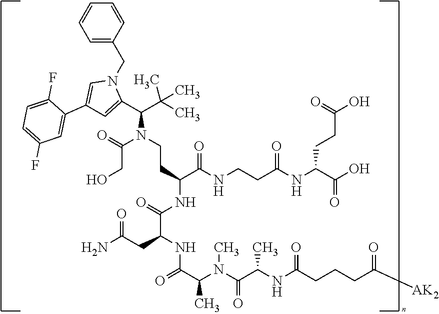

### Exemplary Procedure A

To 2.9 mg of the antibody in question in 0.3 mL PBS (c=10 mg/mL), under argon, 10 Eq (0.2 mg) of intermediate Q2 dissolved in 30 μL DMSO were added. After stirring for 1 h at RT, once again the same amount was added and the reaction mixture was stirred for an additional hour at RT. Then the reaction mixture was diluted with PBS buffer (pH7.2) to 2.5 mL, purified over a Sephadex column purified and then concentrated by ultracentrifugation and rediluted with PBS (pH7.2).

### Exemplary Procedure B

To 60 mg of the antibody in question in 6 mL PBS buffer (pH7.2) (c=10 mg/mL) under argon, 10 Eq (4.78 mg) of intermediate Q2 dissolved in 3004 DMSO was added. Then the reaction mixture, diluted to 10 mL with PBS buffer (pH7.2), was purified over a Sephadex column and then concentrated by ultracentrifugation, rediluted with PBS (pH7.2), reconcentrated and sterile-filtered.

| | | | C | |
|---|---|---|---|---|
| Example | Antibody | Procedure | [mg/mL] | DAR |
| 1x-14495 | TPP-14495 | B | 7.99 | 6.2 |
| 1x-14499 | TPP-14499 | B | 8.95 | 5.4 |
| 1x-14505 | TPP-14505 | A | 1.5 | 3.8 |
| 1x-14509 | TPP-14509 | B | 8.19 | 5.1 |
| 1x-14511 | TPP-14511 | B | 10.37 | 5.4 |
| 1x-14514 | TPP-14514 | A (1.9 mg AK) | 1.26 | 3.7 |
| 1x-10063 | TPP-10063 | A (5 mg AK) | 1.69 | 4.2 |
| 1x-40C01 | 40C01 | A (2.5 mg AK) | 1.87 | 5.3 |

The Following ADCs were Prepared for Comparison Purposes:

### Reference Example R1

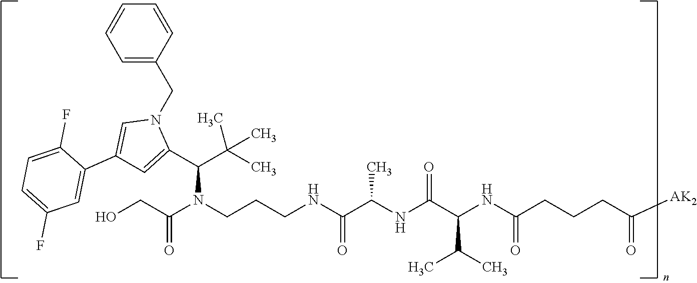

ADCs of this type were disclosed in WO2015/096982 and in WO2016/096610 with various antibodies, including, for example cetuximab and trastuzumab. For comparison, the precursor intermediate F194 disclosed therein was furthermore also reacted with the anti-CXCR5 antibodies TPP-14495, TPP-14499, TPP-14509 and TPP-14511. The following ADCs were used for comparison purposes:

| | antibody | C | |
|---|---|---|---|
| example | TPP- | [mg/mL] | DAR |
| Rlx-14495 | 14495 | 0.71 | 1.8 |
| Rlx-14499 | 14499 | 3.37 | 2.4 |
| Rlx-14509 | 14509 | 3.06 | 1.8 |
| Rlx-14511 | 14511 | 2.01 | 3.7 |

For the reference example R1 in WO2015/096982 the metabolite example 98 formed from it was described; it will be shown here as reference example RIM.

### Reference Example R1M

### N-(3-Aminopropyl)-N-{(IR)-1-[1-benzyl-4-(2,5-difluorophenyl)-IH-pyrrol-2-yl]-2,2-dimethyl propyl}-2-hydroxyacetamide

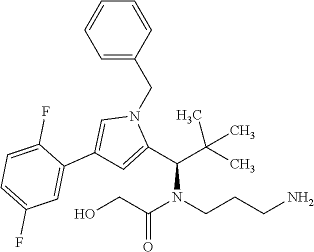 [SMILES: CC(C)(C)[C@H](c1cc(-c2cc(F)ccc2F)cn1Cc1ccccc1)N(CCCN)C(=O)CO]

The preparation was performed as described in WO2015/096982 as example 98.

The biologic data for these reference compounds, which were disclosed in the said application or obtained with the new reference compounds, will be described in section C.

### C: Evaluation Of Biological Activity

The biological activity of the compounds according to the invention can be demonstrated with the assays described below

### a. c-1a Determination of the Cytotoxic Activity of the ADCs

The analysis of the cytotoxic activity of the ADCs is performed on various cell lines:

Rec-1: human mantle cell lymphoma cells (B cell non-Hodgkin's lymphoma) ATCC CRL-3004, Standard medium: RPMI 1640 (Gibco, No. 21875-034)+GlutaMAX I (Invitrogen 61870)+10% FCS superior (Biochrom, No. S0615).) CXCR5-positive
HBL-1: human B cell lymphoma cells (diffuse large B-cell lymphoma) ATT CRL-RRID (Resource Identification Initiative): CVCL_4213, first described in Abe et al. Cancer 61:483-490(1988), obtained by Prof Lenz, University of Munster; standard medium: RPMI 1640 (Biochrom; #FG1215, stab. glutamine)+10% FCS (Biochrom; #S0415), culturing analogous to Rec-I cells; CXCR5 positive
NCI-H292: human mucoepidermoid lung cancer cells, ATCC-CRL-1848, standard medium: RPMI 1640 (Biochrom; #FG1215, stab. glutamine)+10% FCS (Sigma #F2442), TWEAKR-positive; EGFR-positive.
Oci-Ly-1: human B-cell lymphoma cells (B cell non-Hodgkin's lymphoma, assigned to germinal center B-cell like subtype), DSMZ ACC-722, standard medium: IMDM (Gibco No 31980-22)+20% FCS superior (Biochrom, No. S0615); CXCR5 positive.

SU-DHL-6: human B cell lymphoma cells (B cell non-Hodgkin, described as diffuse, mixed small and large cell type; cell line) ATCC-CRL-2959, standard medium: RPMI-1640 High Glucose (ATCC 30-2001) with L-glutamine, Hepes, sodium pyruvate+10% FCS (FBS Gibco 10500-064 heat inactivated, EU approved), CXCR5 positive.

The culturing of the cells is performed according to the standard method, as specified at the American Tissue Culture Collection (ATCC) or the Leibniz-Institut DSMZ-Deutsche Sammlung von Mikroorganismen and Zellkulturen GmbH (DSMZ) for the respective cell lines.

### CTG Assay

The cells were cultured using the standard method, with the growth media specified under C-1. To perform the test, the suspended cells were counted and seeded in a 96-well culture plate with a white background (Perkin Elmer, NO 10775584) (at 75 μL/well; the resulting cell numbers per well are: Rec-1: 3000 cells/well, HBL-1 and Oci-Ly-1: 6000 cells/well) and incubated in an incubator at 37° C. and 5% carbon dioxide. After 24 h the antibody-active agent conjugates in 254 culture medium (concentrated four-fold) were applied to the cells, so that final concentrations of the antibody-active agent conjugates of 3×10⁻⁷ M to 3×10⁻¹² M were reached on the cells (triplicate). Then the cells were incubated in an incubator at 37° C. and 5% carbon dioxide. In a parallel plate, the cell vitality was determined at the beginning of the active agent treatment (day 0) with the Cell Titer Glow (CTG) Luminescent Cell Viability Assay (Promega #G7573 and #G7571). For this purpose, 100 μL of the substrate were added per cell batch; then the plates were covered with aluminum foil, shaken for 2 minutes at 180 rpm with the plate shaker, allowed to stand for 8 minutes on the laboratory bench and then measured with a luminometer (Victor X2, Perkin Elmer). The substrate detected the ATP content in the living cells, generating a luminescence signal whose height is directly proportional to the vitality of the cells. After 72 h of incubation with the antibody-active agent conjugates, the vitality of these cells was also determined using the Cell Titer Glow Luminescent Cell Viability Assay as described above. From the measured data, the IC₅₀ of the growth inhibition was calculated compared to untreated cells and to Day 0 using the DRC (Dose Response Curve) Analysis Spreadsheets based on 4-parameter fitting. The DRC Analysis Spreadsheet is a Biobook Spreadsheet developed by Bayer Pharma AG and Bayer Business Services on the IDBS E-WorkBook Suite platform (IDBS: ID Business Solutions Ltd., Guildford, UK).

### MTT Assay

The culturing of the cells was performed according to the standard method with the growth media specified under C-1. For execution, the cells were separated with a solution of Accutase in PBS (Biochrom AG #L2143), pelleted, resuspended in culture medium, counted and seeded on a 96-well culture plate with a white background (Costar #3610) (NCI H292: 2500 cells/well; in 100 μL total volume). Then the cells were incubated in an incubator at 37° C. and 5% carbon dioxide. After 48 h a change of medium was performed. Then the antibody-active agent conjugates in 10 μL culture medium at concentrations of 10⁻⁵ M to 10⁻¹³ M were pipetted onto the cells (triplicate), before the mixture was incubated in the incubator at 37° C. and 5% carbon dioxide. The cells in the suspension were counted and seeded into a 96-well culture plate with white background (Costar #3610) (#3610) (Rec-1: 3000 cells/well at a total volume of 100 μL). After 6 hours of incubation in the incubator at 37° C. and 5% carbon dioxide, the medium was changed and the antibody-active agent conjugates or metabolites in 10 μL culture medium in concentrations from 10⁻⁵ M to 10⁻¹³ M were pipetted onto the cells (triplicate) in 90 μL. The reaction mixture was incubated in the incubator at 37° C. and 5% carbon dioxide. After 96 h the cell proliferation was detected using the MTT assay (ATCC, Manassas, Va., USA, Catalog No. 30-1010K). For this the MTT reagent was incubated with the cells for 4 h, before lysis of the cells was performed overnight by adding the detergent. The color formed was detected at 570 nm (Infinite M1000 pro, Tecan). Based on the measured data the IC₅₀ of the growth inhibition was calculated using the DRC (dose-response curve). The proliferation without the test substance, but with otherwise identically treated cells, is defined as the 100% value.

In Table la below the IC₅₀ values of representative exemplary embodiments from this assay are presented:

TABLE 1a

| | Rec-1 | HBL1 | Oci-Ly-1 | Rec-1 |
|---|---|---|---|---|
| | IC₅₀ | IC₅₀ | IC₅₀ | IC₅₀ |
| Example | [M] CTG | [M] CTG | [M] CTG | [M] MTT |
| 1x-14495 | 1.09E−08 | 8.30E−08 | 5.llE−10 | n.d. |
| 1x-14499 | 8.29E−09 | 5.33E−08 | 2.80E−10 | 2.5E−09 |
| 1x-14505 | 1.25E−08 | 9.35E−08 | 1.96E−10 | 2.6E−09 |
| 1x-14509 | 3.06E−09 | 2.71E−08 | 2.06E−10 | 8.3E−10 |
| 1x-14511 | 9.80E−09 | 7.96E−08 | 4.35E−10 | 2.9E−09 |
| 1x-14514 | 2.03E−08 | 1.41E−07 | 6.26E−10 | 8.7E−10 |
| 1x-10063 | 1.00E−08 | 4.54E−08 | 4.llE−10 | 2.1E−08 |
| 1x-40C01 | 9.62E−08 | >3.00E−07 | | 3.3E−08 |

In Table 1b below, the IC₅₀ values of the reference examples from this assay are presented.

TABLE 1b

| | Rec-1 IC₅₀ |
|---|---|
| Example | [M] CTG |
| R1x-14495 | >3.00E−07 |
| R1x-14499 | >3.00E−07 |
| R1x-14509 | >3.00E−07 |
| R1x-14511 | >3.00E−07 |

The specified activity data relate to the exemplary embodiments with the specified active agent/mAB ratios described in the present experimental section. The values may differ at other active agent/mAB ratios. The IC50 values are mean values from several independent experiments or single values. The efficacy of the antibody-active agent conjugates was selective versus the respective isotype control, which contained the respectively appropriate linker and toxophore.

The ADCs according to the invention generally exhibit a distinctly improved cytotoxic potency over the corresponding reference examples.

### C-1b Determination of the Inhibition of the Kinesin Spindle Protein KSP/Eg5 Using Selected Examples

The motor domain of the human kinesin spindle protein KSP/Eg5 (tebu-bio/Cytoskeleton Inc, No. 027EG01-XL) was incubated in a concentration of 10 nM with microtubules (bovine or porcine, tebu-bio/Cytoskeleton Inc) stabilized with 50 μg/ml taxol (Sigma No. T7191-5MG) for 5 min at RT in 15 mM PIPES, pH 6.8 (5 mM MgCl₂ and 10 mM DTT, Sigma). The freshly prepared mixture was aliquoted into a 384 MTP (from Corning). This was followed by the addition of the inhibitors to be investigated at concentrations from 1.0×10⁻⁶ M to 1.0×10⁻¹³ M and ATP (final concentration 500 μM; Sigma). The mixture was incubated at RT for 2 h. The ATPase activity was determined by detection of the inorganic phosphate produced with malachite green (Biomol). Addition of the reagent was followed by incubation for 50 min at RT before the absorption was detected at a wavelength of 620 nm. Monastrol (Sigma, M8515-1 mg) and ispinesib (AdooQ Bioscience A10486) were used as positive controls. The individual data for the dose-efficacy curve are from eight-fold determinations. The IC₅₀ values are mean values from two independent experiments. The 100% control was the sample that had not been treated with inhibitors.

Table 2 below summarizes the IC₅₀ values of representative exemplary embodiments from the assay described and the corresponding cytotoxicity data (MTT assay):

TABLE 2

| | KSP Assay | NCI-H292 | Rec-1 | Rec-1 |
|---|---|---|---|---|
| | IC₅₀ | IC₅₀ | IC₅₀ | IC₅₀ |
| Examples | [M] | [M] MTT | [M] MTT | [M] CTG |
| M1 | 1.59E−09 | 1.74E−07 | 3.87E−07 | 3.09E−07 |
| R1M | 1.09E−09 | 2.70E−10 | 2.93E−10 | 2.05E−10 |

The activity data presented relate to the exemplary embodiments described in the present experimental section.

### C-1c Enzymatic Assays

### Legumain Assay

The legumain assay was performed with recombinant human enzyme. The rhlegumain enzyme solution (Catalog #2199-CY, R&D Systems) was diluted to the desired concentration in 50 mM Na acetate buffer/100 mM NaCl, pH4.0, and preincubated for 2 h at 37° C. The rhlegumain was then adjusted to a final concentration of 1 ng/4 in 50 mM MES buffer, 250 mM NaCl, pH 5.0. For each legumain-cleavable prodrug to be investigated, a reaction mixture was made up in a micro-reaction vessel (0.5 ml, Eppendorf). For this, the substrate solution was adjusted with 50 mM MES buffer, 250 mM NaCl, pH 5.0 to the desired concentration (2-fold concentration). For the kinetic measurement of the enzymatic reaction, first 250 μL of the legumain solution was taken and the enzymatic reaction was started by adding 250 μL of the substrate solution (final concentration, single concentration; 3 μM). Samples of 50 μL each were taken at various times. Immediately, 100 μL ice-cold methanol was added to the sample to stop the enzymatic reaction and then frozen at −20° C. The selected sampling times were after 0.5 h, 1 h, 3 h and 24 h. The samples were then examined by RP-HPLC analysis and by LC-MS. The determination of the toxophore released enabled the determination of the half-time t_{1/2} of the enzymatic reaction.

As a representative example for demonstrating the legumain-mediated dissociation, the model compound was prepared as the substrate for the legumain assay.

### Reference Example Model Compound A

### lo N-(Pyridin-4-ylacetyl)-L-alanyl-N-methyl-L-alanyl-N¹-(2S)-4-[{(IR)-1-[1-benzyl-4-(2,5-difluorophenyl)-IH-pyrrol-2-yl]-2,2-dimethylpropyl}(glycoloyl)amino]-1-(methylamino)-1-oxobutan-2-yl]-L-aspartamide

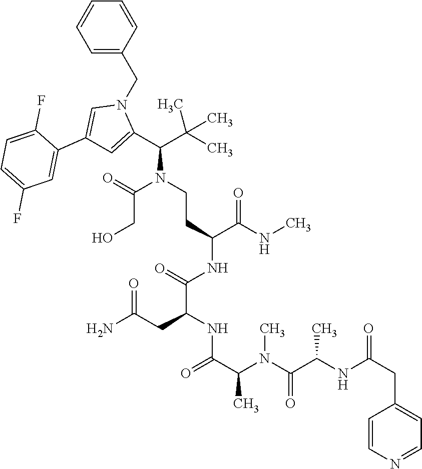 [SMILES: CNC(=O)[C@H](CCN(C(=O)CO)[C@@H](c1cc(-c2cc(F)ccc2F)cn1Cc1ccccc1)C(C)(C)C)NC(=O)[C@H](CC(N)=O)NC(=O)[C@H](C)N(C)C(=O)[C@H](C)NC(=O)Cc1ccncc1]

First, trifluoroacetic acid (2S)-2-amino-4-[{(1R)-1-[1-benzyl-4-(2,5-difluorophenyl)-1H-pyrrol-2-yl]-2,2-dimethylpropyl}(glycoloyl)amino]-N-methylbutanamide was prepared as described in WO 2015096982 A1. Then the title compound was prepared from this intermediate by coupling with intermediate L111 in DMF in the presence of HATU and N,N-diisopropylethylamine.

LC-MS (Method 1): Rₜ=0.83 min; MS (ESipos): m/z=916 [M+H]⁺.

Under the conditions described above, model compound A was split off from legumain with a half-life of 0.5 h.

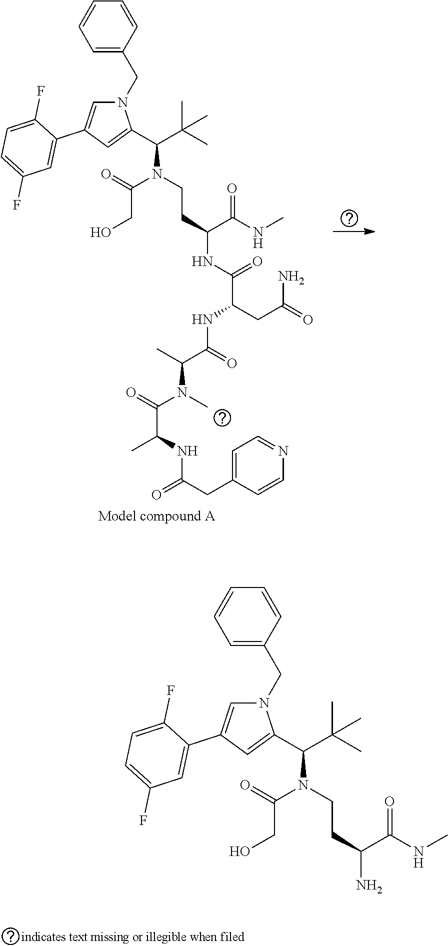 [SMILES: CNC(=O)[C@H](CCN(C(=O)CO)[C@@H](c1cc(-c2cc(F)ccc2F)cn1Cc1ccccc1)C(C)(C)C)NC(=O)[C@H](CC(N)=O)NC(=O)[C@H](C)N(C)C(=O)[C@H](C)NC(=O)Cc1ccncc1 ; CNC(=O)[C@@H](N)CCN(C(=O)CO)[C@@H](c1cc(-c2cc(F)ccc2F)cn1Cc1ccccc1)C(C)(C)C]

### C-2 Internalization Assay with Suspended Cells

Internalization is the key process for enabling specific and efficient preparation of the cytotoxic payload in antigen-expressing cancer cells by antibody-drug conjugates (ADC). This process is carried out by fluorescent labeling of specific antibodies and an isotype control antibody. For this purpose, first the conjugation of the fluorescent dye to lysine of the antibody was performed. The conjugation was conducted with a two-fold to 10-fold molar excess of CypHer 5E mono NHS ester (Batch 357392, GE Healthcare) at pH 8.3. After coupling was completed, the reaction mixture was purified by gel chromatography (Zeba Spin Desalting Columns, 40K, Thermo Scientific, No. 87768; elution buffer: DULBECCO'S PBS, Sima-Aldrich, No. D8537), to eliminate excess dye and adjust the pH. The protein solution was concentrated using VIVASPIN 500 columns (Sartorius stedim biotec). The determination of the dye load of the antibody was performed by spectrophotometric analysis (NanoDrop) followed by calculation (D/P=A_{dye}□ₚᵣₒₜₑᵢₙ:(A₂₈₀−0.16A_{dye})□_{dye}).

The dye load of the antibodies investigated here and the isotype control fell within comparable orders of magnitude. In cell binding assays tests were done to see that the coupling did not lead to any change in the affinity of the antibodies.

The antigen to be investigated is expressed by hematopoietic suspension cells, and therefore the internalization was examined in a FACS-based internalization assay.

Cells with various target expression levels were investigated. The cells (5×10⁴/well) were seeded in a 96-MTP (Greiner bio-one, CELLSTAR, 650 180, U-bottom) in 100 μL total volume. After addition of the target-specific antibody at a final concentration of 10 μg/ml, the reaction mixtures were incubated at 37° C. for different lengths of time (1 h, 2 h, 6 h, triplicate determination). The isotype check was handled under identical conditions. A parallel reaction mixture was kept constantly and incubated at 4° C. (negative control). The FACS analysis was performed using the Guava flow cytometer (Millipore). The kinetic evaluation was done by measuring the fluorescence intensity, and the assessment was conducted using the guavaSoft 2.6 software (Millipore). A significant and specific internalization was detected in various cells for the target-specific antibody described here. In these tests, the internalization of the antibodies TPP-14495, TPP14499, TPP-14505, TPP-14509, TPP-14511, TPP-14514 according to the invention was improved on Rec-1 and SU-DHL-6 cells, in contrast to TPP-10063 and 40C01 (TPP-14495 showed no improvement on SU-DHL-6). The isotype controls exhibited no internalization.

The observed fluorescence intensities (MFI) for the CXCR5 high-expressing Rec-1 cell line and the moderately CXCR5-expressing SU-DHL6 cell line are summarized in Table 3.

TABLE 3

| | Internalization Rec-1 | Internalization |
|---|---|---|
| Antibodies-example | [MFI] | SU-DHL-6 [MFI] |
| TPP-14495 | 84 | 12 |
| TPP-14499 | 122 | 55 |
| TPP-14505 | 135 | 61 |
| TPP-14509 | 129 | 51 |
| TPP-14511 | 99 | 24 |
| TPP-14514 | 134 | 53 |
| TPP-10063 | 65 | 22 |
| 40C01 | 49 | 13 |
| Isotype control | 2 | 1 |

### C-3 In Vitro Tests for Determining the Cell Permeability

The cell permeability of a substance can be studied by in vitro testing in a flux assay using Caco-2 cells [M. D. Troutman and D. R. Thakker, Pharm. Res. 20 (8), 1210-1224 (2003)].

For this purpose, the cells were cultured on 24-well filter plates for 15-16 days. To determine the permeation, the respective test substance in a HEPES buffer was placed on the cells either apically (A) or basally (B) and incubated for 2 h. After 0 h and after 2 h, samples were drawn from the cis- and trans-compartments. The samples were separated by HPLC (Agilent 1200, Boblingen, Germany) using reverse phase-columns. The HPLC system was coupled over a turbo ion spray interface to an API 4000 triple quadrupole mass spectrometer (AB SCIEX Deutschland GmbH, Darmstadt, Germany). The permeability was evaluated based on a Pₐₚₚ value, which was calculated using the formula published by Schwab et al. [D. Schwab et al., J Med. Chem. 46, 1716-1725 (2003)]. A substance was classified as actively transported if the ratio of Pₐₚₚ (B-A) to Pₐₚₚ (A-B) (efflux ratio) was >2 or <0.5.

Of decisive importance for toxophores, released intracellularly are the permeability from B to A [Pₐₚₚ (B-A)] and the ratio from Pₐₚₚ (B-A) to Pₐₚₚ (A-B) (efflux ratio): the lower this permeability is, the more slowly are the active and passive transport processes of the of the substance by the monolayer of Caco-2 cells. so that after intracellular release the substance remains in the cell longer. This intracellular persistence of the metabolite increases the probability of interaction with the biochemical target (here: kinesin spindle protein, KSP/Eg5), which leads to improved cytotoxic efficacy.

Table 4 below shows permeability data of representative exemplified embodiments from this assay:

TABLE 4

| Exemplary | Pₐₚₚ (B − A) | |
|---|---|---|
| embodiment | [nm/s] | Efflux ratio |
| M1 | 2.7 | 1.6 |
| R1M | 213 | 16 |

The metabolite M1, which can be formed from the binder/active agent conjugates according to the invention, exhibits both a markedly reduced transport from the cell and a reduced efflux-ratio compared with the reference metabolite R1M, which can be formed from the binder/active agent conjugates of the reference example.

### C-4 In Vitro Tests for Determination of the Substrate Properties of P-Glycoprotein

### (P-gp)

Many tumor cells express transporter proteins for active compounds, which is frequently accompanied by development of resistance to cytostatics. Substances that are not substrates of such transporter proteins, such as P-glycoprotein (P-gp) or BCRP, therefore might exhibit an improved activity profile.

The substrate properties of a substance for P-gp (ABCB1) were determined with a flux assay using LLC-PK1 cells which overexpress P-gp (L-MDR1-cells) [A. H. Schinkel et al., J Clin. Invest. 96, 1698-1705 (1995)]. For this purpose the LLC-PKI- or L-MDR1 cells were cultured on 96-well filter plates for 3-4 days. To determine the permeation. the respective test substance, alone or in the presence of an inhibitor (such as ivermectin or verapamil) in a HEPES buffer was applied to the cells at either the apex (A) or the base (B) and incubated for 2 h. Samples were taken from the cis- and trans compartments after 0 h and after 2 h. The samples were separated by HPLC using reverse phase-columns. The HPLC system was coupled over a turbo ion spray interface to an API 3000 (Applied Biosystems Applera, Darmstadt, Deutschland) triple quadrupole mass spectrometer. The permeability was evaluated based on a Pₐₚₚ value calculated using the formula published by Schwab et al. [D. Schwab et al., J Med. Chem. 46, 1716-1725 (2003)]. A substance was classified as a P-gp substrate if the efflux ratio Pₐₚₚ (B-A) to Pₐₚₚ (A-B) was >2.

As additional criteria for evaluating the P-gp substrate properties, the efflux ratios in L-MDR1 and LLC-PKl cells or the efflux ratio in the presence or absence of an inhibitor may be compared with one another. If these values differ by more than a factor of 2, the substance in question is a P-gp substrate.

### C-5 Pharmacokinetics

The pharmacokinetic parameters of examples 1×-10063, 1×-14495, 1×-14499, 1×-14509 and 1×-14511 are determined in male Wistar rats. The substance to be investigated is administered as an intravenous solution. To simplify blood collection before administration of the substance, silicone catheters are placed in the right jugular vein of each animal. The surgical procedure is performed under isoflurane anesthesia at least one day before the experiment. After administration of the substance, blood is collected from the animals over a period of up to 168 hours. To collect the plasma, the samples are centrifuged in EDTA tubes and optionally stored at −20° C. until further processing. The pharmacokinetic characteristics of the ADCs such as clearance (CL), area under the curve (AUC) and terminal half-life (t112) are calculated from the recorded plasma concentration-time curves. The quantitation of the compounds was done using a suitable ELISA (enzyme-linked immunosorbent assay) method.

In Table 5 the pharmacokinetic parameters of examples 1×-10063, 1×-14495, 1×-14499, 1×-14509 and 1×-14511 are summarized.

TABLE 5

| Example | | 1x10063 | 1x-14495 | 1x-14499 | 1x-14509 | 1x-14511 |
|---|---|---|---|---|---|---|
| AUCₙₒᵣₘ | [kg* h/L] | 2515 | 3193 | 4063 | 2899 | 4292 |
| Clₘₐₜᵣᵢₓ | [mL/h/kg] | 0.4 | 0.31 | 0.25 | 0.34 | 0.23 |
| Vₛₛ | [L/kg] | 0.13 | 0.08 | 0.08 | 0.09 | 0.1 |
| t_{1/2} | [h] | 239 | 194 | 229 | 187 | 318 |

In this preliminary rat PK study after i.v. administration, a typical IgG profile was observed for all examples. No appreciable difference was found between the examples.

### Analysis for Quantitation of the ADCs Used

Der antibody fraction of the ADCs was determined by ligand binding assay (ELISA) as the total IgG concentration in plasma samples. The sandwich ELISA format was used. This ELISA is suitable for determining the concentrations of the ADCs in plasma and tumor samples. The ELISA plates were coated with goat anti-human-IgG-Fc antibodies. After incubation with the sample, the plates were washed and incubated with a detector conjugate from monkey anti-human-IgG(H+L) antibodies and horseradish peroxidase (HRP). After an additional washing step, the HRP substrate OPD was added and the color development followed via the absorption at 490 nm. Standard samples of known IgG concentration were fitted using 4-parameter equations. Between the lower (LLOQ) and upper (ULOQ) quantitation limits, the unknown concentrations were determined by interpolation.

### C5a: Identification of the ADC Metabolites after Internalization In Vitro

### Description of Method:

Internalization tests with immunoconjugates are performed to analyze metabolites produced intracellularly. For this purpose suitable tumor cells (3×10⁵/well) are seeded into 6-well plates and incubated overnight (37° C., 5% CO₂). Treatment is performed with 10 μg/mL (66 nM) of the substance to be investigated. The internalization was conducted at 37° C. and 5% CO₂. Cell samples are taken at various time points (0, 4, 24, 48, 72 h) for further analysis. First the supernatants (approx. 5 mL) are collected and following centrifugation (2 min, RT, 1000 rpm Heraeus Variofuge 3.0R), stored at −80° C. The cells are washed with PBS, separated with Accutase and the cell count taken. After washing again, a defined number of cells (2×10⁵) is mixed 100 mL lysis buffer (Mammalian Cell Lysis Kit (Sigma MCL1) and incubated under continuous shaking (Thermomixer, 15 min, 4° C., 650 rpm) in protein LoBind tubes (Eppendorf Cat. No. 0030 108.116). After incubation the lysate is centrifuged (10 min, 4° C., 12000 g, Eppendorf 5415R) and the supernatant collected. The supernatant obtained is stored at −80° C. All samples are then analyzed as follows.

To work up 50 μL of culture supernatant/cell lysate, this is mixed with 150 μL precipitation reagent (methanol) and shaken for 10 seconds. The precipitation reagent contains an internal standard (ISTD) at a suitable concentration (generally in the range of 20-100 μg/L). After centrifugation for 10 minutes at 1881 g, the supernatant is transferred to an Autosampler vial, made up with 300 μL of a buffer matched to the eluent and centrifuged for an additional 10 min at 1881 g.

Finally, measurement of the cell lysate and supernatant samples is performed using an HPLC-coupled API6500 triple quadrupole mass spectrometer from AB SCIEX Deutschland GmbH. For calibration, blank lysate or blank supernatant at appropriate concentration (0.1-1000 μg/L) is added. The limit of detection (LLOQ) is approx. 0.2 μg/L.

Quality controls for testing validity contain 4 and 40 μg/L.

### C5b: Identification of the ADC-Metabolites In Vivo

After i.v. administration of 3-30 mg/kg of various ADCs, the plasma and tumor concentrations of the ADCs as well as potentially occurring metabolites can be measured and the pharmacokinetic parameters such as clearance (CL), area under the curve (AUC) and half-life (t_{1/2}) can be calculated.

### Analysis for Quantitation of Potentially Occurring Metabolites

The measurement of the compounds in plasma, tumor, liver and kidney takes place after precipitation of the proteins, generally with methanol, using a high-pressure liquid chromatograph (HPLC) coupled with a triple quadrupole mass spectrometer (MS).

For workup of 50 μL plasma, this is mixed with 150 μL precipitation reagent (generally methanol) and shaken for 10 sec. The precipitation reagent contains an intimal standard (ISTD) at a suitable concentration (generally in the range of 20-100 μg/L). After centrifugation for 10 min at 1881 g, the supernatant is transferred into an autosampler vial, made up with 300 μL of a buffer matched to the mobile phase and shaken again.

In the workup of tumor or organ material, the respective material is mixed with 3-20 times its volume of extraction buffer. The extraction buffer contains 50 mL tissue protein extraction reagent (Pierce, Rockford, Ill.), two pellets of complete protease inhibitor cocktail (RocheDiagnostics GmbH, Mannheim, Germany) and phenyl methylsulfonyl fluoride (Sigma, St. Louis, Mo.) in a final concentration of 1 mM. The lysis and homogenization program of the Prescellys 24 lysis and homogenization apparatus (Bertin Technologies) is selected based on the tissue type (hard: tumor; soft: liver, kidney) (www.prescellys.com). The homogenized samples are allowed to stand overnight at 4° C. 50 μL of the homogenate are transferred into an autosampler vial and made up with 150 μL methanol containing ISTD, shaken for 10 sec, and then allowed to stand for 5 min. After addition of 300 μL ammonium acetate buffer (pH6.8) and brief shaking, the sample is centrifuged for 10 min at 1881 g.

For calibration for plasma samples, plasma is added, and for tissue samples, a blank matrix with concentrations of 0.6-1000 μg/L is added. The limit of detection (LOQ) is between 1 and 20 μg/L, depending on the sample type or tissue type.

Finally the plasma and matrix samples are measured on the HPLC-coupled API4500 triple quadrupole mass spectrometer from AB SCIEX Deutschland GmbH.

Quality controls for validity testing contain 4, 40 and 400 μg/L.

### C-6 Activity Test In Vivo

The activity of the conjugates according to the invention was tested in vivo, for example using xenograft models. The person skilled in the art is aware of methods in the prior art with which the activity of the compounds according to the invention can be tested (see e.g., WO 2005/081711; Polson et al., Cancer Res. 2009 Mar. 15; 69(6):2358-64). For example, rodents (e.g., mouse) were inoculated with a tumor cell line expressing the target molecule of the binder for this purpose. Then either a conjugate according to the invention, an isotype-antibody control conjugate or a control antibody or isotonic salt solution was administered to the inoculated animals. The administration was performed one or more times. After an incubation time of several days, the tumor sizes were determined for comparison between conjugate-treated animals and the control group. The tumors were smaller in the conjugate-treated animals.

### C-6a. Growth Inhibition/Regression of Experimental Tumors in the Mouse

Human tumor cells expressing the antigen for the antibody-active compound conjugate are inoculated subcutaneously into the flanks of immunosuppressed mice, for example NMRI nude mice or SCID mice. 1-10 million cells are separated from the cell culture, centrifuged and resuspended with medium or Matrigel. The cell suspension is injected under the skin of the mouse.

A tumor starts to grow at this site within a few days. Treatment is started after the tumor is established, approximately at a tumor size of 100 mm³. To investigate the efficacy on larger tumors, the treatment may also be started only at a tumor size of 200-500 mm³.

The treatment with ADCs is administered via the intravenous (i.v.) route into the tail vein of the mouse. The ADC is given in a volume of 5-10 mL/kg.

The treatment schedule depends on the pharmacokinetics of the antibody. With the conjugates according to the invention, the standard treatment schedule is once a week for 1-3 weeks. For earlier evaluation, a schedule of a single treatment may be suitable. However, the treatment may also be continued further, or a second cycle with three days of treatment can follow at a later time.

The standard method is to use 10-12 animals per treatment group. In addition to the groups that receive the active agent, one group is treated with the buffer according to the same schedule as a control group.

During the experiment, the tumor volume is regularly measured in two dimensions (length/width) using calipers. The tumor volume is determined according to (length×width²)/2. Comparison of the mean tumor volumes of the treatment group versus the control group is specified as % T/C volume specified. (% T/C=[mean tumor volume of treated group/mean tumor volume control group]×100.)

If all groups in the experiment are stopped at the same time after the end of treatment, the tumors can be removed and weighed. The comparison of the mean tumor weights of the treatment group with the control group is specified as % T/C weight (% T/C=[mean tumor weight of treated group/mean tumor weight of control group]×100.)

The response rate is evaluated as an additional efficacy endpoint. It corresponds to the number of mice with complete and partial tumor regressions after treatment (tumors at least 30% smaller than the size at the beginning of treatment, on a specified day).

### C-6b. Activity of the CXCR5 ADCs According to the Invention in Various Tumor Models

The tumor cells (e.g., REC-I, OCI-LYI) were inoculated subcutaneously in the flanks of female SCID mice (Janvier). At a mean tumor size/group of ˜280 mm³ intravenous treatment with the CXCR5-ADCs was administered. After the treatment, the tumor growth was optionally followed further in some cases.

The treatment with the CXCR5-ADCs according to the invention lead to a marked and sometimes long-lasting inhibition of tumor growth compared with the control group and the conjugated isotype control antibody. Table 7 shows the T/C values determined via the tumor volume on the respective day of the end of the study, calculated after the start of treatment.

TABLE 7

| | | | Dosing | % T/C | Response |
|---|---|---|---|---|---|
| Tumor model | Example | Dosage | schedule | volume^{a} | rate^{b} |
| REC-1 | lx-14495 | 10 mg/kg | QDxl | 3 | 10/10 |
| (human | lx-14499 | | | 4 | 9/10 |
| mantle cell | lx-14509 | | | 4 | 9/10 |
| lymphoma) | lx-14511 | | | 4 | 10/10 |
| OCI-LY1 | lx-14495 | 10 mg/kg | QDxl | 3 | 10/10 |
| (human | lx-14499 | | | 3 | 10/10 |
| DLBCL) | lx-14509 | | | 3 | 10/10 |
| | lx-14511 | | | 3 | 10/10 |

^{a}% T/C Volume, day 11 for REC-1, day 13 after treatment for OCI-LY-1,
^{b}Response rate, day 45 represents REC-1, day 41 after treatment for OCI-LY-1

All CXCR5-ADCs investigated exhibited a very high efficacy after a single treatment, with T/C

<10% and long-term tumor regression in 90-100% of mice.

1. Binder/active agent conjugates of general formula (I) 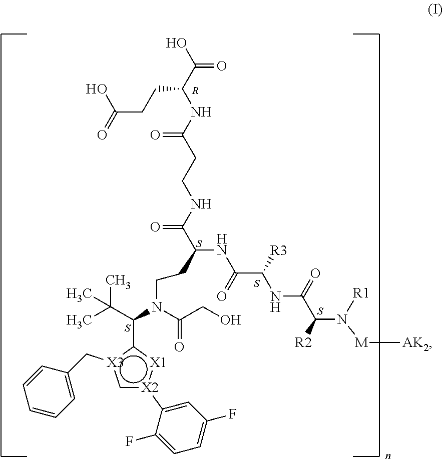
in which
X₁ represents N,
X₂ represents N and
X₃ represents C;
or
X₁ represents CH or CF,
X₂ represents C and
X₃ represents N;
or
X₁ represents NH,
X₂ represents C and
X₃ represents C,
or
X₁ represents CH,
X₂ represents N and
X₃ represents C,
R¹ represents hydrogen or methyl,
R² represents methyl, ethyl, —CH₂—CH(CH₃)₂, —CH₂—C(═O)OH or isopropyl,
R³ represents methyl, ethyl, —CH₂—CH(CH₃)₂ or —CH₂—C(═O)—NH₂,
M represents the group
#—C(═O)—CH(CH₃)—NH—C(═O)—(CH₂)₂₋₈—C(═O)-### or
#—C(═O)— (CH₂)₃—C(═O)-###,
n represents a number from 1 to 50,
AK₂ represents a binder or a derivative thereof, preferably an antibody or an antigen-binding fragment
# represents the bond to the active agent and
### represents the bond to an N atom of a lysine side chain of the binder,
as well as their salts and solvates thereof and their salts of these solvates.

2. The binder/active agent conjugates of general formula (I), according to claim 1, in which
X₁ represents CH,
X₂ represents C and
X₃ represents N,
R¹ represents hydrogen or methyl,
R² represents methyl, —CH₂—CH(CH₃)₂, —CH₂—C(═O)OH or isopropyl,
R³ represents methyl, ethyl, —CH₂—CH(CH₃)₂ or —CH₂—C(═O)—NH₂,
M represents the group
#—C(═O)—CH(CH₃)—NH—C(═O)—(CH₂)₃—C(═O)-### or
#—C(═O)—(CH₂)₃—C(═O)-###,
n represents a number from 1 to 50,
AK₂ represents a binder or a derivative thereof, preferably an antibody or an antigen-binding fragment,
# represents the bond to the active agent and
### represents the bond to an N-atom of a lysine side chain of the binder,
as well as their salts and solvates thereof and their salts of these solvates.

3. The binder/active agent conjugates of general formula (I), according to claims 1 and 2, in which
X₁ represents CH,
X₂ represents C and
X₃ represents N,
R¹ represents hydrogen or methyl,
R² represents methyl or isopropyl,
R³ represents methyl or —CH₂—C(═O)—NH₂,
M represents the group
#—C(═O)—CH(CH₃)—NH—C(═O)—(CH₂)₃—C(═O)-###,
n represents a number from 1 to 50,
AK₂ represents a binder or a derivative thereof, preferably for an antibody or an antigen-binding fragment,
# represents the bond to the active agent and
### represents the bond to an N-atom of a lysine side chain of the binder,
as well as their salts and solvates thereof and their salts of these solvates.

4. The binder/active agent conjugates of general formula (I), according to claims 1 to 3, in which
X₁ represents CH,
X₂ represents C and
X₃ represents N,
R¹ represents methyl,
R² represents methyl,
R³ represents —CH₂—C(═O)—NH₂,
M represents the group
#—C(═O)—CH(CH₃)—NH—C(═O)—(CH₂)₃—C(═O)-###,
n represents a number from 1 to 50,
AK₂ represents a binder or a derivative thereof, preferably an antibody or an antigen-binding fragment,
# represents the bond to the active agent and
### represents the bond to an N-atom of a lysine side chain of the binder,
as well as their salts and solvates thereof and their salts of these solvates.

5. The binder/active agent conjugates of formula (I), according to claims 1 to 4,
in which
R¹ represents methyl,
R² represents methyl,
R³ represents —CH₂—C(═O)—NH₂,
M represents the group
#—C(═O)—CH(CH₃)—NH—C(═O)—(CH₂)₃—C(═O)-###,
n represents a number from 1 to 20 and
AK₂ represents an antibody or an antigen-binding antibody fragment thereof,
# represents the bond to the active agent and
### represents the bond to an N-atom of a lysine side chain of the antibody or of the antigen-binding antibody fragment thereof,
as well as their salts and solvates thereof and their salts of these solvates.

6. The binder/active agent conjugates of formula (I), according to claims 1 to 4, of the structure 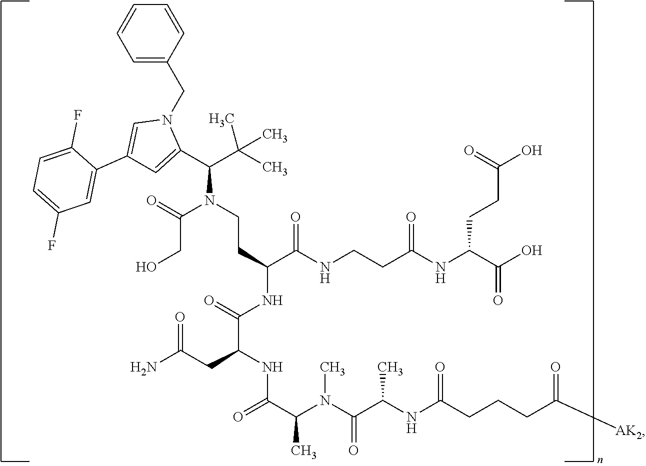
in which
AK₂ represents an antibody, bonded over an N atom of a lysine side chain and
n is 1 to 50,
as well as their salts and solvates thereof and their salts of these solvates.

7. The binder/active agent conjugates according to claim 6, in which
n is 1 to 20,
as well as their salts and solvates thereof and their salts of these solvates.

8. The binder/active agent conjugates according to claims 6 and 7, in which
n is 1 to 8,
as well as their salts and solvates thereof and their salts of these solvates.

9. The binder/active agent conjugates according to claims 6 to 8, in which
n is 4 to 8,
as well as their salts and solvates thereof and their salts of these solvates.

10. The binder/active agent conjugates according to claims 1 to 9, wherein
AK₂ represents an anti-CXCR5 antibody or an antigen-binding fragment thereof.

11. The binder/active agent conjugates according to claims 1 to 10, wherein
AK₂ represents an anti-CXCR5 antibody selected from the group consisting of TPP 14511, TPP 14509, TPP 14499, TPP 14505, TPP14514 and TPP14495, or represents an antigen-binding antibody fragment thereof.

12. The binder/active agent conjugates of general formula (I), according to claims 1 to 5 in which
R¹ represents methyl,
R² represents methyl,
R³ represents —CH₂—C(═O)—NH₂,
M represents the group
#—C(═O)—CH(CH₃)—NH—C(═O)—(CH₂)₃—C(═O)-###,
n represents a number from 1 to 20 and
AK₂ represents an anti-CXCR5 antibody selected from the group consisting of TPP 14511, TPP 14509, TPP 14499, TPP 14505, TPP 14514 and TPP 14495, or represents an antigen-binding antibody fragment thereof,
# represents the bond to the active agent and
### represents the bond to an N-atom of a lysine side chain of the antibody or of the antigen-binding antibody fragment thereof,
as well as their salts and solvates thereof and their salts of these solvates.

13. The binder/active agent conjugates according to claims 1 to 12, wherein AK₂
(i) represents an anti-CXCR5 antibody comprising a variable region of the heavy chain (VH) comprising the variable CDR1 sequence of the heavy chain (H-CDR1), as shown by SEQ ID NO: 2, the variable CDR2 sequence of the heavy chain (H-CDR2), as shown by SEQ ID NO: 3 and the variable CDR3 sequence of the heavy chain (H-CDR3), as shown by SEQ ID NO: 4, as well as a variable region of the light chain (VL) comprising the variable CDR1 sequence of the light chain (L-CDR1), as shown by SEQ ID NO: 6, the variable CDR2 sequence of the light chain (L-CDR2), as shown by SEQ ID NO: 7 and the variable CDR3 sequence of the light chain (L-CDR3), as shown by SEQ ID NO: 8,
(ii) represents an anti-CXCR5 antibody comprising a variable region of the heavy chain (VH) comprising the variable CDR1 sequence of the heavy chain (H-CDR1), as shown by SEQ ID NO: 12, the variable CDR2 sequence of the heavy chain (H-CDR2), as shown by SEQ ID NO: 13 and the variable CDR3 sequence of the heavy chain (H-CDR3), as shown by SEQ ID NO: 14, as well as a variable region of the light chain (VL) comprising the variable CDR1 sequence of the light chain (L-CDR1), as shown by SEQ ID NO: 16, the variable CDR2 sequence of the light chain (L-CDR2), as shown by SEQ ID NO: 17 and the variable CDR3 sequence of the light chain (L-CDR3), as shown by SEQ ID NO: 18,
(iii) represents an anti-CXCR5 antibody comprising a variable region of the heavy chain (VH) comprising the variable CDR1 sequence of the heavy chain (H-CDR1), as shown by SEQ ID NO: 22, the variable CDR2 sequence of the heavy chain (H-CDR2), as shown by SEQ ID NO: 23 and the variable CDR3 sequence of the heavy chain (H-CDR3), as shown by SEQ ID NO: 24, as well as a variable region of the light chain (VL) comprising the variable CDR1 sequence of the light chain (L-CDR1), as shown by SEQ ID NO: 26, the variable CDR2 sequence of the light chain (L-CDR2), as shown by SEQ ID NO: 27 and the variable CDR3 sequence of the light chain (L-CDR3), as shown by SEQ ID NO: 28,
(iv) represents an anti-CXCR5 antibody comprising a variable region of the heavy chain (VH) comprising the variable CDR1 sequence of the heavy chain (H-CDR1), as shown by SEQ ID NO: 32, the variable CDR2 sequence of the heavy chain (H-CDR2), as shown by SEQ ID NO: 33 and the variable CDR3 sequence of the heavy chain (H-CDR3), as shown by SEQ ID NO: 34, as well as a variable region of the light chain (VL) comprising the variable CDR1 sequence of the light chain (L-CDR1), as shown by SEQ ID NO: 36, the variable CDR2 sequence of the light chain (L-CDR2), as shown by SEQ ID NO: 37 and the variable CDR3 sequence of the light chain (L-CDR3), as shown by SEQ ID NO: 38,
(v) represents an anti-CXCR5 antibody comprising a variable region of the heavy chain (VH) comprising the variable CDR1 sequence of the heavy chain (H-CDR1), as shown by SEQ ID NO: 42, the variable CDR2 sequence of the heavy chain (H-CDR2), as shown by SEQ ID NO: 43 and the variable CDR3 sequence of the heavy chain (H-CDR3), as shown by SEQ ID NO: 44, as well as a variable region of the light chain (VL) comprising the variable CDR1 sequence of the light chain (L-CDR1), as shown by SEQ ID NO: 46, the variable CDR2 sequence of the light chain (L-CDR2), as shown by SEQ ID NO:47 and the variable CDR3 sequence of the light chain (L-CDR3), as shown by SEQ ID NO: 48, or
(vi) represents an anti-CXCR5 antibody comprising a variable region of the heavy chain (VH; SEQ ID NO: 51) comprising the variable CDR1 sequence of the heavy chain (H-CDR1), as shown by SEQ ID NO: 52, the variable CDR2 sequence of the heavy chain (H-CDR2), as shown by SEQ ID NO: 53 and the variable CDR3 sequence of the heavy chain (H-CDR3), as shown by SEQ ID NO: 54, as well as a variable region of the light chain (VL; SEQ ID NO:55) comprising the variable CDR1 sequence of the light chain (L-CDR1), as shown by SEQ ID NO: 56, the variable CDR2 sequence of the light chain (L-CDR2), as shown by SEQ ID NO: 57 and the variable CDR3 sequence of the light chain (L-CDR3), as shown by SEQ ID NO: 58,
or represents an antigen-binding fragment of these antibodies.

14. The binder/active agent conjugate according to any one of claims 1 to 12, wherein AK₂
(i) represents an anti-CXCR5 antibody comprising a variable region of the heavy chain (VH) as shown by SEQ ID NO: 1 as well as a variable region of the light chain (VL) as shown by SEQ ID NO: 5,
(ii) represents an anti-CXCR5 antibody comprising a variable region of the heavy chain (VH) as shown by SEQ ID NO: 11 as well as a variable region of the light chain (VL) as shown by SEQ ID NO: 15,
(iii) represents an anti-CXCR5 antibody comprising a variable region of the heavy chain (VH) as shown by SEQ ID NO: 21 as well as a variable region of the light chain (VL) as shown by SEQ ID NO: 25,
(iv) represents an anti-CXCR5 antibody comprising a variable region of the heavy chain (VH) as shown by SEQ ID NO: 31 as well as a variable region of the light chain (VL) as shown by SEQ ID NO: 35,
(v) represents an anti-CXCR5 antibody comprising a variable region of the heavy chain (VH) as shown by SEQ ID NO: 41 as well as a variable region of the light chain (VL) as shown by SEQ ID NO: 45, or
(vi) represents an anti-CXCR5 antibody comprising a variable region of the heavy chain (VH) as shown by SEQ ID NO: 51 as well as a variable region of the light chain (VL) as shown by SEQ ID NO: 55,
or represents an antigen-binding fragment of these antibodies.

15. The binder/active agent conjugate according to any one of claims 1 to 12, wherein AK₂
(i) represents an anti-CXCR5 antibody comprising a region of the heavy chain as shown by SEQ ID NO: 9 as well as a region of the light chain as shown by SEQ ID NO: 10,
(ii) represents an anti-CXCR5 antibody comprising a region of the heavy chain as shown by SEQ ID NO: 19 as well as a region of the light chain as shown by SEQ ID NO: 20,
(iii) represents an anti-CXCR5 antibody comprising a region of the heavy chain as shown by SEQ ID NO: 29 as well as a region of the light chain as shown by SEQ ID NO: 30,
(iv) represents an anti-CXCR5 antibody comprising a region of the heavy chain as shown by SEQ ID NO: 39 as well as a region of the light chain as shown by SEQ ID NO: 40,
(v) represents an anti-CXCR5 antibody comprising a region of the heavy chain as shown by SEQ ID NO: 49 as well as a region of the light chain as shown by SEQ ID NO: 50, or
(vi) represents an anti-CXCR5 antibody comprising a region of the heavy chain as shown by SEQ ID NO: 59 as well as a region of the light chain as shown by SEQ ID NO: 60, or represents an antigen-binding fragment of these antibodies.

16. The binder/active agent conjugate according to any one of claims 1 to 15, wherein the antibody or the antigen-binding antibody fragment binds to an extracellular target molecule.

17. The binder/active agent conjugate according to any one of claims 1 to 16, wherein the antibody or the antigen-binding antibody fragment binds to an extracellular cancer target molecule.

18. The binder/active agent conjugate according to any one of claims 1 to 17 wherein the antibody or the antigen-binding antibody fragment after binding to an extracellular target molecule internalizes on the target cell through binding the target cell.

19. A pharmaceutical composition comprising at least one binder/active agent conjugate according to one or more of the preceding claims in combination with an inert, nontoxic, pharmaceutically acceptable excipient.

20. The binder/active agent conjugate according to any one or more of the preceding claims for use in a method for treatment and/or prophylaxis of diseases.

21. The binder/active agent conjugate according to any one or more of the preceding claims for use in a method for treatment of hyperproliferative and/or angiogenic diseases.

22. The binder/active agent conjugate according to any one or more of the preceding claims for use in a method for treatment of cancer and tumors.

23. The binder/active agent conjugate according to any one or more of the preceding claims for use in a method for treatment of cancer and tumors in combination with one or more therapeutic compositions for cancer immunotherapy or with one or more active compounds directed against a molecular target from cancer immunotherapy.


SEQUENCE LISTING (81 sequences)

SEQ ID NO: 1 - protein, 118 aa, Artificial Sequence
  Description of Artificial Sequence Synthetic peptide
EVQLLESGGGLVQPGGSLRLSCAASGFTFSNYWMSWVRQAPGKGLEWVSAISGSGGSTRYADSVKGRFTISRDNSKNTLYLQMNSLRAEDTAVYYCTTQRRELGATNWGQGTLVTVSS

SEQ ID NO: 2 - protein, 5 aa, Artificial Sequence
  Description of Artificial Sequence Synthetic peptide
NYWMS

SEQ ID NO: 3 - protein, 17 aa, Artificial Sequence
  Description of Artificial Sequence Synthetic peptide
AISGSGGSTRYADSVKG

SEQ ID NO: 4 - protein, 9 aa, Artificial Sequence
  Description of Artificial Sequence Synthetic peptide
QRRELGATN

SEQ ID NO: 5 - protein, 111 aa, Artificial Sequence
  Description of Artificial Sequence Synthetic peptide
QSVLTQPPSASGTPGQRVTISCTGSSSNIGAGYVVHWYQQLPGTAPKLLIYSNNQRPSGVPDRFSGSKSGTSASLAISGLRSEDEADYYCQSYDSSLSGVVFGGGTKLTVL

SEQ ID NO: 6 - protein, 14 aa, Artificial Sequence
  Description of Artificial Sequence Synthetic peptide
TGSSSNIGAGYVVH

SEQ ID NO: 7 - protein, 7 aa, Artificial Sequence
  Description of Artificial Sequence Synthetic peptide
SNNQRPS

SEQ ID NO: 8 - protein, 11 aa, Artificial Sequence
  Description of Artificial Sequence Synthetic peptide
QSYDSSLSGVV

SEQ ID NO: 9 - protein, 447 aa, Artificial Sequence
  Description of Artificial Sequence Synthetic peptide
EVQLLESGGGLVQPGGSLRLSCAASGFTFSNYWMSWVRQAPGKGLEWVSAISGSGGSTRYADSVKGRFTISRDNSKNTLYLQMNSLRAEDTAVYYCTTQRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 217 aa, Artificial Sequence
  Description of Artificial Sequence Synthetic peptide
QSVLTQPPSASGTPGQRVTISCTGSSSNIGAGYVVHWYQQLPGTAPKLLIYSNNQRPSGVPDRFSGSKSGTSASLAISGLRSEDEADYYCQSYDSSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 118 aa, Artificial Sequence
  Description of Artificial Sequence Synthetic peptide
EVQLLESGGGLVQPGGSLRLSCAASGFTFSNYWGSWVRQAPGKGLEWVSAISGSGGSTYYADSVKGRFTISRDNSKNTLYLQMNSLRAEDTAVYYCTVLRRELGATNWGQGTLVTVSS

SEQ ID NO: 12 - protein, 5 aa, Artificial Sequence
  Description of Artificial Sequence Synthetic peptide
NYWGS

SEQ ID NO: 13 - protein, 17 aa, Artificial Sequence
  Description of Artificial Sequence Synthetic peptide
AISGSGGSTYYADSVKG

SEQ ID NO: 14 - protein, 9 aa, Artificial Sequence
  Description of Artificial Sequence Synthetic peptide
LRRELGATN

SEQ ID NO: 15 - protein, 111 aa, Artificial Sequence; identical to SEQ ID NO: 5
  Description of Artificial Sequence Synthetic peptide

SEQ ID NO: 16 - protein, 14 aa, Artificial Sequence
  Description of Artificial Sequence Synthetic peptide
TGSSSNIGAGYVVH

SEQ ID NO: 17 - protein, 7 aa, Artificial Sequence
  Description of Artificial Sequence Synthetic peptide
SNNQRPS

SEQ ID NO: 18 - protein, 11 aa, Artificial Sequence
  Description of Artificial Sequence Synthetic peptide
QSYDSSLSGVV

SEQ ID NO: 19 - protein, 447 aa, Artificial Sequence
  Description of Artificial Sequence Synthetic peptide
EVQLLESGGGLVQPGGSLRLSCAASGFTFSNYWGSWVRQAPGKGLEWVSAISGSGGSTYYADSVKGRFTISRDNSKNTLYLQMNSLRAEDTAVYYCTVLRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 217 aa, Artificial Sequence; identical to SEQ ID NO: 10
  Description of Artificial Sequence Synthetic peptide

SEQ ID NO: 21 - protein, 118 aa, Artificial Sequence
  Description of Artificial Sequence Synthetic peptide
EVQLLESGGGLVQPGGSLRLSCAASGFTFSNNWMSWVRQAPGKGLEWVSAISGSGGSTYYADSVKGRFTISRDNSKNTLYLQMNSLRAEDTAVYYCNVQRRELGATNWGQGTLVTVSS

SEQ ID NO: 22 - protein, 5 aa, Artificial Sequence
  Description of Artificial Sequence Synthetic peptide
NNWMS

SEQ ID NO: 23 - protein, 17 aa, Artificial Sequence
  Description of Artificial Sequence Synthetic peptide
AISGSGGSTYYADSVKG

SEQ ID NO: 24 - protein, 9 aa, Artificial Sequence
  Description of Artificial Sequence Synthetic peptide
QRRELGATN

SEQ ID NO: 25 - protein, 111 aa, Artificial Sequence; identical to SEQ ID NO: 5
  Description of Artificial Sequence Synthetic peptide

SEQ ID NO: 26 - protein, 14 aa, Artificial Sequence
  Description of Artificial Sequence Synthetic peptide
TGSSSNIGAGYVVH

SEQ ID NO: 27 - protein, 7 aa, Artificial Sequence
  Description of Artificial Sequence Synthetic peptide
SNNQRPS

SEQ ID NO: 28 - protein, 11 aa, Artificial Sequence
  Description of Artificial Sequence Synthetic peptide
QSYDSSLSGVV

SEQ ID NO: 29 - protein, 447 aa, Artificial Sequence
  Description of Artificial Sequence Synthetic peptide
EVQLLESGGGLVQPGGSLRLSCAASGFTFSNNWMSWVRQAPGKGLEWVSAISGSGGSTYYADSVKGRFTISRDNSKNTLYLQMNSLRAEDTAVYYCNVQRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 217 aa, Artificial Sequence; identical to SEQ ID NO: 10
  Description of Artificial Sequence Synthetic peptide

SEQ ID NO: 31 - protein, 118 aa, Artificial Sequence
  Description of Artificial Sequence Synthetic peptide
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCTVQRRELGATNWGQGTLVTVSS

SEQ ID NO: 32 - protein, 5 aa, Artificial Sequence
  Description of Artificial Sequence Synthetic peptide
NYWMS

SEQ ID NO: 33 - protein, 17 aa, Artificial Sequence
  Description of Artificial Sequence Synthetic peptide
AISGSGGSTYYADSVKG

SEQ ID NO: 34 - protein, 9 aa, Artificial Sequence
  Description of Artificial Sequence Synthetic peptide
QRRELGATN

SEQ ID NO: 35 - protein, 111 aa, Artificial Sequence; identical to SEQ ID NO: 5
  Description of Artificial Sequence Synthetic peptide

SEQ ID NO: 36 - protein, 14 aa, Artificial Sequence
  Description of Artificial Sequence Synthetic peptide
TGSSSNIGAGYVVH

SEQ ID NO: 37 - protein, 7 aa, Artificial Sequence
  Description of Artificial Sequence Synthetic peptide
SNNQRPS

SEQ ID NO: 38 - protein, 11 aa, Artificial Sequence
  Description of Artificial Sequence Synthetic peptide
QSYDSSLSGVV

SEQ ID NO: 39 - protein, 447 aa, Artificial Sequence
  Description of Artificial Sequence Synthetic peptide
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCTVQRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 217 aa, Artificial Sequence; identical to SEQ ID NO: 10
  Description of Artificial Sequence Synthetic peptide

SEQ ID NO: 41 - protein, 118 aa, Artificial Sequence
  Description of Artificial Sequence Synthetic peptide
EVQLLESGGGLVQPGGSLRLSCAASGFTFSNYWMSWVRQAPGKGLEWVSAISGSGGTTYYADSVKGRFTISRDNSKNTLYLQMNSLRAEDTAVYYCTTQRRELGATNWGQGTLVTVSS

SEQ ID NO: 42 - protein, 5 aa, Artificial Sequence
  Description of Artificial Sequence Synthetic peptide
NYWMS

SEQ ID NO: 43 - protein, 17 aa, Artificial Sequence
  Description of Artificial Sequence Synthetic peptide
AISGSGGTTYYADSVKG

SEQ ID NO: 44 - protein, 9 aa, Artificial Sequence
  Description of Artificial Sequence Synthetic peptide
QRRELGATN

SEQ ID NO: 45 - protein, 111 aa, Artificial Sequence; identical to SEQ ID NO: 5
  Description of Artificial Sequence Synthetic peptide

SEQ ID NO: 46 - protein, 14 aa, Artificial Sequence
  Description of Artificial Sequence Synthetic peptide
TGSSSNIGAGYVVH

SEQ ID NO: 47 - protein, 7 aa, Artificial Sequence
  Description of Artificial Sequence Synthetic peptide
SNNQRPS

SEQ ID NO: 48 - protein, 11 aa, Artificial Sequence
  Description of Artificial Sequence Synthetic peptide
QSYDSSLSGVV

SEQ ID NO: 49 - protein, 447 aa, Artificial Sequence
  Description of Artificial Sequence Synthetic peptide
EVQLLESGGGLVQPGGSLRLSCAASGFTFSNYWMSWVRQAPGKGLEWVSAISGSGGTTYYADSVKGRFTISRDNSKNTLYLQMNSLRAEDTAVYYCTTQRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 217 aa, Artificial Sequence; identical to SEQ ID NO: 10
  Description of Artificial Sequence Synthetic peptide

SEQ ID NO: 51 - protein, 118 aa, Artificial Sequence
  Description of Artificial Sequence Synthetic peptide
EVQLLESGGGLVQPGGSLRLSCAASGFTFSNNWMSWVRQAPGKGLEWVSAISGSGGSTYYADSVKGRFTISRDNSKNTLYLQMNSLRAEDTAVYYCTVQRRELGATNWGQGTLVTVSS

SEQ ID NO: 52 - protein, 5 aa, Artificial Sequence
  Description of Artificial Sequence Synthetic peptide
NNWMS

SEQ ID NO: 53 - protein, 17 aa, Artificial Sequence
  Description of Artificial Sequence Synthetic peptide
AISGSGGSTYYADSVKG

SEQ ID NO: 54 - protein, 9 aa, Artificial Sequence
  Description of Artificial Sequence Synthetic peptide
QRRELGATN

SEQ ID NO: 55 - protein, 111 aa, Artificial Sequence; identical to SEQ ID NO: 5
  Description of Artificial Sequence Synthetic peptide

SEQ ID NO: 56 - protein, 14 aa, Artificial Sequence
  Description of Artificial Sequence Synthetic peptide
TGSSSNIGAGYVVH

SEQ ID NO: 57 - protein, 7 aa, Artificial Sequence
  Description of Artificial Sequence Synthetic peptide
SNNQRPS

SEQ ID NO: 58 - protein, 11 aa, Artificial Sequence
  Description of Artificial Sequence Synthetic peptide
QSYDSSLSGVV

SEQ ID NO: 59 - protein, 447 aa, Artificial Sequence
  Description of Artificial Sequence Synthetic peptide
EVQLLESGGGLVQPGGSLRLSCAASGFTFSNNWMSWVRQAPGKGLEWVSAISGSGGSTYYADSVKGRFTISRDNSKNTLYLQMNSLRAEDTAVYYCTVQRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 217 aa, Artificial Sequence; identical to SEQ ID NO: 10
  Description of Artificial Sequence Synthetic peptide

SEQ ID NO: 61 - protein, 118 aa, Artificial Sequence
  Description of Artificial Sequence Synthetic peptide
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCTTLRRELGATNWGQGTLVTVSS

SEQ ID NO: 62 - protein, 5 aa, Artificial Sequence
  Description of Artificial Sequence Synthetic peptide
NYWMS

SEQ ID NO: 63 - protein, 17 aa, Artificial Sequence
  Description of Artificial Sequence Synthetic peptide
AISGSGGSTYYADSVKG

SEQ ID NO: 64 - protein, 9 aa, Artificial Sequence
  Description of Artificial Sequence Synthetic peptide
LRRELGATN

SEQ ID NO: 65 - protein, 111 aa, Artificial Sequence; identical to SEQ ID NO: 5
  Description of Artificial Sequence Synthetic peptide

SEQ ID NO: 66 - protein, 14 aa, Artificial Sequence
  Description of Artificial Sequence Synthetic peptide
TGSSSNIGAGYVVH

SEQ ID NO: 67 - protein, 7 aa, Artificial Sequence
  Description of Artificial Sequence Synthetic peptide
SNNQRPS

SEQ ID NO: 68 - protein, 11 aa, Artificial Sequence
  Description of Artificial Sequence Synthetic peptide
QSYDSSLSGVV

SEQ ID NO: 69 - protein, 447 aa, Artificial Sequence
  Description of Artificial Sequence Synthetic peptide
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCTTLRRELGATNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 217 aa, Artificial Sequence; identical to SEQ ID NO: 10
  Description of Artificial Sequence Synthetic peptide

SEQ ID NO: 71 - protein, 123 aa, Artificial Sequence
  Description of Artificial Sequence Synthetic peptide
EVQLLESGGGLVQPGGSLRLSCAASGFTFSRYVMVWVRQAPGKGLEWVSGISPSGGVTRYAASVKGRFTISRDNSKNTLYLQMNSLRAEDTAVYYCARIRKEMTTISYFFDYWGQGTLVTVSS

SEQ ID NO: 72 - protein, 5 aa, Artificial Sequence
  Description of Artificial Sequence Synthetic peptide
RYVMV

SEQ ID NO: 73 - protein, 17 aa, Artificial Sequence
  Description of Artificial Sequence Synthetic peptide
GISPSGGVTRYAASVKG

SEQ ID NO: 74 - protein, 14 aa, Artificial Sequence
  Description of Artificial Sequence Synthetic peptide
IRKEMTTISYFFDY

SEQ ID NO: 75 - protein, 107 aa, Artificial Sequence
  Description of Artificial Sequence Synthetic peptide
DIQMTQSPSSLSASVGDRVTITCRASQGVDAYVAWYQQKPGKVPKLLIYSTSTLASGVPSRFSGSGSGTDFTLTISSLQPEDVATYYCQSHNAAVVTFGQGTRLEIK

SEQ ID NO: 76 - protein, 11 aa, Artificial Sequence
  Description of Artificial Sequence Synthetic peptide
RASQGVDAYVA

SEQ ID NO: 77 - protein, 7 aa, Artificial Sequence
  Description of Artificial Sequence Synthetic peptide
STSTLAS

SEQ ID NO: 78 - protein, 9 aa, Artificial Sequence
  Description of Artificial Sequence Synthetic peptide
QSHNAAVVT

SEQ ID NO: 79 - protein, 452 aa, Artificial Sequence
  Description of Artificial Sequence Synthetic peptide
EVQLLESGGGLVQPGGSLRLSCAASGFTFSRYVMVWVRQAPGKGLEWVSGISPSGGVTRYAASVKGRFTISRDNSKNTLYLQMNSLRAEDTAVYYCARIRKEMTTISYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 214 aa, Artificial Sequence
  Description of Artificial Sequence Synthetic peptide
DIQMTQSPSSLSASVGDRVTITCRASQGVDAYVAWYQQKPGKVPKLLIYSTSTLASGVPSRFSGSGSGTDFTLTISSLQPEDVATYYCQSHNAAVVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF